SEQUENCE LISTING (197 sequences)

SEQ ID NO: 1 - protein, 16 aa, Artificial Sequence
  Description of Artificial Sequence Synthetic ELABELA polypeptide signature sequence
  MOD_RES (2)..(4): Any amino acid
  MOD_RES (7)..(9): Any amino acid
CXXXRCXXXHSRVPFP

SEQ ID NO: 2 - protein, 32 aa, Homo sapiens
QRPVNLTMRRKLRKHNCLQRRCMPLHSRVPFP

SEQ ID NO: 3 - protein, 32 aa, Peromyscus sp.
QRPVNFPKKRKVYRHNCFRRRCVPLHSRVPFP

SEQ ID NO: 4 - protein, 32 aa, Rattus sp.
EKSVNFPRRRKLYRHNCFRRRCISLHSRVPFP

SEQ ID NO: 5 - protein, 32 aa, Mus musculus
QKPVNFPRRRKLYRHNCFRRRCIPLHSRVPFP

SEQ ID NO: 6 - protein, 32 aa, Bos sp.
QRQANLAMRRKLHRHNCLQRRCMPLHSRVPFP

SEQ ID NO: 7 - protein, 32 aa, Sus sp.
QRPANLAVRRKLHRHNCLQRRCMPLHSRVPFP

SEQ ID NO: 8 - protein, 32 aa, Dasypus sp.
QRPANLAMRRKLHRHNCFQRRCMPLHSRVPFP

SEQ ID NO: 9 - protein, 32 aa, Trichosurus sp.
QRPGNLALRRKPHRHICPQRRCMPLHSRVPFP

SEQ ID NO: 10 - protein, 32 aa, Gallus gallus
QRPANLALRRKLHRHNCSHRRCMPLHSRVPFP

SEQ ID NO: 11 - protein, 32 aa, Gekko sp.
QRPANLSLRRKLHRQHCSHRRCMPLHSRVPFP

SEQ ID NO: 12 - protein, 32 aa, Anolis sp.
QRPANLASRRKLHRHHCSHRRCMPLHSRVPFP

SEQ ID NO: 13 - protein, 32 aa, Xenopus laevis
QKPANLAQRRRIHRHNCFLKRCIPLHSRVPFP

SEQ ID NO: 14 - protein, 32 aa, Ambystoma sp.
QRPVNAAHRRRLHRHNCSLRRCMPLHSRVPFP

SEQ ID NO: 15 - protein, 32 aa, Oryzias sp.
ARPDFLNLRRKYHRHHCLHRRCMPLHSRVPFP

SEQ ID NO: 16 - protein, 32 aa, Callorhinchus sp.
QKSGNSWRRKKMQRRNCWHRRCLPFHSRVPFP

SEQ ID NO: 17 - protein, 32 aa, Oncorhynchus sp.
VRPDILNIRRRYHRHHCPHRRCMPLHSRVPFP

SEQ ID NO: 18 - protein, 36 aa, Danio rerio
DKHGTKHDFLNLRRKYRRHNCPKKRCLPLHSRVPFP

SEQ ID NO: 19 - protein, 22 aa, Homo sapiens
MRFQQFLFAFFIFIMSLLLISG

SEQ ID NO: 20 - protein, 54 aa, Homo sapiens
MRFQQFLFAFFIFIMSLLLISGQRPVNLTMRRKLRKHNCLQRRCMPLHSRVPFP

SEQ ID NO: 21 - protein, 54 aa, Peromyscus sp.
MRFQHYFLVFFIFAMSLLFITEQRPVNFPKKRKVYRHNCFRRRCVPLHSRVPFP

SEQ ID NO: 22 - protein, 54 aa, Rattus sp.
MRFQPLFWVFFIFAMSLLFITEEKSVNFPRRRKLYRHNCFRRRCISLHSRVPFP

SEQ ID NO: 23 - protein, 54 aa, Mus musculus
MRFQPLFWVFFIFAMSLLFISEQKPVNFPRRRKLYRHNCFRRRCIPLHSRVPFP

SEQ ID NO: 24 - protein, 54 aa, Bos sp.
MRFHQFFLLFVIFMLSLLLIHGQRQANLAMRRKLHRHNCLQRRCMPLHSRVPFP

SEQ ID NO: 25 - protein, 54 aa, Sus sp.
MRFRQFFLVFFIFMMNLLLICGQRPANLAVRRKLHRHNCLQRRCMPLHSRVPFP

SEQ ID NO: 26 - protein, 54 aa, Dasypus sp.
MKFQQFFYVFFVFIMSLLLINGQRPANLAMRRKLHRHNCFQRRCMPLHSRVPFP

SEQ ID NO: 27 - protein, 54 aa, Trichosurus sp.
MRFQLLFFLFLFFTMGILLIDGQRPGNLALRRKPHRHICPQRRCMPLHSRVPFP

SEQ ID NO: 28 - protein, 54 aa, Gallus gallus
MRLRRLLCVVFLLLVSLLPAAAQRPANLALRRKLHRHNCSHRRCMPLHSRVPFP

SEQ ID NO: 29 - protein, 54 aa, Gekko sp.
MRLQLLLLTCFLILTGVLLGNGQRPANLSLRRKLHRQHCSHRRCMPLHSRVPFP

SEQ ID NO: 30 - protein, 54 aa, Anolis sp.
MRLQQLLLTWFLLLAGALLINGQRPANLASRRKLHRHHCSHRRCMPLHSRVPFP

SEQ ID NO: 31 - protein, 54 aa, Xenopus laevis
MDFQKLLYALFFILMSLLLINGQKPANLAQRRRIHRHNCFLKRCIPLHSRVPFP

SEQ ID NO: 32 - protein, 54 aa, Ambystoma sp.
MKWQKLLAILFWILMGALLVNGQRPVNAAHRRRLHRHNCSLRRCMPLHSRVPFP

SEQ ID NO: 33 - protein, 54 aa, Oryzias sp.
MRVWNLLYLLLLLAAALAPVFSARPDFLNLRRKYHRHHCLHRRCMPLHSRVPFP

SEQ ID NO: 34 - protein, 54 aa, Callorhinchus sp.
MRFQHLLHIILLLCTSLLLISGQKSGNSWRRKKMQRRNCWHRRCLPFHSRVPFP

SEQ ID NO: 35 - protein, 53 aa, Oncorhynchus sp.
MRIISLLYLLLLVTVLGSVSSVRPDILNIRRRYHRHHCPHRRCMPLHSRVPFP

SEQ ID NO: 36 - protein, 58 aa, Danio rerio
MRFFHPLYLLLLLLTVLVLISADKHGTKHDFLNLRRKYRRHNCPKKRCLPLHSRVPFP

SEQ ID NO: 37 - DNA, 165 bp, Homo sapiens
atgagatttcagcaattcctttttgcattttttatttttattatgagtcttctccttatcagcggacagagaccagttaatttgaccatgagaagaaaactgcgcaaacacaattgccttcagaggagatgtatgcctctccattcacgagtacccttcccctga

SEQ ID NO: 38 - DNA, 165 bp, Mus musculus
atgcgattccagccccttttttgggtattttttatttttgccatgagtctcctttttatcagtgaacagaaaccagttaactttcccaggagaagaaaactatacagacacaactgctttcgcaggagatgcattccacttcattctcgagtgcccttcccatga

SEQ ID NO: 39 - DNA, 165 bp, Gallus gallus
atgaggctccggcggctgctgtgcgtcgtgttcctgctcctggtcagcctgctacctgccgccgcgcagagaccggccaacctggccctgcgcaggaagctgcaccgacacaactgctcgcaccggcggtgcatgccgctccactcccgcgtgccctttccctga

SEQ ID NO: 40 - DNA, 165 bp, Xenopus laevis
atggattttcagaaattattgtatgccttatttttcattctgatgagtctactgctgattaatggccagaaaccagccaacctcgcacagcgccggaggatacacagacacaactgcttcctcaagaggtgcataccactacattcaagagttccatttccatga

SEQ ID NO: 41 - DNA, 177 bp, Danio rerio
atgagattcttccacccgctgtatctgctgctgctgctgctgacagtgctggtcctcatcagcgcagataaacatggtacaaaacacgattttctcaacttgaggcggaaatatcgcagacacaactgcccgaagaaacgctgtctacctcttcactccagagtacctttcccttga

SEQ ID NO: 42 - DNA, 2422 bp, Homo sapiens
atcattaaccttcctgcaaaacacagctggcagttctctgaggcttgtcactagaatgtgaagacagccacacagatattgcacagactatttacagatcgtttggcttacattgagagtcattgctctacttttgtgcggtaggaaaatgagatttcagcaattcctttttgcattttttatttttattatgagtcttctccttatcagcggacagagaccagttaatttgaccatgagaagaaaactgcgcaaacacaattgccttcagaggagatgtatgcctctccattcacgagtacccttcccctgagatctctctagctaactttactggatctatcagaagaagaagaggagtgaaggaaagacacccagccacacaaaagaacttcatgatgccaacagcgtgattgcttagaagttcctacacaaaaaaaggatcatttgaaagcacctggaatggtttattagcttcacaggattttattcttcttggcttctatttggagggaaaataacataaattcaaaaggattccaatctgaagcccaaattgtttgcctacataacaaaaatatctcatcttttcctgcacattattattcttttatgggttaaaaagaaaaataccttttagtgttttagaactctctcatggtaaaaagtgcaagaatttaaaatgttgctttcatattcctataattctccaaaagtattaaattcgtatatgtttgagtgattttctaaaaactgctcaacctggaatcaattgcattgaccatttggcttcgcacaatagggagaaaataattggttcattgattatatagagagaaagactaagaaaagctattaattgctaccaattttatgataagctttaaggtttatgaaagtatgtttttttatttaatgagtaatgtccatttgaagttgaaagaaaacatgaaatcctaattgtagttcattttatgttcaaatgaaaccattgtttttgtttttgttttgaaacagagtctcactctgttgcccaaggtggagagaagtggcacgcttttgtctcactgcaacctccacctcccgagttcaagtgattctcgtgcctcaacctcccaattataggctgggattacaggtgtgcaccactacacccagctaattcttgtattttttgtagagatgaggttttaccctgttgcccaggctggtcttgaactcaggctggaaccattcattttttaacctttctcatcatgtaattataggaacccaacgtttgatttcctttgaagttttgttatgtcctttattattttgtatggataatttctttaaaagtcttacttaaagtcgacatctaaaatacagttatgccaatgaagtcccactcagggtgatatctgtatctaaaagatgagtgctcatcatcctattaggctttgtcttggtggtgttcatcctgagatgctgagacatggaaataaaaaatcagaaggaatttagggatatgattactcaaaaaagaaactatcctgtctaaatttgaattgtgttgataactaggtgttccccagatgctaagatgttcttaatttgtatttattgaaggattgttagcttagtgccacaaaatttttcttactttatgttaattccagataagaaatttacaagtttatatctttttttttcttttttttaagatgagatctggctctatcacccaggctaaagtgcagtggcatgatctaggctaactccctggctcaagcgatccttccacctcagcctcccaagtacctgggactacaggcactcacggccacacctgactaatttttgtatttttttgtagagatggagtatcgccatgttgcccaggttggtctcaaactcaggctggtgagctcaagtgatccgcctccttggcctcccaaaatactgggattacaggcatgtgccaccatgctgggccacaagttcatatctggagtagaagttttactttgtaaatattataaagtagaagaaaccataaaccattttgctaaaatgaaaggttggggttaatataaatgtaattttaaatagaaaatctgacaacactgtcgagtttgtcttcctgtcaaagcttattaaaagtgtctttgcggatgaatggtactttccacaagtgcatttgagtagaagcataacctattctcagttatatttatgtttaaaacatgtactggtttgtatattttgtactgaaaaagaaaacactttatagtcaagatacatctcattcaatacaagtctaaactctttcaaatacaaattcgcatattcacagaaaaagttacaaatcagttttactattgtaaagtaatgaaatggttatacatttcttaattgttcaataaaacactcaatgatt

SEQ ID NO: 43 - DNA, 975 bp, Mus musculus
cggactctccttggagctttgcagagacttcccgcttaagttactgcgtgcctgaatggaaaaggcagctggcagccctctgagttctggccataggatgtggggtgagccggacagatactgcgatttacagacgatttctctacttgaagagctattgttcacttgcgtgtaagaaaaaagaaaaacaaaaggaaagaaaaagaagaaaagaaaaaagagaaagaaaagaaaagatgcgattccagccccttttttgggtattttttatttttgccatgagtctcctttttatcagtgaacagaaaccagttaactttcccaggagaagaaaactatacagacacaactgctttcgcaggagatgcattccacttcattctcgagtgcccttcccatgaggaaaacctctaatttccttggctctaccagaagaagggtgaaagcaaagatacccaatcaccggaaaaacaacctcaggatggcaacaggatggcagctcaggagttactacgcaacagaggctgtttcagagtaccgtggatggcttttcagactgctcttcctggattctctttgacaagaaaatgatagaaagggaaaacagacgaggttaaagcacatgcgtttgtctgaataacaatctctcctgctgttctgcacgttctttgcgtataatgtatgaattacacatagtggtgggtttcacaaaggcatttacataccagtacatcacgtcctatggccgtctttccacacatgtgcatcatatacaccgattctttacaggcatgcatgccatatacactgactacatttgcacacacataccttcccctttctctcccctcaccctcctgcaggtctcttttattcacctgggcagttttgtttctattttaatggtttgtacacatgaatgattttattgcacattattattcttacatggattaaaaagaaaactactttaat

SEQ ID NO: 44 - DNA, 482 bp, Gallus gallus
ggcgagtgccacggacgcttctgtacacacgcggaccgcagggatgaggctccggcggctgctgtgcgtcgtgttcctgctcctggtcagcctgctacctgccgccgcgcagagaccggccaacctggccctgcgcaggaagctgcaccgacacaactgctcgcaccggcggtgcatgccgctccactcccgcgtgccctttccctgagcgcccggcccagctcggcaagcaatttcgtaacgggcttttcagtgtcttaaaggaggaagctgcaacaactgcactgatagagaagctcattctaagtactgcttaccaacagttgacctggtggagccacagcaatcctgttttgagggagtccatctgaaatgaacactttcagtggtcctgtgtatcacattctgcatgacctggaacaaaggcccatgactcatatcctagaagcagggggaagggagaaacggggaaggtgattggg

SEQ ID NO: 45 - DNA, 700 bp, Xenopus laevis
gtttcggtccggattcccgggatccagacctgatctaatagttgcctatctctgaagagcaatatggattttcagaaattattgtatgccttatttttcattctgatgagtctactgctgattaatggccagaaaccagccaacctcgcacagcgccggaggatacacagacacaactgcttcctcaagaggtgcataccactacattcaagagttccatttccatgagaccactgaggaatctcatcatgatgtaacataggatctgactcaaatctacagaaataatatttattttgaacagtgtttaagttgttctttgacttataagtggatgtttcttaaatgagctgcccacaagaatgggcacagacaagctccaacccatgtaactggctgtgaaactggcacaggagctgccgaaagcaatgtccctccatgaaactgatccaggagacatctacagcaacctttgttccaccaaaacagacaagagagcagattctccatcacattccaaggaagtgaatgttcaaaggcttgcatttattatcaaactgaaatgaagcatatcataataaggctcaatgtcctgtacctcaaaagatttaaaatatggggataaaatgaagaaaaacagaagatggtttttggacaatctggtcatatttttaaatattgccccgtgtgcaaaaaaaga

SEQ ID NO: 46 - DNA, 849 bp, Danio rerio
catcaggtcatctgtctatctatccatccctcagaggacagagagagaagagagagtgaatatcgccatctcaaactttgaaaaagttggagagaccgagagctggctagaactgcgcgtcttctatataactcaacttatacgagatctgagaacacttgctgagagcgacagacacataagaggatttctacagtccgttacctgcacatccgacagaatttatcgtctgaggaaccgcggacatcctgtgaggagagtcgagtctgcgccgcggaccaaaccaccctgagcatcaccatgagattcttccacccgctgtatctgctgctgctgctgctgacagtgctggtcctcatcagcgcagataaacatggtacaaaacacgattttctcaacttgaggcggaaatatcgcagacacaactgcccgaagaaacgctgtctacctcttcactccagagtacctttcccttgaggttttatgatgctccgggcaagcattaagaaaaaccaaagaccagccttggattggaaatgagaaaagatttatgtcagatgtgccgaggactgttttattcgcacatgtattgtaatcaaagccatgtttgtcacttctgtagcagaagtgttttttgttttgttttgttttttaaatgaatgtaagtgaatgagccatggagatcctactgctgccaaacatgctgcaaactcatcactcaatcaggttgagttggagcagaatcattgtaaatagtgaggactgaatgaaatgtgtttatatgtaagttatgcacttcaaatgttttattattatcttgatttattaaaagtgtattgtcttttcaga

SEQ ID NO: 47 - DNA, 19 bp, Artificial Sequence
  Description of Artificial Sequence Synthetic Anti-ELABELA shRNA sequence A oligonucleotide
gacacccagccacacaaaa

SEQ ID NO: 48 - DNA, 19 bp, Artificial Sequence
  Description of Artificial Sequence Synthetic Anti-ELABELA shRNA sequence B oligonucleotide
cccagccacacaaaagaac

SEQ ID NO: 49 - DNA, 19 bp, Artificial Sequence
  Description of Artificial Sequence Synthetic Anti-ELABELA shRNA sequence C oligonucleotide
gtgattctcgtgcctcaac

SEQ ID NO: 50 - DNA, 19 bp, Artificial Sequence
  Description of Artificial Sequence Synthetic Anti-ELABELA shRNA sequence D oligonucleotide
ctcacggccacacctgact

SEQ ID NO: 51 - DNA, 19 bp, Artificial Sequence
  Description of Artificial Sequence Synthetic Anti-ELABELA shRNA sequence E oligonucleotide
ttgccttcagaggagatgt

SEQ ID NO: 52 - protein, 11 aa, Homo sapiens
CMPLHSRVPFP

SEQ ID NO: 53 - protein, 17 aa, Homo sapiens
QRPVNLTMRRKLRKHNC

SEQ ID NO: 54 - protein, 10 aa, Unknown
  Description of Unknown ELABELA peptide
MPLHSRVPFP

SEQ ID NO: 55 - protein, 16 aa, Unknown
  Description of Unknown ELABELA peptide
QRPVNLTMRRKLRKHN

SEQ ID NO: 56 - protein, 54 aa, Artificial Sequence
  Description of Artificial Sequence Synthetic consensus polypeptide
  MOD_RES (2)..(2): Arg, Lys or Asp
  MOD_RES (3)..(3): Phe, Leu, Trp or Val
  MOD_RES (4)..(4): Gln, His, Arg or Trp
  MOD_RES (5)..(5): Gln, His, Pro, Leu, Arg, Lys or Asn
  MOD_RES (6)..(6): Phe, Tyr or Leu
  MOD_RES (7)..(7): Leu or Phe
  MOD_RES (8)..(8): Phe, Leu, Trp, Tyr, Cys, Ala or His
  MOD_RES (9)..(9): Ala, Val, Leu, Thr or Ile
  MOD_RES (10)..(10): Phe, Val, Cys, Trp, Leu or Ile
  MOD_RES (11)..(11): Phe, Val or Leu
  MOD_RES (12)..(12): Ile, Val, Phe, Leu or Trp
  MOD_RES (13)..(13): Phe, Leu or Ile
  MOD_RES (14)..(14): Ile, Ala, Met, Thr, Leu or Cys
  MOD_RES (15)..(15): Met, Leu, Val, Thr or Ala
  MOD_RES (16)..(16): Ser, Asn, Gly or Ala
  MOD_RES (17)..(17): Leu, Ile, Val or Ala
  MOD_RES (18)..(18): Leu or Ala
  MOD_RES (19)..(19): Leu, Phe or Pro
  MOD_RES (20)..(20): Ile, Ala, Gly or Val
  MOD_RES (21)..(21): Ser, Thr, His, Cys, Asn, Asp, Ala or Phe
  MOD_RES (22)..(22): Gly, Glu, Ala or Ser
  MOD_RES (23)..(23): Gln, Glu or Ala
  MOD_RES (24)..(24): Arg or Lys
  MOD_RES (25)..(25): Pro, Ser or Gln
  MOD_RES (26)..(26): Val, Ala, Gly or Asp
  MOD_RES (27)..(27): Asn or Phe
  MOD_RES (28)..(28): Leu, Phe, Ala or Ser
  MOD_RES (29)..(29): Thr, Pro, Ala, Ser, Asn or Trp
  MOD_RES (30)..(30): Met, Lys, Arg, Val, Leu, Ser, Gln or His
  MOD_RES (31)..(31): Arg or Lys
  MOD_RES (32)..(32): Arg or Lys
  MOD_RES (33)..(33): Lys or Arg
  MOD_RES (34)..(34): Leu, Val, Pro, Ile, Tyr or Met
  MOD_RES (35)..(35): Arg, Tyr, His or Gln
  MOD_RES (36)..(36): Lys or Arg
  MOD_RES (37)..(37): His, Gln or Arg
  MOD_RES (38)..(38): Asn, Ile or His
  MOD_RES (40)..(40): Leu, Phe, Pro, Ser or Trp
  MOD_RES (41)..(41): Gln, Arg, His or Leu
  MOD_RES (42)..(42): Arg or Lys
  MOD_RES (45)..(45): Met, Val, Ile or Leu
  MOD_RES (46)..(46): Pro or Ser
  MOD_RES (47)..(47): Leu or Phe
MXXXXXXXXXXXXXXXXXXXXXXXXXXXXXXXXXXXXXCXXXRCXXXHSRVPFP

SEQ ID NO: 57 - protein, 4 aa, Danio rerio
DKHG

SEQ ID NO: 58 - protein, 7 aa, Artificial Sequence
  Description of Artificial Sequence Synthetic ELABELA polypeptide signature sequence
HSRVPFP

SEQ ID NO: 59 - protein, 12 aa, Artificial Sequence
  Description of Artificial Sequence Synthetic ELABELA polypeptide signature sequence
  MOD_RES (3)..(5): Any amino acid
RCXXXHSRVPFP

SEQ ID NO: 60 - protein, 16 aa, Homo sapiens
CLQRRCMPLHSRVPFP

SEQ ID NO: 61 - protein, 16 aa, Peromyscus sp.
CFRRRCVPLHSRVPFP

SEQ ID NO: 62 - protein, 16 aa, Rattus sp.
CFRRRCISLHSRVPFP

SEQ ID NO: 63 - protein, 16 aa, Mus musculus
CFRRRCIPLHSRVPFP

SEQ ID NO: 64 - protein, 16 aa, Bos sp.
CLQRRCMPLHSRVPFP

SEQ ID NO: 65 - protein, 16 aa, Sus sp.
CLQRRCMPLHSRVPFP

SEQ ID NO: 66 - protein, 16 aa, Dasypus sp.
CFQRRCMPLHSRVPFP

SEQ ID NO: 67 - protein, 16 aa, Trichosurus sp.
CPQRRCMPLHSRVPFP

SEQ ID NO: 68 - protein, 16 aa, Gallus gallus
CSHRRCMPLHSRVPFP

SEQ ID NO: 69 - protein, 16 aa, Gekko sp.
CSHRRCMPLHSRVPFP

SEQ ID NO: 70 - protein, 16 aa, Anolis sp.
CSHRRCMPLHSRVPFP

SEQ ID NO: 71 - protein, 16 aa, Xenopus laevis
CFLKRCIPLHSRVPFP

SEQ ID NO: 72 - protein, 16 aa, Ambystoma sp.
CSLRRCMPLHSRVPFP

SEQ ID NO: 73 - protein, 16 aa, Oryzias sp.
CLHRRCMPLHSRVPFP

SEQ ID NO: 74 - protein, 16 aa, Callorhinchus sp.
CWHRRCLPFHSRVPFP

SEQ ID NO: 75 - protein, 16 aa, Oncorhynchus sp.
CPHRRCMPLHSRVPFP

SEQ ID NO: 76 - protein, 16 aa, Danio rerio
CPKKRCLPLHSRVPFP

SEQ ID NO: 77 - protein, 23 aa, Unknown
  Description of Unknown ELABELA peptide
  MOD_RES (1)..(1): Arg or Lys
  MOD_RES (2)..(7): Any amino acid
  MOD_RES (9)..(11): Any amino acid
  MOD_RES (14)..(16): Any amino acid
XXXXXXXCXXXRCXXXHSRVPFP

SEQ ID NO: 78 - protein, 24 aa, Unknown
  Description of Unknown ELABELA peptide
  MOD_RES (1)..(1): Arg or Lys
  MOD_RES (2)..(8): Any amino acid
  MOD_RES (10)..(12): Any amino acid
  MOD_RES (15)..(17): Any amino acid
XXXXXXXXCXXXRCXXXHSRVPFP

SEQ ID NO: 79 - protein, 24 aa, Unknown
  Description of Unknown ELABELA peptide
  MOD_RES (1)..(1): Arg or Lys
  MOD_RES (2)..(2): Arg or Lys
  MOD_RES (3)..(8): Any amino acid
  MOD_RES (10)..(12): Any amino acid
  MOD_RES (15)..(17): Any amino acid
XXXXXXXXCXXXRCXXXHSRVPFP

SEQ ID NO: 80 - protein, 22 aa, Peromyscus sp.
MRFQHYFLVFFIFAMSLLFITE

SEQ ID NO: 81 - protein, 22 aa, Rattus sp.
MRFQPLFWVFFIFAMSLLFITE

SEQ ID NO: 82 - protein, 22 aa, Mus musculus
MRFQPLFWVFFIFAMSLLFISE

SEQ ID NO: 83 - protein, 22 aa, Bos sp.
MRFHQFFLLFVIFMLSLLLIHG

SEQ ID NO: 84 - protein, 22 aa, Sus sp.
MRFRQFFLVFFIFMMNLLLICG

SEQ ID NO: 85 - protein, 22 aa, Dasypus sp.
MKFQQFFYVFFVFIMSLLLING

SEQ ID NO: 86 - protein, 22 aa, Trichosurus sp.
MRFQLLFFLFLFFTMGILLIDG

SEQ ID NO: 87 - protein, 22 aa, Gallus gallus
MRLRRLLCVVFLLLVSLLPAAA

SEQ ID NO: 88 - protein, 22 aa, Gekko sp.
MRLQLLLLTCFLILTGVLLGNG

SEQ ID NO: 89 - protein, 22 aa, Anolis sp.
MRLQQLLLTWFLLLAGALLING

SEQ ID NO: 90 - protein, 22 aa, Xenopus laevis
MDFQKLLYALFFILMSLLLING

SEQ ID NO: 91 - protein, 22 aa, Ambystoma sp.
MKWQKLLAILFWILMGALLVNG

SEQ ID NO: 92 - protein, 22 aa, Oryzias sp.
MRVWNLLYLLLLLAAALAPVFS

SEQ ID NO: 93 - protein, 22 aa, Callorhinchus sp.
MRFQHLLHIILLLCTSLLLISG

SEQ ID NO: 94 - protein, 24 aa, Unknown
  Description of Unknown ELABELA peptide
  MOD_RES (1)..(1): Arg or Lys
  MOD_RES (2)..(2): Ala or Gly
  MOD_RES (3)..(8): Any amino acid
  MOD_RES (10)..(12): Any amino acid
  MOD_RES (15)..(17): Any amino acid
XXXXXXXXCXXXRCXXXHSRVPFP

SEQ ID NO: 95 - protein, 24 aa, Unknown
  Description of Unknown ELABELA peptide
  MOD_RES (1)..(1): Ala or Gly
  MOD_RES (2)..(2): Arg or Lys
  MOD_RES (3)..(8): Any amino acid
  MOD_RES (10)..(12): Any amino acid
  MOD_RES (15)..(17): Any amino acid
XXXXXXXXCXXXRCXXXHSRVPFP

SEQ ID NO: 96 - protein, 24 aa, Unknown
  Description of Unknown ELABELA peptide
  MOD_RES (1)..(1): Ala or Gly
  MOD_RES (2)..(2): Ala or Gly
  MOD_RES (3)..(8): Any amino acid
  MOD_RES (10)..(12): Any amino acid
  MOD_RES (15)..(17): Any amino acid
XXXXXXXXCXXXRCXXXHSRVPFP

SEQ ID NO: 97 - protein, 6 aa, Artificial Sequence
  Description of Artificial Sequence Synthetic 6xHis tag
HHHHHH

SEQ ID NO: 98 - DNA, 18 bp, Artificial Sequence
  Description of Artificial Sequence Synthetic primer
agcaaaacccggaggagt

SEQ ID NO: 99 - DNA, 20 bp, Artificial Sequence
  Description of Artificial Sequence Synthetic primer
cacgagtaccctttccctga

SEQ ID NO: 100 - DNA, 20 bp, Artificial Sequence
  Description of Artificial Sequence Synthetic primer
tccagcagatgcaagaactc

SEQ ID NO: 101 - DNA, 20 bp, Artificial Sequence
  Description of Artificial Sequence Synthetic primer
ggaagcccaagaacctgaat

SEQ ID NO: 102 - DNA, 21 bp, Artificial Sequence
  Description of Artificial Sequence Synthetic primer
aatacttcccccacaacacaa

SEQ ID NO: 103 - DNA, 20 bp, Artificial Sequence
  Description of Artificial Sequence Synthetic primer
cgccctactcgtacatctcg

SEQ ID NO: 104 - DNA, 18 bp, Artificial Sequence
  Description of Artificial Sequence Synthetic primer
gtggggaggtcgaggttc

SEQ ID NO: 105 - DNA, 20 bp, Artificial Sequence
  Description of Artificial Sequence Synthetic primer
gctgtgacaggtacccaacc

SEQ ID NO: 106 - DNA, 20 bp, Artificial Sequence
  Description of Artificial Sequence Synthetic primer
tgcgggctactgaaaagttc

SEQ ID NO: 107 - DNA, 19 bp, Artificial Sequence
  Description of Artificial Sequence Synthetic primer
agccacatcgctcagacac

SEQ ID NO: 108 - DNA, 20 bp, Artificial Sequence
  Description of Artificial Sequence Synthetic primer
atcttgaccctccctggaat

SEQ ID NO: 109 - DNA, 21 bp, Artificial Sequence
  Description of Artificial Sequence Synthetic primer
aatgggcggagttatgatacc

SEQ ID NO: 110 - DNA, 21 bp, Artificial Sequence
  Description of Artificial Sequence Synthetic primer
gatcttcactccccttgttca

SEQ ID NO: 111 - DNA, 19 bp, Artificial Sequence
  Description of Artificial Sequence Synthetic primer
gctgtgttcagccagtgct

SEQ ID NO: 112 - DNA, 20 bp, Artificial Sequence
  Description of Artificial Sequence Synthetic primer
cgtctgctactgcttcatcg

SEQ ID NO: 113 - DNA, 20 bp, Artificial Sequence
  Description of Artificial Sequence Synthetic primer
cctcttgcgctatggacttc

SEQ ID NO: 114 - DNA, 20 bp, Artificial Sequence
  Description of Artificial Sequence Synthetic primer
ttcttccacccgctgtatct

SEQ ID NO: 115 - DNA, 21 bp, Artificial Sequence
  Description of Artificial Sequence Synthetic primer
ccacatcggcctgtgtatatc

SEQ ID NO: 116 - DNA, 19 bp, Artificial Sequence
  Description of Artificial Sequence Synthetic primer
ggctgggtgtctttccttc

SEQ ID NO: 117 - DNA, 20 bp, Artificial Sequence
  Description of Artificial Sequence Synthetic primer
ttgctattcttcggccagtt

SEQ ID NO: 118 - DNA, 18 bp, Artificial Sequence
  Description of Artificial Sequence Synthetic primer
gctggagttgctggaagc

SEQ ID NO: 119 - DNA, 18 bp, Artificial Sequence
  Description of Artificial Sequence Synthetic primer
ctctcccgcaccagtcat

SEQ ID NO: 120 - DNA, 19 bp, Artificial Sequence
  Description of Artificial Sequence Synthetic primer
agcgtcagcatcttgttgg

SEQ ID NO: 121 - DNA, 21 bp, Artificial Sequence
  Description of Artificial Sequence Synthetic primer
tgttctggaggtccatggtag

SEQ ID NO: 122 - DNA, 21 bp, Artificial Sequence
  Description of Artificial Sequence Synthetic primer
catgcaggtgagttgtcagaa

SEQ ID NO: 123 - DNA, 20 bp, Artificial Sequence
  Description of Artificial Sequence Synthetic primer
tgtaggccctgtttctcctg

SEQ ID NO: 124 - DNA, 19 bp, Artificial Sequence
  Description of Artificial Sequence Synthetic primer
gcccaatacgaccaaatcc

SEQ ID NO: 125 - DNA, 20 bp, Artificial Sequence
  Description of Artificial Sequence Synthetic primer
atggggagactaggctgtga

SEQ ID NO: 126 - DNA, 21 bp, Artificial Sequence
  Description of Artificial Sequence Synthetic primer
catatcaggttcacttccggg

SEQ ID NO: 127 - DNA, 18 bp, Artificial Sequence
  Description of Artificial Sequence Synthetic primer
ggcagcgatttcctcatc

SEQ ID NO: 128 - DNA, 18 bp, Artificial Sequence
  Description of Artificial Sequence Synthetic primer
ttctgccgcaaaggagtc

SEQ ID NO: 129 - DNA, 21 bp, Artificial Sequence
  Description of Artificial Sequence Synthetic primer
gctttttctggtcttccttgc

SEQ ID NO: 130 - DNA, 20 bp, Artificial Sequence
  Description of Artificial Sequence Synthetic primer
gcctgcaatccagtaggtct

SEQ ID NO: 131 - DNA, 20 bp, Artificial Sequence
  Description of Artificial Sequence Synthetic primer
ccggagcatcataaaacctc

SEQ ID NO: 132 - DNA, 19 bp, Artificial Sequence
  Description of Artificial Sequence Synthetic oligonucleotide
gtgattctcgtgcctcaac

SEQ ID NO: 133 - DNA, 16 bp, Artificial Sequence
  Description of Artificial Sequence Synthetic oligonucleotide
tccacccgctgtatct

SEQ ID NO: 134 - DNA, 15 bp, Artificial Sequence
  Description of Artificial Sequence Synthetic oligonucleotide
gctgctgctgacagt

SEQ ID NO: 135 - DNA, 21 bp, Artificial Sequence
  Description of Artificial Sequence Synthetic oligonucleotide
aacacttgctgagagcgacag

SEQ ID NO: 136 - DNA, 22 bp, Artificial Sequence
  Description of Artificial Sequence Synthetic oligonucleotide
agatgtggtggtgttgagtagc

SEQ ID NO: 137 - DNA, 19 bp, Artificial Sequence
  Description of Artificial Sequence Synthetic primer
atggagccaacgccggaat

SEQ ID NO: 138 - DNA, 20 bp, Artificial Sequence
  Description of Artificial Sequence Synthetic primer
atgaatgccatggacaacat

SEQ ID NO: 139 - DNA, 20 bp, Artificial Sequence
  Description of Artificial Sequence Synthetic primer
acacacatcccagccttttc

SEQ ID NO: 140 - DNA, 20 bp, Artificial Sequence
  Description of Artificial Sequence Synthetic primer
ccatccctcagaggacagag

SEQ ID NO: 141 - DNA, 19 bp, Artificial Sequence
  Description of Artificial Sequence Synthetic primer
tcacactttggtggccagc

SEQ ID NO: 142 - DNA, 19 bp, Artificial Sequence
  Description of Artificial Sequence Synthetic primer
tcacaccttcgtagccagc

SEQ ID NO: 143 - DNA, 20 bp, Artificial Sequence
  Description of Artificial Sequence Synthetic primer
ccaatttcattcggcaatct

SEQ ID NO: 144 - DNA, 20 bp, Artificial Sequence
  Description of Artificial Sequence Synthetic primer
catgtttggcagcagtagga

SEQ ID NO: 145 - DNA, 43 bp, Artificial Sequence
  Description of Artificial Sequence Synthetic primer
ctttgtggtgaccctgcccctgttggccgtctacactgctctg

SEQ ID NO: 146 - DNA, 43 bp, Artificial Sequence
  Description of Artificial Sequence Synthetic primer
cagagcagtgtagacggccaacaggggcagggtcaccacaaag

SEQ ID NO: 147 - DNA, 23 bp, Artificial Sequence
  Description of Artificial Sequence Synthetic primer
atggaggaaggtggtgattttga

SEQ ID NO: 148 - DNA, 22 bp, Artificial Sequence
  Description of Artificial Sequence Synthetic primer
atggacccagaagaaacttcag

SEQ ID NO: 149 - DNA, 19 bp, Artificial Sequence
  Description of Artificial Sequence Synthetic primer
atgagattcttccacccgc

SEQ ID NO: 150 - DNA, 22 bp, Artificial Sequence
  Description of Artificial Sequence Synthetic primer
ggagacccttgtggttgactag

SEQ ID NO: 151 - DNA, 23 bp, Artificial Sequence
  Description of Artificial Sequence Synthetic primer
ttagagtgacacagacctcttcc

SEQ ID NO: 152 - DNA, 22 bp, Artificial Sequence
  Description of Artificial Sequence Synthetic primer
tcaagggaaaggtactctggag

SEQ ID NO: 153 - DNA, 19 bp, Artificial Sequence
  Description of Artificial Sequence Synthetic oligonucleotide
acacgtaccgggactatga

SEQ ID NO: 154 - DNA, 19 bp, Artificial Sequence
  Description of Artificial Sequence Synthetic oligonucleotide
ccatcatgctgacctgtta

SEQ ID NO: 155 - DNA, 19 bp, Artificial Sequence
  Description of Artificial Sequence Synthetic oligonucleotide
gacctctgcgcctaattat

SEQ ID NO: 156 - DNA, 57 bp, Danio rerio
atgagattcttccacccgctgtatctgctgctgctgctgctgacagtgctggtcctc

SEQ ID NO: 157 - protein, 51 aa, Artificial Sequence
  Description of Artificial Sequence Synthetic polypeptide
MRFFHPLYLLVLISADKHGTKHDFLNLRRKYRRHNCPKKRCLPLHSRVPFP

SEQ ID NO: 158 - protein, 29 aa, Artificial Sequence
  Description of Artificial Sequence Synthetic peptide
MRFFHPLYLCQCWSSSAQINMVQNTIFST

SEQ ID NO: 159 - protein, 7 aa, Artificial Sequence
  Description of Artificial Sequence Synthetic peptide
MRFFHPL

SEQ ID NO: 160 - protein, 33 aa, Homo sapiens
GQRPVNLTMRRKLRKHNCLQRRCMPLHSRVPFP

SEQ ID NO: 161 - protein, 36 aa, Unknown
  Description of Unknown apelin peptide
LVQPRGSRNGPGPWQGGRRKFRRQRPRLSHKGPMPF

SEQ ID NO: 162 - protein, 23 aa, Unknown
  Description of Unknown ELABELA peptide
  MOD_RES (1)..(7): Any amino acid
  MOD_RES (9)..(11): Any amino acid
  MOD_RES (14)..(16): Any amino acid
XXXXXXXCXXXRCXXXHSRVPFP

SEQ ID NO: 163 - protein, 24 aa, Unknown
  Description of Unknown ELABELA peptide
  MOD_RES (1)..(8): Any amino acid
  MOD_RES (10)..(12): Any amino acid
  MOD_RES (15)..(17): Any amino acid
XXXXXXXXCXXXRCXXXHSRVPFP

SEQ ID NO: 164 - protein, 24 aa, Homo sapiens
RRKLRKHNCLQRRCMPLHSRVPFP

SEQ ID NO: 165 - protein, 24 aa, Peromyscus sp.
KRKVYRHNCFRRRCVPLHSRVPFP

SEQ ID NO: 166 - protein, 24 aa, Rattus sp.
RRKLYRHNCFRRRCISLHSRVPFP

SEQ ID NO: 167 - protein, 24 aa, Mus musculus
RRKLYRHNCFRRRCIPLHSRVPFP

SEQ ID NO: 168 - protein, 24 aa, Bos sp.
RRKLHRHNCLQRRCMPLHSRVPFP

SEQ ID NO: 169 - protein, 24 aa, Sus sp.
RRKLHRHNCLQRRCMPLHSRVPFP

SEQ ID NO: 170 - protein, 24 aa, Dasypus sp.
RRKLHRHNCFQRRCMPLHSRVPFP

SEQ ID NO: 171 - protein, 24 aa, Trichosurus sp.
RRKPHRHICPQRRCMPLHSRVPFP

SEQ ID NO: 172 - protein, 24 aa, Gallus gallus
RRKLHRHNCSHRRCMPLHSRVPFP

SEQ ID NO: 173 - protein, 24 aa, Gekko sp.
RRKLHRQHCSHRRCMPLHSRVPFP

SEQ ID NO: 174 - protein, 24 aa, Anolis sp.
RRKLHRHHCSHRRCMPLHSRVPFP

SEQ ID NO: 175 - protein, 24 aa, Xenopus laevis
RRRIHRHNCFLKRCIPLHSRVPFP

SEQ ID NO: 176 - protein, 24 aa, Ambystoma sp.
RRRLHRHNCSLRRCMPLHSRVPFP

SEQ ID NO: 177 - protein, 24 aa, Oryzias sp.
RRKYHRHHCLHRRCMPLHSRVPFP

SEQ ID NO: 178 - protein, 24 aa, Callorhinchus sp.
RKKMQRRNCWHRRCLPFHSRVPFP

SEQ ID NO: 179 - protein, 24 aa, Oncorhynchus sp.
RRRYHRHHCPHRRCMPLHSRVPFP

SEQ ID NO: 180 - protein, 24 aa, Danio rerio
RRKYRRHNCPKKRCLPLHSRVPFP

SEQ ID NO: 181 - protein, 23 aa, Homo sapiens
RKLRKHNCLQRRCMPLHSRVPFP

SEQ ID NO: 182 - protein, 23 aa, Peromyscus sp.
RKVYRHNCFRRRCVPLHSRVPFP

SEQ ID NO: 183 - protein, 23 aa, Rattus sp.
RKLYRHNCFRRRCISLHSRVPFP

SEQ ID NO: 184 - protein, 23 aa, Mus musculus
RKLYRHNCFRRRCIPLHSRVPFP

SEQ ID NO: 185 - protein, 23 aa, Bos sp.
RKLHRHNCLQRRCMPLHSRVPFP

SEQ ID NO: 186 - protein, 23 aa, Sus sp.
RKLHRHNCLQRRCMPLHSRVPFP

SEQ ID NO: 187 - protein, 23 aa, Dasypus sp.
RKLHRHNCFQRRCMPLHSRVPFP

SEQ ID NO: 188 - protein, 23 aa, Trichosurus sp.
RKPHRHICPQRRCMPLHSRVPFP

SEQ ID NO: 189 - protein, 23 aa, Gallus gallus
RKLHRHNCSHRRCMPLHSRVPFP

SEQ ID NO: 190 - protein, 23 aa, Gekko sp.
RKLHRQHCSHRRCMPLHSRVPFP

SEQ ID NO: 191 - protein, 23 aa, Anolis sp.
RKLHRHHCSHRRCMPLHSRVPFP

SEQ ID NO: 192 - protein, 23 aa, Xenopus laevis
RRIHRHNCFLKRCIPLHSRVPFP

SEQ ID NO: 193 - protein, 23 aa, Ambystoma sp.
RRLHRHNCSLRRCMPLHSRVPFP

SEQ ID NO: 194 - protein, 23 aa, Oryzias sp.
RKYHRHHCLHRRCMPLHSRVPFP

SEQ ID NO: 195 - protein, 23 aa, Callorhinchus sp.
KKMQRRNCWHRRCLPFHSRVPFP

SEQ ID NO: 196 - protein, 23 aa, Oncorhynchus sp.
RRYHRHHCPHRRCMPLHSRVPFP

SEQ ID NO: 197 - protein, 23 aa, Danio rerio
RKYRRHNCPKKRCLPLHSRVPFP

(19) United States
(12) Patent Application Publication
(10) Pub. No.: US 2020/0179487 A1
(43) Pub. Date: Jun. 11, 2020

(54) METHODS OF DIAGNOSIS AND TREATMENT OF PRE-ECLAMPSIA

(71) Applicant: AGENCY FOR SCIENCE, TECHNOLOGY AND RESEARCH, Singapore (SG)

(72) Inventors: Bruno REVERSADE, Singapore (SG); Lena Wai Mun HO, Singapore (SG)

(73) Assignee: AGENCY FOR SCIENCE, TECHNOLOGY AND RESEARCH, Singapore (SG)

(21) Appl. No.: 16/318,089

(22) PCT Filed: Jun. 28, 2017
(86) PCT No.: PCT/SG2017/050321

§ 371 Date: Jan. 15, 2019

(30) Foreign Application Priority Data
Jul. 15, 2016 (SG) 10201605841S

(51) Int. Cl. A61K 38/22; G01N 33/68; A61P 15/00; A61P 9/12

(52) U.S. Cl. CPC A61K 38/22; G01N 33/689; A61K 48/00; A61P 9/12; A61P 15/00

(57) ABSTRACT

The invention relates to an ELABELA polypeptide or nucleic acid for use in the treatment, alleviation or prophylaxis of pre-eclampsia in an individual. In another aspect of the invention, there is provided a method of diagnosing or detecting the susceptibility of an individual to pre-eclampsia, wherein the method comprises detecting modulation, preferably down-regulation of expression, amount or activity of ELABELA.

### CROSS-REFERENCE TO RELATED APPLICATIONS

This application is a 35 U.S.C. § 371 National Phase Entry of the International Application No. PCT/SG2017/050321 filed Jun. 28, 2017, which designates the U.S., and which claims benefit under 35 U.S.C. § 119 of the Singapore Application No. 10201605841S filed Jul. 15, 2016, country code SG, the contents of each of which are incorporated herein by reference in their entirety.

### SEQUENCE LISTING

The instant application contains a Sequence Listing which has been submitted electronically in ASCII format and is hereby incorporated by reference in its entirety. Said ASCII copy, created on Feb. 7, 2020, is named 049595_094260USPX_SL.txt and is 67,842 bytes in size.

### FIELD

This invention relates to the fields of medicine, cell biology, molecular biology and genetics. This invention relates to the field of medicine.

### BACKGROUND

The placenta is a mammalian-specific organ and a critical source of factors responsible for remodeling the maternal cardiovascular system to accommodate the needs of the growing fetus.

Defects in placentation often result in intrauterine growth restriction (IUGR) for the fetus, and gestational complications such as pre-eclampsia (PE) for the mother.

PE affects 5-8% of all pregnancies and remains the leading cause of fetal and maternal morbidity/mortality. Current challenges in PE include early detection and the availability of effective drugs that do not adversely affect fetal development.

ELABELA encodes an endogenous ligand for the Apelin Receptor (APLNR or APJ). It is first detected in pre-implantation human blastocysts and controls the self-renewal of embryonic stem cells (1). In the adult, its expression is restricted to a few tissues, including two endocrine organs, the kidneys and placenta (1). In rodents, the onset of Ela expression coincides with zygotic transcription (FIG. 6A), peaks at the blastocyst stage, and is similarly restricted in the adult. In lower vertebrates, Ela is required for proper endoderm development, and Ela-deficient zebrafish have profound cardiac malformations resulting from impaired migration of cardiac progenitors (2, 3). Zebrafish lacking both Ela and Apelin (Apln), the alternate ligand for Aplnr, have defects in vasculogenesis owing to impaired migration of angioblasts to the midline (4).

At present, the molecular effects of ELA signaling downstream of APLNR are unknown, and its involvement in mammalian development and physiology has not been addressed.

Chng et al (2013) described a novel hormone ELABELA, and showed it to be a hormone essential for heart development. ELABELA was shown to signal via the apelin receptor (APNJ or APNLR). Ho et al (2015) showed that ELABELA Is an endogenous growth factor that sustains hESC self-renewal via the PI3K/AKT pathway. Murza et al (2016) described a bioactive fragment of ELABELA that modulates vascular and cardiac functions. Perjés et al (2016) described the characterization of APELA in the adult heart. Helker et al (2015) showed that Elabela guides angioblasts to the midline during vasculogenesis.

Reference is also made to International Patent Publication WO 2015/084264 (PCT/SG2014/000574).

WO 2015/084264 describes the structure and function of ELABELA. ELABELA is shown to comprise a number of activities, including maintaining self-renewal of a stem cell, maintaining pluripotency of a stem cell, maintaining growth of a stem cell, promoting growth of a stem cell, inhibiting apoptosis, binding to the cell surface of an embryonic stem cell, biasing differentiation of a stem cell toward an endodermal or mesodermal lineage, binding to apelin receptor (APLNR), binding to CXCR4 receptor, activating the P13K/AKT pathway, cardioprotection, restoration or maintenance of cardiac function during ischemia and/or reperfusion, reduction of oxidative stress, reduction of infarct size and inhibition of HIV infection.

In WO 2015/084264, ELABELA is disclosed for use in the treatment, prophylaxis or alleviation of cardiac dysfunction, hypertension, or a cardiovascular anomaly in blood pressure, cardiac contractility or fluid balance; a cardiovascular disease such as cardiac hypertrophy, coronary artery disease (CAD), atherosclerosis, post-infarct treatment, myocardial ischemia-reperfusion injury or atrial fibrillation, coronary heart disease, heart failure, pulmonary arterial hypertension (PAH); a condition associated with high blood pressure, such as hypertension, angina, congestive heart failure or erectile dysfunction; and a condition associated with HIV infection, such as AIDS in an individual.

Pre-eclampsia affects 2-8% of pregnancies worldwide. Pre-eclampsia may contribute to one of the most common causes of death due to pregnancy. Pre-eclampsia usually occurs after 32 weeks; however, if it occurs earlier it is associated with worse outcomes. Women who have had pre-eclampsia are at increased risk of heart disease and stroke later in life.

### SUMMARY

Hypertension and pre-eclampsia are however altogether very different diseases. Therefore, one would not generally extrapolate findings from general hypertension to pre-eclampsia, which is a condition unique to the presence of a placenta.

According to a 1^{st} aspect of the present invention, we provide an ELABELA polypeptide or nucleic acid for use in the diagnosis, detection of susceptibility to, treatment, alleviation or prophylaxis of pre-eclampsia in an individual.

An ELABELA polypeptide may be used in the diagnosis, detection of susceptibility to, treatment, alleviation or prophylaxis of pre-eclampsia in an individual.

The ELABELA polypeptide may comprise a sequence CXXXRCXXXHSRVPFP (SEQ ID NO: 1), in which X represents an amino acid residue, or a fragment, homologue, variant or derivative thereof.

The ELABELA polypeptide may comprise a sequence SEQ ID NO: 162 (XXXXXXXCXXXRCXXXHSRVPFP), in which X represents an amino acid residue, and in which position 1 of SEQ ID NO: 162 comprises a basic amino acid residue, preferably K or R.

The ELABELA polypeptide may comprise a sequence SEQ ID NO: 163 (XXXXXXXXCXXXRCXXXHSRVPFP), in which X represents an amino acid residue, and in which position 1 of SEQ ID NO: 163 comprises a basic amino acid residue. The amino acid residue may comprise K or R.

The ELABELA polypeptide may comprise a sequence SEQ ID NO: 163 (XXXXXXXXCXXXRCXXXHSRVPFP), in which X represents an amino acid residue, in which positions 1 and 2 of SEQ ID NO: 163 comprise a pair of basic amino acid residues. The pair of basic amino acid residues may comprise KK, KR, RK or RR.

The ELABELA polypeptide may comprise a sequence selected from the group consisting of: SEQ ID NO: 2 to SEQ ID NO: 18. The ELABELA polypeptide may comprise a human ELABELA sequence shown as SEQ ID NO: 2.

The ELABELA polypeptide may further comprise a signal sequence. The signal sequence may comprise a human ELABELA signal sequence shown in SEQ ID NO: 19.

The ELABELA polypeptide may comprise a sequence selected from the group consisting of: SEQ ID NO: 20 to SEQ ID NO: 36. The ELABELA polypeptide may comprise a human ELABELA sequence shown as SEQ ID NO: 20.

The ELABELA polypeptide may comprise an intramolecular covalent bond between cysteine residues at or about positions 1 and 6, with reference to the numbering in the sequence CXXXRCXXXHSRVPFP (SEQ ID NO: 1). One or both cysteine residues may comprise a reduced cysteine having a sulfhydryl group.

The ELABELA polypeptide may comprise a mutation of a basic residue at position 31. The ELABELA polypeptide may be such that a basic residue at position 31 is mutated to a neutral residue. The ELABELA polypeptide may be such that K or R at position 31 is mutated to A or G.

The ELABELA polypeptide may comprise a mutation of a basic residue at position 32. The ELABELA polypeptide may be such that a basic residue at position 32 is mutated to a neutral residue. The ELABELA polypeptide may be such that K or R at position 32 is mutated to A or G.

The ELABELA polypeptide may comprise a R31G, R31A, K31G or K31A substitution with reference to the position numbering of a human ELABELA sequence shown as SEQ ID NO: 20. The ELABELA polypeptide may comprise an R32G, R32A, K32G or K32A substitution with reference to the position numbering of a human ELABELA sequence shown as SEQ ID NO: 20.

An ELABELA nucleic acid may be used in the diagnosis, detection of susceptibility to, treatment, alleviation or prophylaxis of pre-eclampsia in an individual.

The ELABELA nucleic acid may comprises a sequence capable of encoding an ELABELA polypeptide as set out in the 1^{st} aspect of the invention.

The ELABELA nucleic acid may comprise a nucleic acid sequence shown in any of SEQ ID NO. 37 to SEQ ID NO: 46. The ELABELA nucleic acid may comprise a human ELABELA nucleic acid sequence SEQ ID NO: 37 or SEQ ID NO: 42.

There is provided, according to a 2^{nd} aspect of the present invention, a vector such as an expression vector comprising an ELABELA nucleic acid, a host cell such as a bacterial, fungal or yeast cell comprising such a vector or a nucleic acid, or a transgenic non-human animal comprising such a host cell, such a vector or such a nucleic acid, preferably a mammal such as a mouse for use in the diagnosis, detection of susceptibility to, treatment, alleviation or prophylaxis of pre-eclampsia in an individual.

We provide, according to a 3^{rd} aspect of the present invention, an shRNA or siRNA molecule capable of modulating any combination of the expression, amount or activity of an ELABELA polypeptide, preferably comprising a sequence selected from the group consisting of: SEQ ID NO: 47 to SEQ ID NO: 51 for use in the diagnosis, detection of susceptibility to, treatment, alleviation or prophylaxis of pre-eclampsia in an individual.

As a 4^{th} aspect of the present invention, there is provided an antibody or antigen-binding fragment thereof for use in the diagnosis, detection of susceptibility to, treatment, alleviation or prophylaxis of pre-eclampsia in an individual.

The antibody or antigen-binding fragment thereof may be capable of specifically binding to a polypeptide comprising the sequence CMPLHSRVPFP (SEQ ID NO: 52).

The antibody or antigen-binding fragment thereof may be capable of specifically binding to a polypeptide comprising the sequence QRPVNLTMRRKLRKHNC (SEQ ID NO: 53).

The antibody or antigen-binding fragment thereof may be capable of specifically binding to a polypeptide comprising the sequence QRPVNLTMRRKLRKHNCLQRRCMPLHSRVPFP (SEQ ID NO: 2).

The antibody or antigen-binding fragment thereof may be capable of specifically binding to an ELABELA polypeptide.

The antibody or antigen-binding fragment thereof may be capable of specifically binding to an ELABELA polypeptide encoded by an ELABELA nucleic acid.

The antibody or antigen-binding fragment thereof may be capable of specifically binding to an ELABELA polypeptide comprising the sequence of any of SEQ ID NOs: 1-36.

The antibody or antigen-binding fragment thereof may be capable of specifically binding to one or more of the above.

The antibody or antigen-binding fragment thereof may be further optionally comprise a label.

We provide, according to a 5^{th} aspect of the present invention, use of an ELABELA polypeptide or nucleic acid in the preparation of a medicament for the treatment or prevention of pre-eclampsia.

The ELABELA polypeptide may comprise a polypeptide as set out in in the 1^{st} aspect of the invention.

The ELABELA nucleic acid may comprise a nucleic acid as set out in the 2^{nd} aspect of the invention.

The present invention, in a 6^{th} aspect, provides a method of assaying a compound useful in the treatment or alleviation of pre-eclampsia.

The method may comprise contacting an ELABELA polypeptide with a candidate compound and performing an assay to determine if the candidate compound binds to the ELABELA polypeptide.

The method may comprise contacting an ELABELA polypeptide with a candidate compound and performing an assay to determine if the candidate compound modulates an activity of the ELABELA polypeptide.

The method may comprise contacting a cell expressing an ELABELA polypeptide with a candidate compound and performing an assay to determine if the candidate compound causes an elevated or reduced expression, amount or activity of the ELABELA polypeptide in or of the cell.

The method may comprise further comprising isolating or synthesising the compound of interest so identified.

In a 7^{th} aspect of the present invention, there is provided a method of treatment, alleviation or prophylaxis of pre-eclampsia in an individual. The method may comprise administering a therapeutically effective amount of ELABELA to the individual.

According to an 8^{th} aspect of the present invention, we provide a method of diagnosis or detection of susceptibility to pre-eclampsia in an individual. The method may comprise detecting modulation, preferably down-regulation, of expression, amount or activity of ELABELA in or of the individual.

The method may be such that down-regulation of ELABELA in or of the individual indicates pre-eclampsia or susceptibility or predisposition to pre-eclampsia in the individual.

### ELABELA

ELABELA is also known as ela, ELA, Apelin Receptor Early Endogenous Ligand, APELA, Toddler and Ende.

As the term is used in this document, “ELABELA” refers to ELABELA polypeptides as well as ELABELA nucleic acids. These are described in further detail below.

### Pre-Eclampsia

Pre-eclampsia or preeclampsia (PE) is a disorder of pregnancy characterized by high blood pressure and a large amount of protein in the urine. The disorder usually occurs in the third trimester of pregnancy and worsens over time.

In severe disease there may be red blood cell breakdown, a low blood platelet count, impaired liver function, kidney dysfunction, swelling, shortness of breath due to fluid in the lungs, or visual disturbances.

Pre-eclampsia increases the risk of poor outcomes for both the mother and the baby. If left untreated, it may result in seizures at which point it is known as eclampsia.

Causes and Risk Factors

There is no definitive known cause of pre-eclampsia. Risk factors include nulliparity (never given birth), diabetes mellitus, kidney disease, chronic hypertension, prior history of pre-eclampsia, family history of pre-eclampsia, advanced maternal age (>35 years), obesity, antiphospholipid antibody syndrome, multiple gestation, having donated a kidney, having sub-clinical hypothyroidism or thyroid antibodies and placental abnormalities such as placental ischemia.

While the exact cause of pre-eclampsia remains unclear, there is strong evidence that a major cause predisposing a susceptible woman to pre-eclampsia is an abnormally implanted placenta.

The abnormal implantation may stem from the maternal immune system's response to the placenta, specifically a lack of established immunological tolerance in pregnancy. This abnormally implanted placenta may result in poor uterine and placental perfusion, yielding a state of hypoxia and increased oxidative stress and the release of anti-angiogenic proteins along with inflammatory mediators into the maternal plasma. A major consequence of this sequence of events is generalized endothelial dysfunction.

Endothelial dysfunction is thought to result in hypertension and many of the other symptoms and complications associated with preclampsia.

Diagnostic Criteria

Pre-eclampsia is routinely screened for during prenatal care. Pre-eclampsia is diagnosed when a pregnant woman develops:
Blood pressure ≥140 mm Hg systolic or ≥90 mm Hg diastolic on two separate readings taken at least four to six hours apart after 20 weeks gestation in an individual with previously normal blood pressure.
In a woman with essential hypertension beginning before 20 weeks gestational age, the diagnostic criteria are: an increase in systolic blood pressure (SBP) of ≥30 mmHg or an increase in diastolic blood pressure (DBP) of ≥15 mmHg.
Proteinuria ≥0.3 grams (300 mg) or more of protein in a 24-hour urine sample or a SPOT urinary protein to creatinine ratio ≥0.3 or a urine dipstick reading of 1+ or greater (dipstick reading should only be used if other quantitative methods are not available).

The practice of this invention will employ, unless otherwise indicated, conventional techniques of chemistry, molecular biology, microbiology, recombinant DNA and immunology, which are within the capabilities of a person of ordinary skill in the art. Such techniques are explained in the literature. See, for example, J. Sambrook, E. F. Fritsch, and T. Maniatis, 1989, Molecular Cloning: A Laboratory Manual, Second Edition, Books 1-3, Cold Spring Harbor Laboratory Press; Ausubel, F. M. et al. (1995 and periodic supplements; Current Protocols in Molecular Biology, ch. 9, 13, and 16, John Wiley & Sons, New York, N.Y.); B. Roe, J. Crabtree, and A. Kahn, 1996, DNA Isolation and Sequencing: Essential Techniques, John Wiley & Sons; J. M. Polak and James O'D. McGee, 1990, In Situ Hybridization: Principles and Practice; Oxford University Press; M. J. Gait (Editor), 1984, Oligonucleotide Synthesis: A Practical Approach, Irl Press; D. M. J. Lilley and J. E. Dahlberg, 1992, Methods of Enzymology: DNA Structure Part A: Synthesis and Physical Analysis of DNA Methods in Enzymology, Academic Press; Using Antibodies: A Laboratory Manual: Portable Protocol NO. I by Edward Harlow, David Lane, Ed Harlow (1999, Cold Spring Harbor Laboratory Press, ISBN 0-87969-544-7); Antibodies: A Laboratory Manual by Ed Harlow (Editor), David Lane (Editor) (1988, Cold Spring Harbor Laboratory Press, ISBN 0-87969-314-2), 1855. Handbook of Drug Screening, edited by Ramakrishna Seethala, Prabhavathi B. Fernandes (2001, New York, N.Y., Marcel Dekker, ISBN 0-8247-0562-9); and Lab Ref A Handbook of Recipes, Reagents, and Other Reference Tools for Use at the Bench, Edited Jane Roskams and Linda Rodgers, 2002, Cold Spring Harbor Laboratory, ISBN 0-87969-630-3. Each of these general texts is herein incorporated by reference.

### BRIEF DESCRIPTION OF THE FIGURES

FIG. 1A shows that Exon 2 of Ela was flanked with loxp sites and removed with Zp3-cre recombinase to generate the [symbol] allele lacking the peptide-coding region.

FIG. 1B shows a schematic of wildtype and [symbol] cDNA transcript.

FIG. 1C shows a semi-quantitative PCR of Ela genomic locus and cDNA. Primer positions are indicated in A and B.

FIG. 1D shows that [symbol] from intercrosses and [symbol] (♀)× [symbol] (♂) crosses have reduced Mendelian representation at weaning. χ2=chisquare test; % P=penetrance; L=number of litters.

FIG. 1E depicts an [symbol] e10.5 embryo, indistinguishable from wildtype embryos.

FIG. 1F shows that 43% (n=17/39) of [symbol] embryos at e10.5 display cardiovascular defects.

FIG. 1G shows that 14% (n=3/22) of [symbol] embryos at e10.5 display cardiovascular defects.

FIG. 1H depicts an [symbol] e10.5 yolk sac, with normal vitelline vessels.

FIG. 1I shows [symbol] embryos at e10.5 with avascular yolk sacs with ruffled appearance.

FIG. 1J shows [symbol] embryos at e10.5 with avascular yolk sacs with ruffled appearance.

FIG. 1K shows CD31/Pecam staining of [symbol] , [symbol] (L), and [symbol] (M) e10.5 yolk sac vasculature.

FIG. 1N shows CD31 staining of [symbol] , [symbol] (O), and [symbol] (P) e10.5 head vasculature.

FIG. 1Q shows CD31 (green) and Smooth muscle actin (SMA) staining of [symbol] , [symbol] (R), and [symbol] (S) hearts.

FIG. 1T shows In situ hybridization (ISH) of Ela in e8 embryo, showing mRNA localization overlying the developing heart tube and hindgut region.

FIG. 1U shows RNAScope of Ela and Apj (V) in e8 embryo within deciduum.

FIG. 1W shows RNAScope of Ela and Apj (X) in e8 embryo in yolk sac layers. en=endoderm; me=mesoderm.

FIG. 2A shows that at e9.5, Ela is expressed in the chorionic plate (cp) of the chorioallantoic placenta.

FIG. 2B shows that at e9.5, Apj is expressed in fetal allantoic endothelial cells.

FIG. 2C shows that at e10.5, Ela expression in the placenta labyrinth (lb) is restricted to syncytiotrophoblasts.

FIG. 2D shows that at e10.5, Apj expression in the placenta labyrinth (lb) is restricted to endothelial cells adjacent to syncytiotrophoblasts.

FIG. 2E shows that Ela can be detected by immunohistochemistry (IHC) on e10.5 labyrinth sections where staining is most evident in the syncytiotrophoblasts lining blood sinuses and fetal endothelium (inset, arrowheads).

FIG. 2F shows that this staining, while diffuse, is abrogated in e10.5 [symbol] placentas.

FIG. 2G shows that ELISA detects Ela in maternal serum harvested at indicated gestational time points. (GD=gestational day; NP=non-pregnant)

FIG. 2H shows that ELISA of GDll maternal serum harvested from wildtype or [symbol] mothers mated with the wildtype or [symbol] fathers indicate contribution of Ela to the maternal circulation by both maternal and embryonic compartments.

FIG. 2I shows that H&E staining of [symbol] and [symbol] (J) e10.5 placentas showing poor invasion and vasculogenesis of [symbol] placentas. (al=allantois)

FIG. 2K shows CD31/Pecam staining of [symbol] and [symbol] (L) e10.5 showing an paucity of fetal endothelial cells in the labyrinth.

FIG. 2M shows quantification of e10.5 placentas showing significant reduction in labyrinth thickness in [symbol] as compared to [symbol] littermates. This reduction persists at e18.5 when comparing wildtype and [symbol] placentas. The latter are derived from two different litters which is possible at e18.5 when developmental variance is much smaller compared to at e10.5. Error bars=SEM; Statistics=unpaired 2-tail T-test.

FIG. 3A shows 24-hour urine protein/creatinine ratios from GD15 pregnant mothers of indicated genotype (♀) mated with fathers (♂) of indicated genotypes. Error bars=SEM; Statistics=unpaired 2-tail T-test.

FIG. 3B shows (B-I) Hematoxylin-stained sections (left) and transmission electron micrograph (TEM, increasing magnification from left to right 1Kx, 5Kx, 10Kx) of glomerular sections from GD18 wildtype mothers (mated to wildtype fathers) and [symbol] mothers (mated to [symbol] fathers) showing endotheliosis and endothelial deposits in the absence of Ela. (Scalebars: 2 μm).

FIG. 3J shows tail-cuff systolic blood pressure measurements of wildtype mothers (mated to wildtype fathers) and [symbol] mothers (mated to [symbol] fathers) at the indicated gestational age. Error bars=SEM; Statistics=unpaired 2-tail t-test; dotted line indicates day of parturition.

FIG. 3K shows BP reading from (C) calculated in the form of Delta BP (BP of indicated gestational day—baseline non-pregnant BP of the same mother). Error bars=SEM; statistics=unpaired 2-tail t-test.

FIG. 3L shows systolic BP measurements of wildtype mothers (mated to wildtype fathers) and [symbol] mothers (mated to [symbol] fathers) implanted at GD7 with infusion pumps containing either PBS or ELA peptide. Error bars=SEM; statistics=unpaired 2-tail t-test.

FIG. 3M shows 24-hour urine protein/creatinine ratios from subjects in (E) measured at GD15 and GD17. Error bars=SEM; statistics=unpaired 2-tail t-test.

FIG. 3N shows ELISA measurement of ELA levels in plasma of normal versus pre-eclamptic women in the third trimester of their pregnancy.

FIG. 4A shows that ELA and APELIN (B) activate APJ (APLNR) with the same potency as measured by beta-Arrestin recruitment.

FIG. 4C shows that ELA and APELIN have no appreciable synergy with respect to APJ activation.

FIG. 4D shows that ELA does not antagonize APELIN with respect to APJ activation.

FIG. 4E shows Delta BP measurements of pregnant mothers with the indicated genotypes and matings at GD16 and GD18. Data were acquired in conjunction with data presented in FIG. 3D—WT and Ela Δ/Δ readings are presented here again for comparison with the rest of the genotypes. Error bars=SEM; statistics=unpaired 2-tail t-test.

FIG. 4F shows 24-hour urine protein/creatinine ratios from subjects in (A) measured at GD15. Data were acquired in conjunction with data presented in FIG. 3A—WT and Ela Δ/Δ readings are presented here again for comparison with the rest of the genotypes. Error bars=SEM.

FIG. 4A illustrates a working model: Ela, produced by placental syncytium signals to Apj expressed on fetal endothelial cells to facilitate normal placentation, thereby preventing symptoms of pre-eclampsia during pregnancy. Apelin and Apj appear to have potentiating effects on preelampsic symptoms. Loss of Ela, in the presence of Apelin produced by fetal endothelial cells signaling through Apj, is responsible for placental defects predisposing the pregnant mother to pre-eclampsia.

FIG. 5A. Exon 3 of murine Ela was flanked with loxp sites and excised with cre recombinase to generate the [symbol] allele lacking the ELA mature peptide (MP)-coding region.

FIG. 5B. Schematic of cDNA from wt and [symbol] alleles. SP=signal peptide

FIG. 5C. Semi-quantitative PCR of Ela locus from gDNA and cDNA. Primer locations are indicated in FIG. 5B.

FIG. 5D. Distribution of genotypes at e10.5 and at weaning from intercrosses and [symbol] (mother)× [symbol] (father) crosses. % P=penetrance; L=number of litters. Data were tested using a Chi-square test with 1 degree of freedom for significant deviation from the expected distribution.

FIG. 5E to FIG. 5G. At e10.5, [symbol] embryos are indistinguishable from wt while 43% (n=17/39) of [symbol] embryos and 14% (n=3/22) of [symbol] embryos display cardiovascular defects along with intrauterine growth restriction. Scale bars: 1 mm.

FIG. 5H to FIG. 5J. At e10.5, [symbol] yolk sacs have normal vitelline vessels while affected [symbol] and [symbol] embryos have avascular yolk sacs with a ruffled appearance. Scale bars: 1 mm.

FIG. 5K to FIG. 5M. CD31 staining of [symbol] , [symbol] and [symbol] yolk sacs reveal poorly matured vasculature in mutant embryos. Scale bars: 50 μm.

FIG. 5N to FIG. 5P. CD31 staining of [symbol] , [symbol] and [symbol] head vasculature at e10.5. Scale bars: 300 μm.

FIG. 5Q to FIG. 5S. CD31 (green) and Smooth Muscle Actin (SMA) staining of [symbol] , [symbol] and [symbol] hearts. Scale bars: 300 μm.

FIG. 5T. In situ hybridization of Ela at e8, showing mRNA localization in the region overlying the developing heart tube (ht) and chordal neural hinge (cnh). Scale bars: 200 μm.

FIG. 5U to FIG. 5V. RNAScope of Ela and Apj in e8 embryo within its decidua showing expression in the primitive foregut (fg) and hindgut (hg) endoderm. Arrowheads indicate the start of Ela expression in the chorionic trophoblast. Scale bars: 100 μm.

FIG. 5W to FIG. 5X. RNAScope of Ela and Apj in e8 yolk sac layers adhering to underlying decidua. en=endoderm; me=mesoderm. Scale bars: 40 μm.

FIG. 6A. Semi-quantitative RT-PCR of early mouse preimplantation stage embryos showing no maternal expression of Elabela (Ela), which is first detected in 2-cell stage embryos. Oct4 and Nanog are control genes known to be maternal-zygotic and zygotic only, respectively.

FIG. 6B. Homologous recombination strategy used for removing exon 3 of mouse Ela.

FIG. 6C. Southern blots verifying correct insertion of the targeting vector shown in FIG. 6B. Clones 32 and 43 are correctly targeted and were used for blastocyst injection. Clone 45 was excluded due to undesired integration or rearrangements.

FIG. 6D. Phenotypic heterogeneity and classification of zygotic and maternal-zygotic [symbol] mice.

FIG. 6E to FIG. 6J. CD31 endothelial staining of the dorsal aorta and outflow tract

FIG. 6E, FIG. 6F and FIG. 6G. and intersomitic vessels

FIG. 6H, FIG. 6I and FIG. 6J. of [symbol] , [symbol] and [symbol] embryos. Scale bars: 300 μm.

FIG. 6K and FIG. 6L. Representative images of [symbol] and severely affected [symbol] embryos with cardiac edema and general growth retardation. Scale bars: 1 mm.

FIG. 6M and FIG. 6N. RNAscope of Ela and negative control in chorionic plate of e8.5 conceptus. Arrowhead shows the first signs of Ela expression in chorionic ectoderm. Scale bars: 50 μm.

FIG. 7A and FIG. 7B. At e9, Ela is expressed in the chorionic plate (cp) of the chorioallantoic placenta while its receptor Apj is expressed in fetal allantoic endothelial cells. d=decidua. Scale bars: 1 mm.

FIG. 7C and FIG. 7D. At e10.5, Ela expression in the placenta labyrinth (lb) is restricted to syncytiotrophoblasts (ST) while Apj expression is restricted to endothelial cells adjacent to ST. Scale bars: 100 μm.

FIG. 7C′ and FIG. 7D′. Higher magnification showing Ela expression in syncytiotrophoblasts (ST) surrounding maternal blood spaces (mbs), and Apj expression in endothelial cells (EC) lining fetal blood spaces (fbs). Scale bars: 200 μm.

FIG. 7E and FIG. 7F. ELA can be detected by immunohistochemistry using an ELA-specific antibody (α C) in wt e10.5 labyrinth in cells lining blood spaces (arrowheads) but not in [symbol] placentas. Scale bars: 20 μm.

FIG. 7G. ELISA detects circulating ELA in maternal serum harvested at indicated gestational days (GD) n=number of mice assayed at each gestational timepoint. NP=non-pregnant. Error bars indicate SEM of 3 independent experiments. Data were tested with one-way ANOVA (red asterisk) and with two sample Student's t-test (black asterisks).

FIG. 7H. ELISA of GD 10.5 maternal serum harvested from wt or [symbol] mothers mated with the wt or [symbol] fathers indicating a maternal and zygotic origin of circulating ELA during pregnancy. n=number of mice assayed at each gestational timepoint. Error bars indicate SEM of 3 independent experiments. Data were tested using one way ANOVA. In FIG. 7G and FIG. 7H, ELA detected in maternal zygotic knockout is attributed to assay background.

FIG. 7I and FIG. 7J. H&E staining of [symbol] and [symbol] e10.5 placentas showing poor invasion and angiogenesis of [symbol] placentas. Scale bars: 250 μm; al=allantois

FIG. 7K and FIG. 7L. CD31/Pecam-1 staining of e10.5 [symbol] and [symbol] showing a paucity of fetal endothelial cells in the labyrinth. Scale bars: 100 μm.

FIG. 7M and FIG. 7N. Alpp (Placenta Alkaline Phosphatase) staining of e10.5 [symbol] and [symbol] placentas showing lack of trophoblasts in the labyrinth. Scale bars: 50 μm. *p<0.05, **p<0.01 from indicated tests of significance.

FIG. 8A. RNAscope of Ela showing expression in endometrial stroma surrounding decidual (de) tissue of gestational day 8.5 uterus. Scale bar: 1 mm

FIG. 8B. RNAscope of Ela showing expression in collecting ducts of kidneys from a gestational day 10.5 mouse. Scale bar 50 μm.

FIG. 8C. qPCR of mRNA levels in kidneys of pregnant mice at indicated gestational timepoint. Each dot indicates left kidney from one mouse. Means of each gestational timepoint are not significantly different using one-way ANOVA.

FIG. 8D. Thickness of [symbol] and [symbol] placental labyrinths (from base of chorionic plate to the top of spongiotrophoblast layer) at e10.5.

FIG. 8E. Thickness of [symbol] and [symbol] placental labyrinths (from base of chorionic plate to the top of spongiotrophoblast layer) at e18.5. Each column represents one placenta, dots represent equally spaced lateral-to-medial measurements from each placenta. The means of the placenta from the two genotypes were tested with two-sample t-test with 6 degrees of freedom.

FIG. 8F and FIG. 8G. Immunofluorescence of apoptotic marker activated Caspase 3 in [symbol] and [symbol] placental labyrinths at e10.5. Scale bars: 200 μm.

FIG. 8H and FIG. 8I. Immunofluorescence of mitotic marker phosphorylated H3 in [symbol] and [symbol] placental labyrinths at e10.5. Scale bars: 200 μm.

FIG. 8J and FIG. 8K. Immunohistochemistry of syncytiotrophoblast marker Syncytin-1 in e10.5 [symbol] and [symbol] placental labyrinths. Scale bars: 50 μm.

Data are depicted as mean±s.e.m., **p<0.01, ***p<0.001 of two sample Student's T-test unless otherwise indicated.

FIG. 9A. Schematic of RNA-seq experiment of e9.5 wt versus [symbol] labyrinths.

FIG. 9B and FIG. 9C. GSEA analysis of [symbol] (Classes 1 and 3) showing an upregulation of hypoxic response and pro-angiogenic genes in [symbol] labyrinths, even in morphologically normal Class 1 placentas.

FIG. 9D. Gene ontology analysis of genes upregulated in [symbol] labyrinths. In red are pathways enriched in tip cells. P values are derived from a binomial distribution with Bonferroni correction.

FIG. 9E. GSEA detects an upregulation of endothelial tip cell genes in Class 1 [symbol] labyrinths.

FIG. 9F. qPCR validation of tip cell-enriched and angiogenic genes in [symbol] labyrinths (n=wt; n=6 [symbol] ). Error bars indicate SEM of 2 independent experiments.

FIG. 9G. Esm1 immunofluorescence on e9.5 placenta vibratome sections taken from medial planes containing the maternal central canal. Dotted line marks the position of the transitional zone. al=allantois, lb=labyrinth, d=decidua. Scale bars: 40 μm.

FIG. 9H. Number of Esm1⁺ cells per section (n=6 wt; n=7 [symbol] ; each section represents a distinct placenta)

FIG. 9I. 75^{th} percentile integrated density of Esm1⁺ cells in each placental sample quantified in FIG. 9H. Data presented as arbitrary units (A.U.)

FIG. 9J. Beta-galactosidase (LacZ transgene in [symbol] allele) staining of Ela^{+/+}; [symbol] and [symbol] ; [symbol] placentas indicate increased Apln expression in [symbol] labyrinths. Scale bars: 300 μm. *p<0.05, **p<0.01, ***p<0.001 of two sample Student's t-test.

FIG. 10A. Wholemount antibody staining of CD31 marking endothelial cells in embryos whose placentas were analysed by RNAseq in FIG. 9A. Scalebars: 250 μm.

FIG. 10B. Principal component analysis of RNA-seq data from wt, class 1 and 3 [symbol] placentas.

FIG. 10C and FIG. 10D. GSEA analysis of genes upregulated in [symbol] class 1 (FIG. 10C) and class 3 (FIG. 10D) placentas compared to wt showing an enrichment for genes in GSEA's hallmark hypoxia dataset.

FIG. 10E and FIG. 10F. Immunohistochemistry of hypoxia marker Hif1α in e10.5 [symbol] and [symbol] placental labyrinths and transitional zone. Scalebars: 150 μm

FIG. 10G and FIG. 10H. Immunofluorescence of prolyl-hydroxy Hif1α in [symbol] and [symbol] placental labyrinths at e10.5. Scale bars: 100 μm. de=decidua; TGC=trophoblasts giant cell; lb=labyrinth

FIG. 10I and FIG. 10J. β-galactosidase (β-gal) (LacZ transgene in [symbol] allele) staining of Ela^{+/+}; [symbol] and [symbol] ; [symbol] placentas, viewed from the fetal side, indicate increased Apln expression in [symbol] labyrinths.

FIG. 10K, FIG. 10L, FIG. 10M and FIG. 10N. Immunofluorescence of mitotic marker phosphorylated H3 in [symbol] (FIG. 10K) and class 1 (FIG. 10L, FIG. 10M) and class 3 (N) [symbol] yolk sac cross sections at e10.5. Scale bars: 200 μm.

FIG. 10O. Western blot of individual yolk sacs from [symbol] and [symbol] littermate control embryos at e10.5, showing increased mitotic marker phosphorylated H3.

FIG. 11A. Urine protein/creatinine ratios from GD 15 pregnant mothers (♀) of indicated genotype mated with fathers (♂) of indicated genotypes. Each dot represents an individual mouse. Error bars indicate SEM.

FIG. 11B. Repeated tail-cuff systolic blood pressure measurements of wt mothers (n=7, mated to wt fathers) and ElaΔ/Δ mothers (n=5, mated to ElaΔ/Δ fathers) at the indicated gestational age. Dotted line indicates day of parturition. Error bars indicate SEM. 2-way ANOVA analysis detected a significant interaction between time and genotype, F(7,49)=2.074; p=0.0413 i.e. ElaΔ/Δ females develop significantly higher systolic BP compared to the controls as pregnancy progressed. Asterisks indicate significance of two sample unpaired t-test between wt and ElaΔ/Δ on the indicated GD.

FIG. 11C. BP readings from FIG. 11B calculated in the form of Delta BP (BP of indicated GD-baseline non-pregnant BP of the same mother). Each dot represents BP of one mouse averaged over 20 readings; error bars indicate SEM. 2-way ANOVA test detected a statistically significant difference in mean Delta BP between wt and ElaΔ/Δ mice, F(1,16)=11.28, p=0.0100. Asterisks indicate significance of two sample unpaired t-test.

FIG. 11D. Weight of pups at e18.5 collected by caesarean section. Each dot represents one pup. Error bars indicate SEM.

FIG. 11E. Urine protein/creatinine ratios of wt mothers (mated to wt fathers) (black squares) and ElaΔ/Δ mothers (mated to ElaΔ/Δ fathers) (red circles) implanted at GD7 with infusion pumps containing either PBS (closed symbols) or synthetic ELA peptide (open symbols) measured at GD15. Each dot represents one mouse; error bars indicate SEM.

FIG. 11F. Systolic delta BP measurements of subjects in FIG. 11E measured at GD14, 16 and 18. Each dot represents one mouse; error bars indicate SEM. 2-way ANOVA test detected a statistically significant difference in mean Delta BP between ElaΔ/Δ+PBS and ElaΔ/Δ+ELA mice, F(1,16)=6.938, p=0.0300. Asterisks indicate significance of two sample unpaired t-test.

FIG. 11G. IHC of ELA with a C biotinylated ELA-specific antibody on human first trimester (8+3 weeks) placental FFPE sections. Scale bar=200 μm

FIG. 11H. Transwell invasion assay using Jar choriocarcinoma cells cultured in the presence of increasing concentrations of synthetic ELA peptide. Each dot represents the mean of 3 wells; error bars indicate SEM of 3 independent experiments.

FIG. 11I. Working model: mouse ELA, produced by ST cells signals to APJ expressed on fetal endothelial cells (ECs) to facilitate normal placental angiogenesis. ELA also enters the maternal circulation where it acts systemically to prevent symptoms of pre-eclampsia during pregnancy. Tb=trophoblast. Data are depicted as mean±s.e.m., *p<0.05, **p<0.01, ***p<0.001 of two-sample Student's t-test, unless otherwise stated.

FIG. 12A. GSEA analysis of genes upregulated in [symbol] class 1 placentas compared to wt showing an enrichment for genes in the interferon (IFN) response pathways.

FIG. 12B, FIG. 12C, FIG. 12D, FIG. 12E, FIG. 12F and FIG. 12G. Transmission electron micrographs of kidney cortex sections from wt and [symbol] gestational day 18.5 mice taken at 1000× (FIG. 12B, FIG. 12E); 5000× (FIG. 12C, FIG. 12F) and 15000× (FIG. 12D, FIG. 12C). Scale bars=2 μm.

FIG. 12H. Protein/Creatinine ratios of urine from non-pregnant wt and [symbol] females. Group means were tested with a Mann-Whitney test and found to be not significantly different.

FIG. 12I. qPCR analysis of e9.5 (top) and e18.5 (bottom) placentas for mRNAs of angiogenic genes. Each dot represents a single placenta. Data are depicted as mean±s.e.m., and means were tested using two sample Student's T-test, *p<0.05, **p<0.01, ***p<0.001.

FIG. 12J, FIG. 12K and FIG. 12L. ELISA measurements of maternal plasma harvested at gestational day (GD) 15 and 18 from wt and [symbol] pregnant mothers for soluble VEGFR1 (sFlt1) (FIG. 12J and FIG. 12K) and Vegfa (FIG. 12L). Group means were tested with a Mann Whitney test and found to be not significantly different.

FIG. 12M and FIG. 12N. RNAscope for ELA in chorionic villi of 3^{rd} trimester human placenta, with accompanying H&E stain to show expression of ELA in syncytiotrophobast (ST). Scale bars: 50 μm.

FIG. 13A. Delta systolic BP of wt and [symbol] pregnant mice at gestational (GD) 16 and 18. Means were not significantly different by unpaired two-sample Student's t-test.

FIG. 13B. Protein/Creatinine ratios of 12-hour urine samples from wt and [symbol] pregnant mice collected at GD15. In both FIG. 13A and FIG. 13B, datapoints of wt mice were also presented in FIG. 11A and FIG. 11C as these mice were part of the same cohort as [symbol] mice and were assayed together.

FIG. 13C. qPCR analysis of Esm1, Igfbp3 and Igfbp5 endothelial tip cell-enriched markers and angiogenic genes in wt and [symbol] e9.5 placentas (top panel) versus wt and [symbol] e9.5 placentas (bottom panel). Each dot represents one placenta.

FIG. 13D. Allantois from pre-fusion somite stage embryos (control and treatment matched for stage) were explanted and treated with 2.5 μM of ELA or APLN-36 or both for 12 hours, followed by digital droplet (dd) PCR analysis of Esm1 gene expression. Each dot represents one explant, and means were tested with a paired Student's t-test based on somitic stage of each explant.

FIG. 13E and FIG. 13E′. ddPCR analysis of 4-somite (4s) stage allantoic explants for genes involved in hypoxic response Foxoa3 and Hif3α.

FIG. 13F. ddPCR analysis of Apln mRNA levels in 4s and 5s stage allantoic explants treated with PBS (−) or 2.5 μM of ELA (+). In FIG. 13E and FIG. 13F, each dot represents one explant.

FIG. 13G. qPCR analysis of Apln mRNA levels from wt and [symbol] e9.5 placentas. In red are placentas from embryos with Class 3 phenotype, whereas black dots represent placentas from morphologically normal embryos.

FIG. 13H, FIG. 13H′, FIG. 13I and FIG. 13I′. RNAScope of Ela (FIG. 13H, FIG. 13H′) and Apln (FIG. 13I, FIG. 13I′) in e9 embryos within maternal decidua. Scale bars: 200 μm (top), 100 μm (bottom). In all panels, data are depicted as mean±s.e.m., *p<0.05, **p<0.01, ***p<0.001 of unpaired two sample Student's T-test, unless otherwise stated.

FIG. 14A. Periodic acid-Schiff (PAS) basal membrane staining of wt and [symbol] mice with respective treatments harvested at e18.5. Scale bars: 20 μm.

FIG. 14B and FIG. 14C. Immunofluorescence staining of Fibrinogen in kidneys of wt and [symbol] mice with respective treatments harvested at e18.5. Scale bars: 0.1 mm in FIG. 14B and 15 μm in FIG. 14C. Each panel represents a section from a distinct mouse.

### SEQUENCE LISTINGS

SEQ ID NO: 1 shows a sequence of an ELABELA polypeptide signature sequence. SEQ ID NO: 2 shows a sequence of a Homo ELABELA mature polypeptide. SEQ ID NO: 3 shows a sequence of a Peromyscus ELABELA mature polypeptide. SEQ ID NO: 4 shows a sequence of a Rattus ELABELA mature polypeptide.

SEQ ID NO: 5 shows a sequence of a Mus ELABELA mature polypeptide. SEQ ID NO: 6 shows a sequence of a Bos ELABELA mature polypeptide. SEQ ID NO: 7 shows a sequence of a Sus ELABELA mature polypeptide. SEQ ID NO: 8 shows a sequence of a Dasypus ELABELA mature polypeptide. SEQ ID NO: 9 shows a sequence of a Trichosurus ELABELA mature polypeptide.

SEQ ID NO: 10 shows a sequence of a Gallus ELABELA mature polypeptide. SEQ ID NO: 11 shows a sequence of a Gekko ELABELA mature polypeptide. SEQ ID NO: 12 shows a sequence of a Anolis ELABELA mature polypeptide. SEQ ID NO: 13 shows a sequence of a Xenopus ELABELA mature polypeptide. SEQ ID NO: 14 shows a sequence of a Ambystoma ELABELA mature polypeptide.

SEQ ID NO: 15 shows a sequence of a Oryzias ELABELA mature polypeptide. SEQ ID NO: 16 shows a sequence of a Callorhinchus ELABELA mature polypeptide. SEQ ID NO: 17 shows a sequence of a Oncorhynchus ELABELA mature polypeptide. SEQ ID NO: 18 shows a sequence of a Danio ELABELA mature polypeptide.

SEQ ID NO: 19 shows a sequence of a Human ELABELA signal sequence. SEQ ID NO: 20 shows a sequence of a Homo ELABELA polypeptide with signal sequence (bold). SEQ ID NO: 21 shows a sequence of a Peromyscus ELABELA polypeptide with signal sequence (bold). SEQ ID NO: 22 shows a sequence of a Rattus ELABELA polypeptide with signal sequence (bold).

SEQ ID NO: 23 shows a sequence of a Mus ELABELA polypeptide with signal sequence (bold). SEQ ID NO: 24 shows a sequence of a Bos ELABELA polypeptide with signal sequence (bold). SEQ ID NO: 25 shows a sequence of a Sus ELABELA polypeptide with signal sequence (bold). SEQ ID NO: 26 shows a sequence of a Dasypus ELABELA polypeptide with signal sequence (bold). SEQ ID NO: 27 shows a sequence of a Trichosurus ELABELA polypeptide with signal sequence (bold).

SEQ ID NO: 28 shows a sequence of a Gallus ELABELA polypeptide with signal sequence (bold). SEQ ID NO: 29 shows a sequence of a Gekko ELABELA polypeptide with signal sequence (bold). SEQ ID NO: 30 shows a sequence of a Anolis ELABELA polypeptide with signal sequence (bold). SEQ ID NO: 31 shows a sequence of a Xenopus ELABELA polypeptide with signal sequence (bold). SEQ ID NO: 32 shows a sequence of a Ambystoma ELABELA polypeptide with signal sequence (bold).

SEQ ID NO: 33 shows a sequence of a Oryzias ELABELA polypeptide with signal sequence (bold). SEQ ID NO: 34 shows a sequence of a Callorhinchus ELABELA polypeptide with signal sequence (bold). SEQ ID NO: 35 shows a sequence of a Oncorhynchus ELABELA polypeptide with signal sequence (bold). SEQ ID NO: 36 shows a sequence of a Danio ELABELA polypeptide with signal sequence (bold).

SEQ ID NO: 37 shows a Human (Homo sapiens) ELABELA cDNA sequence. SEQ ID NO: 38 shows a Mouse (Mus musculus) ELABELA cDNA sequence. SEQ ID NO: 39 shows a Chicken (Gallus gallus) ELABELA cDNA sequence. SEQ ID NO: 40 shows a Xenopus (Xenopus laevis) ELABELA cDNA sequence. SEQ ID NO: 41 shows a Zebrafish (Danio rerio) ELABELA cDNA sequence.

SEQ ID NO: 42 shows a Human (Homo sapiens) ELABELA genomic sequence. SEQ ID NO: 43 shows a Mouse (Mus musculus) ELABELA genomic sequence. SEQ ID NO: 44 shows a Chicken (Gallus gallus) ELABELA genomic sequence. SEQ ID NO: 45 shows a Xenopus (Xenopus laevis) ELABELA genomic sequence. SEQ ID NO: 46 shows a Zebrafish (Danio rerio) ELABELA genomic sequence.

SEQ ID NO: 47 shows a Anti-ELABELA shRNA sequence A. SEQ ID NO: 48 shows a Anti-ELABELA shRNA sequence B. SEQ ID NO: 49 shows a Anti-ELABELA shRNA sequence C. SEQ ID NO: 50 shows a Anti-ELABELA shRNA sequence D. SEQ ID NO: 51 shows a Anti-ELABELA shRNA sequence E.

### DETAILED DESCRIPTION

### Treatment and Diagnosis of Pre-Eclampsia

The Examples demonstrate a role for ELABELA in placental vasculogenesis.

Our data show that deletion of Ela causes midgestation death due to cardiovascular defects. We further demonstrate clearly that Ela is a placental hormone required for placental vasculogenesis, and that loss of Ela during pregnancy predisposes to pre-eclampsia

Finally, we show that deletion of Ela and Api have opposite effects on proteinuria and gestational blood pressure.

ELABELA, its variants, homologues, derivatives and fragments, as well as modulators such as agonists and antagonists, may therefore be used for the treatment, prophylaxis or alleviation of pre-eclampsia in an individual.

### ELABELA Polypeptides

We describe the use of an ELABELA polypeptide in the diagnosis, detection of susceptibility to, treatment, alleviation or prophylaxis of pre-eclampsia in an individual.

In the broadest sense, an ELABELA polypeptide is a polypeptide that includes an “ELABELA signature” sequence. An ELABELA polypeptide may further comprise one or more activities, such as a biological activity of a native ELABELA polypeptide, as described in this document. As is clear from a sequence alignment, such as set out in International Patent Publication WO 2015/084264, a number of ELABELA signatures are possible.

For example, an ELABELA signature may comprise the sequence HSRVPFP (SEQ ID NO: 57). Accordingly, as used in this document, the term “ELABELA polypeptide” may mean a polypeptide which comprises an HSRVPFP sequence (SEQ ID NO: 58). An ELABELA polypeptide comprising SEQ ID NO: 58 may comprise one or more biological activities of a native ELABELA polypeptide, such as maintaining self-renewal, pluripotency, or both of a stem cell, as described herein.

Alternatively, or in addition, an ELABELA signature may comprise the sequence RCXXXHSRVPFP (SEQ ID NO: 59). In this sense, therefore the term “ELABELA polypeptide” may mean a polypeptide which comprises an RCXXXHSRVPFP sequence (SEQ ID NO: 59), in which in which X represents any amino acid residue. An ELABELA polypeptide comprising SEQ ID NO: 59 may comprise one or more biological activities of a native ELABELA polypeptide, such as maintaining self-renewal, pluripotency, or both of a stem cell, as described herein.

In preferred embodiments, however, the ELABELA signature is intended to refer to a sequence CXXXRCXXXHSRVPFP (SEQ ID NO: 1), in which X signifies an amino acid residue. Accordingly, the term “ELABELA polypeptide” as used in this document is intended to refer to a sequence comprising a CXXXRCXXXHSRVPFP (SEQ ID NO: 1), in which X signifies an amino acid residue. An ELABELA polypeptide comprising SEQ ID NO: 1 may comprise one or more biological activities of a native ELABELA polypeptide, such as maintaining self-renewal, pluripotency, or both of a stem cell, as described herein.

For the purposes of this document, the term “ELABELA polypeptide” should also be taken to encompass any fragment, homologue, variant or derivative of such a polypeptide. Such ELABELA fragments, homologues, variants and derivatives are described in further detail elsewhere in this document. Such ELABELA fragments, homologues, variants and derivatives may comprise one or more biological activities of a native ELABELA polypeptide, such as maintaining self-renewal, pluripotency, or both of a stem cell, as described herein.

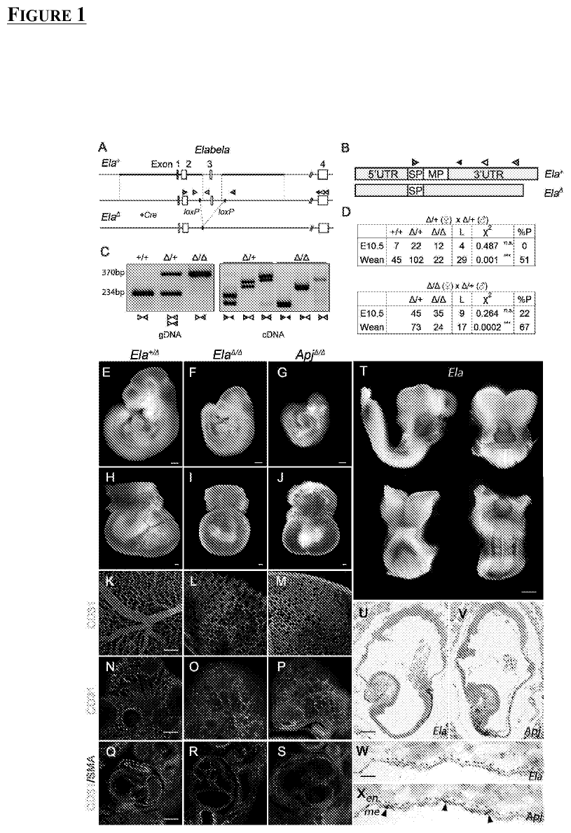

The ELABELA polypeptide encompassed by this document may therefore comprise a signature or conserved region from any of the vertebrate species in which ELABELA is expressed (see for example FIG. 1C of International Patent Publication WO 2015/084264). Such signatures and conserved regions are set out as SEQ ID NO: 2 to SEQ ID NO: 18 and SEQ ID NOs: 60 to 76.

The ELABELA polypeptide may comprise a signature or conserved region from any species, for example: a Homo sequence CLQRRCMPLHSRVPFP (SEQ ID NO: 60); a Peromyscus sequence CFRRRCVPLHSRVPFP (SEQ ID NO: 61); a Rattus sequence CFRRRCISLHSRVPFP (SEQ ID NO: 62); a Mus sequence CFRRRCIPLHSRVPFP (SEQ ID NO: 63); a Bos sequence CLQRRCMPLHSRVPFP (SEQ ID NO: 64); a Sus sequence CLQRRCMPLHSRVPFP (SEQ ID NO: 65); a Dasypus sequence CFQRRCMPLHSRVPFP (SEQ ID NO: 66); a Trichosurus sequence CPQRRCMPLHSRVPFP (SEQ ID NO: 67); a Gallus ELABELA polypeptide sequence CSHRRCMPLHSRVPFP (SEQ ID NO: 68); a Gekko sequence CSHRRCMPLHSRVPFP (SEQ ID NO: 69); a Anolis sequence CSHRRCMPLHSRVPFP (SEQ ID NO: 70); a Xenopus sequence CFLKRCIPLHSRVPFP (SEQ ID NO: 71); a Ambystoma sequence CSLRRCMPLHSRVPFP (SEQ ID NO: 72); a Oryzias sequence CLHRRCMPLHSRVPFP (SEQ ID NO: 73); a Callorhinchus sequence CWHRRCLPFHSRVPFP (SEQ ID NO: 74); a Oncorhynchus sequence CPHRRCMPLHSRVPFP (SEQ ID NO: 75); a Danio sequence CPKKRCLPLHSRVPFP (SEQ ID NO: 76).

The ELABELA polypeptide may therefore comprise a signature from human ELABELA, i.e., CXXXRCXXXHSRVPFP (SEQ ID NO: 1) may comprise CLQRRCMPLHSRVPFP (SEQ ID NO: 60). It may comprise a signature from mouse ELABELA, i.e., CXXXRCXXXHSRVPFP (SEQ ID NO: 1) may comprise CFRRRCIPLHSRVPFP (SEQ ID NO: 63).

An ELABELA polypeptide comprising CLQRRCMPLHSRVPFP (SEQ ID NO: 60) or CFRRRCIPLHSRVPFP (SEQ ID NO: 63) may comprise one or more biological activities of a native ELABELA polypeptide, such as maintaining self-renewal, pluripotency, or both of a stem cell, as described herein.

A number of other residues may also be present in the ELABELA polypeptide. For example, the ELABELA polypeptide may comprise one or more basic residues upstream of the ELABELA signature sequence.

In particular, the ELABELA polypeptide may comprise a basic residue at or about position −7 upstream of the ELABELA signature sequence CXXXRCXXXHSRVPFP (SEQ ID NO: 1). The basic residue may comprise a lysine residue, or an arginine residue.

Accordingly, the ELABELA polypeptide may comprise a sequence (R/K)XXXXXXCXXXRCXXXHSRVPFP (SEQ ID NO: 77) where X represents any amino acid.

An ELABELA polypeptide comprising (R/K)XXXXXXCXXXRCXXXHSRVPFP (SEQ ID NO: 77) may comprise one or more biological activities of a native ELABELA polypeptide, such as maintaining self-renewal, pluripotency, or both of a stem cell, as described herein.

The ELABELA polypeptide may, alternatively or in addition, comprise a basic residue at or about position −8 upstream of the ELABELA signature sequence CXXXRCXXXHSRVPFP (SEQ ID NO: 1). The basic residue may comprise a lysine residue, or an arginine residue.

Accordingly, the ELABELA polypeptide may comprise a sequence (R/K)XXXXXXXCXXXRCXXXHSRVPFP (SEQ ID NO: 78) where X represents any amino acid. An ELABELA polypeptide comprising (R/K)XXXXXXXCXXXRCXXXHSRVPFP (SEQ ID NO: 78) may comprise one or more biological activities of a native ELABELA polypeptide, such as maintaining self-renewal, pluripotency, or both of a stem cell, as described herein.

As noted above, the ELABELA polypeptide may comprise a pair of basic residues at or about positions −7 and −8 upstream of the ELABELA signature sequence CXXXRCXXXHSRVPFP (SEQ ID NO: 1), i.e., it may comprise a sequence (R/K)(R/K)XXXXXXCXXXRCXXXHSRVPFP (SEQ ID NO: 79).

It will be evident that where the ELABELA polypeptide comprises a signal sequence (see below), position −8 upstream of the ELABELA signature sequence CXXXRCXXXHSRVPFP (SEQ ID NO: 1) corresponds to position 31 of a human ELABELA sequence (SEQ ID NO: 20) and position −7 upstream of the ELABELA signature sequence CXXXRCXXXHSRVPFP (SEQ ID NO: 1) corresponds to position 32 of a human ELABELA sequence (SEQ ID NO: 20).

The ELABELA polypeptide may comprise a sequence selected from the group consisting of: SEQ ID NO: 2 to SEQ ID NO: 18. The ELABELA polypeptide may comprise a human ELABELA sequence shown as SEQ ID NO: 2. It may comprise a mouse ELABELA sequence shown as SEQ ID NO: 5.

In some embodiments, the ELABELA polypeptide comprises a signal peptide or signal sequence. The skilled reader will appreciate that the presence of a signal peptide will allow the ELABELA peptide to be exported and secreted from a cell. The skilled reader will also know how to engineer such signal sequences into the sequences of ELABELA polypeptides described in this document.

ELABELA polypeptides comprising signal sequences may referred to in this document for convenience as “full length” polypeptides. They may be produced by including any known signal sequences, including the ELABELA signal sequences disclosed in this document in an ELABELA polypeptide to be produced.

The ELABELA polypeptide may comprise an ELABELA signal sequence from any suitable species, for example, Homo MRFQQFLFAFFIFIMSLLLISG (SEQ ID NO: 19); Peromyscus MRFQHYFLVFFIFAMSLLFITE (SEQ ID NO: 80); Rattus MRFQPLFWVFFIFAMSLLFITE (SEQ ID NO: 81); Mus MRFQPLFWVFFIFAMSLLFISE (SEQ ID NO: 82); Bos MRFHQFFLLFVIFMLSLLLIHG (SEQ ID NO: 83); Sus MRFRQFFLVFFIFMMNLLLICG (SEQ ID NO: 84); Dasypus MKFQQFFYVFFVFIMSLLLING (SEQ ID NO: 85); Trichosurus MRFQLLFFLFLFFTMGILLIDG (SEQ ID NO: 86); Gallus MRLRRLLCVVFLLLVSLLPAAA (SEQ ID NO: 87); Gekko MRLQLLLLTCFLILTGVLLGNG (SEQ ID NO: 88); Anolis MRLQQLLLTWFLLLAGALLING (SEQ ID NO: 89); Xenopus MDFQKLLYALFFILMSLLLING (SEQ ID NO: 90); Ambystoma MKWQKLLAILFWILMGALLVNG (SEQ ID NO: 91); Oryzias MRVWNLLYLLLLLAAALAPVFS (SEQ ID NO: 92); or Callorhinchus MRFQHLLHIILLLCTSLLLISG (SEQ ID NO: 93).

It may for example comprise a human ELABELA signal sequence shown as SEQ ID NO: 19, i.e., MRFQQFLFAFFIFIMSLLLISG.

Examples of “full length” or “native” ELABELA polypeptides are disclosed herein, and include any of the sequences set out as SEQ ID NO: 20 to SEQ ID NO: 36. In some embodiments, the ELABELA polypeptide may comprise or consist of a human ELABELA polypeptide having or comprising a sequence shown as SEQ ID NO: 20. It may comprise or consist of a mouse ELABELA polypeptide, such as the sequence having SEQ ID NO: 23.

The “ELABELA polypeptide” may comprise one or more activities of a native ELABELA polypeptide, such as one or more biological activities of a native ELABELA polypeptide. Such ELABELA activities are described in detail elsewhere in this document, and include, for example, the ability to maintain self-renewal or pluripotency, or both, of a cell such as a stem cell.

As noted above, homologues variants and derivatives thereof of any, some or all of these polypeptides are also included in the term “ELABELA polypeptide”.

For example, an ELABELA polypeptide may comprise one or more reduced cysteines having a sulfhydryl group. The reduced cysteines may appear anywhere in the ELABELA amino acid sequence. For example, a cysteine at position 1 with reference to the numbering in the sequence CXXXRCXXXHSRVPFP (SEQ ID NO: 1) may comprise a reduced cysteine having a sulfhydryl group. A cysteine at position 6 may similarly comprise a reduced cysteine shaving a sulfhydryl group. The cysteine residues at both position 1 and position 6 may be so modified. The ELABELA polypeptide may comprise an intramolecular covalent bond between the cysteine residues at positions 1 and 6, with reference to the numbering in the sequence CXXXRCXXXHSRVPFP (SEQ ID NO: 1).

The position numberings above correspond respectively to position numbers 39 and 43 respectively in the human ELABELA polypeptide sequence MRFQQFLFAFFIFIMSLLLISGQRPVNLTMRRKLRKHNCLQRRCMPLHSRVPFP (SEQ ID NO: 20).

As another example, an ELABELA polypeptide may comprise one or more mutations of any of the sequences discussed in this document, such as those referred to as “ELABELA polypeptides”. Such mutated sequences are described in further detail elsewhere in this document.

Included are an ELABELA polypeptide which comprises a mutation of a basic residue at position 31 of its sequence, with reference to the position numbering of a human ELABELA sequence shown as SEQ ID NO: 20. The basic residue at position 31 may be mutated to a neutral residue. For example, an arginine or lysine residue at position 31 may be mutated to an alanine or glycine residue.

The ELABELA polypeptide may comprise a mutation of a basic residue at position 32, with reference to the position numbering of a human ELABELA sequence shown as SEQ ID NO: 20. For example, the basic residue at position 32 may be mutated to a neutral residue. Thus, an arginine or lysine residue at position 32 may be mutated to an alanine or glycine residue.

The ELABELA polypeptide may comprise a mutant in which both of the basic residues set out above are so mutated. Thus, the ELABELA polypeptide may comprise an R31G, R31A, K31G or K31A substitution. The substitutions may comprise R32G, R32A, K32G or K32A. The ELABELA polypeptide may therefore comprise any one of the sequences: (R/K)(A/G)XXXXXXCXXXRCXXXHSRVPFP (SEQ ID NO: 94), (A/G)(R/K)XXXXXXCXXXRCXXXHSRVPFP (SEQ ID NO: 95) or (A/G)(A/G)XXXXXXCXXXRCXXXHSRVPFP (SEQ ID NO: 96).

As noted above, the position numbering is with reference to the position numbering of a human ELABELA sequence shown as SEQ ID NO: 20.

It will be appreciated that, with reference to the position numbering of a human ELABELA sequence shown as SEQ ID NO: 20, positions 31 and 32 correspond to positions −8 and −7 respectively with respect to the position numbering of the ELABELA signature sequence CXXXRCXXXHSRVPFP (SEQ ID NO: 1).

As each of the ELABELA polypeptide sequences described in this document necessarily comprise an ELABELA signature sequence, the skilled person will be able to establish the position numbering of any particular residue within ELABELA polypeptide sequence in his possession. That is to say, a skilled person will, given the information available in this document, and in other resources he has in his possession, be able to establish, in any ELABELA polypeptide sequence, the position numbering of any particular residue with reference to the human ELABELA sequence shown as SEQ ID NO: 20 or with reference to the position numbering of the ELABELA signature sequence CXXXRCXXXHSRVPFP (SEQ ID NO: 1) comprised in the ELABELA polypeptide.

ELABELA polypeptides may be used for a variety of means, for example, administration to an individual suffering from, or suspected to be suffering from pre-eclampsia, for the treatment thereof.

They may also be used for production or screening of anti-ELABELA agents such as specific ELABELA binding agents, in particular, anti-ELABELA antibodies. These are described in further detail elsewhere in this document.

The expression of ELABELA polypeptides may be detected for diagnosis or detection of pre-eclampsia.

We specifically provide for ELABELA polypeptide fragments comprising a sequence CXXXRCXXXHSRVPFP (SEQ ID NO: 1), where X is an/any amino acid residue, and where the polypeptide fragment maintains self-renewal, pluripotency, or both of a stem cell.

The fragment may be such that it does not comprise a sequence of SEQ ID NOs: 60-76.

An intramolecular covalent bond may be present between the cysteine residues at positions 1 and 6 of SEQ ID NO: 1, or one or both cysteine residues at positions 1 and 6 of SEQ ID NO: 1 may comprise a reduced cysteine having a sulfhydryl group, or both.

The ELABELA polypeptide fragment may further comprise a label. The label may comprise a radioisotope. The radioisotope may comprise¹²⁵I.

The polypeptide fragment may be derivatized.

The ELABELA polypeptide fragment may further comprise a signal sequence. The signal sequence may comprise SEQ ID NO: 19.

The fragment may further comprise seven additional amino acids at the N-terminus of SEQ ID NO: 1. The ELABELA polypeptide fragment may have a sequence of SEQ ID NO: 162 (XXXXXXXCXXXRCXXXHSRVPFP). Position 1 of SEQ ID NO: 162 may comprise a basic amino acid residue. X at positions 2-6, 8-10, and 13-15 may comprise an/any amino acid residue. The polypeptide fragment may be capable of maintaining self-renewal, pluripotency, or both of a stem cell.

The fragment may be such that it does not comprise a sequence of SEQ ID NOs: 181-197.

The basic residue at the position 1 may be selected from K or R.

The fragment may further comprise eight additional amino acids at the N-terminus of SEQ ID NO: 1. The ELABELA polypeptide fragment may have a sequence of SEQ ID NO: 163 (XXXXXXXXCXXXRCXXXHSRVPFP). Position 1 of SEQ ID NO: 163 may comprise a basic amino acid residue. The X at positions 2-7, 9-11, and 14-17 may comprise an/any amino acid residue. The polypeptide fragment may be capable of maintaining self-renewal, pluripotency, or both of a stem cell.

The fragment may be such that it does not comprise a sequence of SEQ ID NOs: 164-180.

The basic residue at the position 1 may be selected from K or R.

The fragment may further comprise eight additional amino acids at the N-terminus of SEQ ID NO: 1. The ELABELA polypeptide fragment may comprise a sequence of SEQ ID NO: 163 (XXXXXXXXCXXXRCXXXHSRVPFP). Positions 1 and 2 of SEQ ID NO: 163 may comprise a pair of basic amino acid residues. The X at positions 3-7, 10-12, and 15-17 may comprise an/any amino acid residue. The polypeptide fragment may be capable of maintaining self-renewal, pluripotency, or both of a stem cell.

The fragment may be such that it does not comprise a sequence of SEQ ID NOs: 164-180.

The pair of basic residues at positions 1 and 2 may be selected from KK, KR, RK, and RR.

Polypeptide

A “polypeptide” refers to any peptide or protein comprising two or more amino acids joined to each other by peptide bonds or modified peptide bonds, i.e., peptide isosteres. “Polypeptide” refers to both short chains, commonly referred to as peptides, oligopeptides or oligomers, and to longer chains, generally referred to as proteins. Polypeptides may contain amino acids other than the 20 gene-encoded amino acids.

“Polypeptides” include amino acid sequences modified either by natural processes, such as post-translational processing, or by chemical modification techniques which are well known in the art. Such modifications are well described in basic texts and in more detailed monographs, as well as in a voluminous research literature. Modifications can occur anywhere in a polypeptide, including the peptide backbone, the amino acid side-chains and the amino or carboxyl termini. It will be appreciated that the same type of modification may be present in the same or varying degrees at several sites in a given polypeptide. Also, a given polypeptide may contain many types of modifications.

ELABELA polypeptides may be branched as a result of ubiquitination, and they may be cyclic, with or without branching. Cyclic, branched and branched cyclic polypeptides may result from posttranslation natural processes or may be made by synthetic methods. Modifications include acetylation, acylation, ADP-ribosylation, amidation, covalent attachment of flavin, covalent attachment of a heme moiety, covalent attachment of a nucleotide or nucleotide derivative, covalent attachment of a lipid or lipid derivative, covalent attachment of phosphotidylinositol, cross-inking, cyclization, disulfide bond formation, demethylation, formation of covalent cross-inks, formation of cystine, formation of pyroglutamate, formylation, gamma-carboxylation, glycosylation, GPI anchor formation, hydroxylation, iodination, methylation, myristoylation, oxidation, proteolytic processing, phosphorylation, prenylation, racemization, selenoylation, sulfation, transfer-RNA mediated addition of amino acids to proteins such as arginylation, and ubiquitination. See, for instance, Proteins—Structure and Molecular Properties, 2nd Ed., T. E. Creighton, W. H. Freeman and Company, New York, 1993 and Wold, F., Posttranslational Protein Modifications: Perspectives and Prospects, pgs. 1-12 in Posttranslational Covalent Modification of Proteins, B. C. Johnson, Ed., Academic Press, New York, 1983; Seifter et al., “Analysis for protein modifications and nonprotein cofactors”, Meth Enzymol (1990) 182:626-646 and Rattan et aL, “Protein Synthesis: Posttranslational Modifications and Aging”, Ann NY Acad Sci (1992) 663:48-62.

An ELABELA polypeptide may comprise a fragment having the sequence of SEQ ID NO: 53. Such a fragment may comprise a pyroglutamate at the N-terminus. As understood by one of ordinary skill in the art, when glutamic acid or glutamine are at the N-terminus of a polypeptide, such as an ELABELA polypeptide fragment of SEQ ID NO: 53, they can spontaneously cyclize to form pyroglutamate. In some embodiments, the fragment having the sequence of SEQ ID NO: 53 may further comprise a label.

The term “polypeptide” includes the various synthetic peptide variations known in the art, such as a retroinverso D peptides. The peptide may be an antigenic determinant and/or a T-cell epitope. The peptide may be immunogenic in vivo. The peptide may be capable of inducing neutralising antibodies in vivo.

As applied to ELABELA, the resultant amino acid sequence may have one or more activities, such as biological activities in common with an ELABELA polypeptide, for example a human ELABELA polypeptide. ELABELA polypeptide activities are described in detail elsewhere in this document. As an example, an ELABELA homologue may be capable of maintaining self-renewal or pluripotency, or both, of a cell such as a stem cell.

In particular, the term “homologue” is intended to cover identity with respect to structure and/or function providing the resultant amino acid sequence has ELABELA activity. With respect to sequence identity (i.e. similarity), there may be at least 70%, such as at least 75%, such as at least 85%, such as at least 90% sequence identity. There may be at least 95%, such as at least 98%, sequence identity. These terms also encompass polypeptides derived from amino acids which are allelic variations of the ELABELA nucleic acid sequence.

Other ELABELA Polypeptides

ELABELA variants, homologues, derivatives and fragments are also of use in the methods and compositions described here.

The terms “variant”, “homologue”, “derivative” or “fragment” in relation to ELABELA include any substitution of, variation of, modification of, replacement of, deletion of or addition of one (or more) amino acid from or to a sequence. Unless the context admits otherwise, references to “ELABELA” includes references to such variants, homologues, derivatives and fragments of ELABELA. Such ELABELA variants, homologues, derivatives and fragments may comprise one or more biological activities of a native ELABELA polypeptide, such as maintaining self-renewal, pluripotency, or both of a stem cell, as described herein.

As used herein a “deletion” is defined as a change in either nucleotide or amino acid sequence in which one or more nucleotides or amino acid residues, respectively, are absent.

As used herein an “insertion” or “addition” is that change in a nucleotide or amino acid sequence which has resulted in the addition of one or more nucleotides or amino acid residues, respectively, as compared to the naturally occurring substance.

As used herein “substitution” results from the replacement of one or more nucleotides or amino acids by different nucleotides or amino acids, respectively.

ELABELA polypeptides as described here may also have deletions, insertions or substitutions of amino acid residues which produce a silent change and result in a functionally equivalent amino acid sequence.

Deliberate amino acid substitutions may be made on the basis of similarity in polarity, charge, solubility, hydrophobicity, hydrophilicity, and/or the amphipathic nature of the residues. For example, negatively charged amino acids include aspartic acid and glutamic acid; positively charged amino acids include lysine and arginine; and amino acids with uncharged polar head groups having similar hydrophilicity values include leucine, isoleucine, valine, glycine, alanine, asparagine, glutamine, serine, threonine, phenylalanine, and tyrosine.

Conservative substitutions may be made, for example according to the table below. Amino acids in the same block in the second column and in the same line in the third column may be substituted for each other:

| | | |
|---|---|---|
| ALIPHATIC | Non-polar | GAP |
| | | ILV |
| | Polar-uncharged | CSTM |
| | | NQ |
| | Polar-charged | DE |
| | | KR |
| AROMATIC | | HFWY |

ELABELA polypeptides may further comprise heterologous amino acid sequences, typically at the N-terminus or C-terminus, such as the N-terminus.

Heterologous sequences may include sequences that affect intra or extracellular protein targeting (such as leader sequences). Heterologous sequences may also include sequences that increase the immunogenicity of the ELABELA polypeptide and/or which facilitate identification, extraction and/or purification of the polypeptides. Another heterologous sequence that may be used is a polyamino acid sequence such as polyhistidine which may be N-terminal. A polyhistidine sequence of at least 10 amino acids, such as at least 17 amino acids but fewer than 50 amino acids may be employed.

The ELABELA polypeptides may be in the form of the “mature” protein or may be a part of a larger protein such as a fusion protein. It is also possible to include an additional amino acid sequence which contains secretory or leader sequences, pro-sequences, sequences which aid in purification such as multiple histidine residues, or an additional sequence for stability during recombinant production.

The signal sequence (secretory sequence or leader sequence) may comprise the sequence MRFQQFLFAFFIFIMSLLLISG (SEQ ID NO: 19). An example of an ELABELA polypeptide which comprises such a signal sequence is the full length human ELABELA polypeptide sequence shown as SEQ ID NO: 20.

ELABELA polypeptides as described here may be made by recombinant means, using known techniques. However they may also be made by synthetic means using techniques well known to skilled persons such as using chemical methods, such as solid phase synthesis.

Such polypeptides may also be produced as fusion proteins, for example to aid in extraction and purification. Examples of fusion protein partners include glutathione-S-transferase (GST), 6×His (SEQ ID NO: 97), GAL4 (DNA binding and/or transcriptional activation domains) and β-galactosidase.

It may also be convenient to include a proteolytic cleavage site between the fusion protein partner and the protein sequence of interest to allow removal of fusion protein sequences, such as a thrombin cleavage site. The fusion protein may be one which does not hinder the function of the protein of interest sequence.

The ELABELA polypeptides may be in a substantially isolated form. This term is intended to refer to alteration by the hand of man from the natural state. If an “isolated” composition or substance occurs in nature, it has been changed or removed from its original environment, or both. For example, a polynucleotide, nucleic acid or a polypeptide naturally present in a living animal is not “isolated,” but the same polynucleotide, nucleic acid or polypeptide separated from the coexisting materials of its natural state is “isolated”, as the term is employed herein.

It will however be understood that the ELABELA protein may be mixed with carriers or diluents which will not interfere with the intended purpose of the protein and still be regarded as substantially isolated. An ELABELA polypeptide may also be in a substantially purified form, in which case it will generally comprise the protein in a preparation in which more than 90%, for example, 95%, 98% or 99% of the protein in the preparation is an ELABELA polypeptide.

By aligning ELABELA sequences from different species, it is possible to determine which regions of the amino acid sequence are conserved between different species (“homologous regions”), and which regions vary between the different species (“heterologous regions”).

An example of such an alignment is set out in FIG. 1C of International Patent Publication WO 2015/084264.

The ELABELA polypeptide may comprise a sequence which corresponds to at least part of a homologous region.

A homologous region shows a high degree of homology between at least two species. The two species may comprise for example human and another species, such as Peromyscus, Rattus, Mus, Bos, Sus, Dasypus, Trichosurus, Gallus, Gekko, Anolis, Xenopus, Ambystoma, Oryzias, Callorhinchus, Oncorhynchus or Danio.

The homologous region may for example show at least 70%, at least 80%, at least 90% or at least 95% identity at the amino acid level using the tests described above.

Examples of homologous regions are set out in this document and may comprise for example HSRVPFP (SEQ ID NO: 58), RCXXXHSRVPFP (SEQ ID NO: 59) or CXXXRCXXXHSRVPFP (SEQ ID NO: 1). Such homologous regions may comprise one or more biological activities of a native ELABELA polypeptide, such as maintaining self-renewal, pluripotency, or both of a stem cell, as described herein

Peptides which comprise a sequence which corresponds to a homologous region may be used in therapeutic strategies as explained in further detail elsewhere in this document. Alternatively, the ELABELA peptide may comprise a sequence which corresponds to at least part of a heterologous region. A heterologous region shows a low degree of homology between at least two species.

ELABELA Homologues

The ELABELA polypeptides disclosed for use include homologous sequences obtained from any source, for example related viral/bacterial proteins, cellular homologues and synthetic peptides, as well as variants or derivatives thereof.

Thus polypeptides also include those encoding homologues of ELABELA from other species including animals such as mammals (e.g. mice, rats or rabbits), especially primates, more especially humans. More specifically, homologues include human homologues.

In the context of this document, a homologous sequence is taken to include an amino acid sequence which is at least 15, at least 20, at least 25, at least 30, at least 40, at least 50, at least 60, at least 70, at least 80 or at least 90% identical, such as at least 95 or at least 98% identical at the amino acid level, for example over at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 or more amino acids with the sequence of a relevant ELABELA sequence.

In particular, homology should typically be considered with respect to those regions of the sequence known to be essential for protein function rather than non-essential neighbouring sequences. This is especially important when considering homologous sequences from distantly related organisms.

Examples of such regions in ELABELA are shown underlined in the sequence below:

(SEQ ID NO: 79)
(R/K)(R/K)XXXXXXCXXXRCXXXHSRVPFP

Although homology can also be considered in terms of similarity (i.e. amino acid residues having similar chemical properties/functions), in the context of the present document homology may be expressed in terms of sequence identity.

Homology comparisons can be conducted by eye, or more usually, with the aid of readily available sequence comparison programs. These publicly and commercially available computer programs can calculate % identity between two or more sequences.

% identity may be calculated over contiguous sequences, i.e. one sequence is aligned with the other sequence and each amino acid in one sequence directly compared with the corresponding amino acid in the other sequence, one residue at a time. This is called an “ungapped” alignment. Typically, such ungapped alignments are performed only over a relatively short number of residues (for example less than 50 contiguous amino acids).

Although this is a very simple and consistent method, it fails to take into consideration that, for example, in an otherwise identical pair of sequences, one insertion or deletion will cause the following amino acid residues to be put out of alignment, thus potentially resulting in a large reduction in % homology when a global alignment is performed. Consequently, most sequence comparison methods are designed to produce optimal alignments that take into consideration possible insertions and deletions without penalising unduly the overall homology score. This is achieved by inserting “gaps” in the sequence alignment to try to maximise local identity or similarity.

However, these more complex methods assign “gap penalties” to each gap that occurs in the alignment so that, for the same number of identical amino acids, a sequence alignment with as few gaps as possible—reflecting higher relatedness between the two compared sequences—will achieve a higher score than one with many gaps. “Affine gap costs” are typically used that charge a relatively high cost for the existence of a gap and a smaller penalty for each subsequent residue in the gap. This is the most commonly used gap scoring system. High gap penalties will of course produce optimised alignments with fewer gaps. Most alignment programs allow the gap penalties to be modified. However, the default values may be used when using such software for sequence comparisons. For example when using the GCG Wisconsin Bestfit package (see elsewhere in this document) the default gap penalty for amino acid sequences is −12 for a gap and −4 for each extension.

Calculation of maximum % homology therefore firstly requires the production of an optimal alignment, taking into consideration gap penalties. A suitable computer program for carrying out such an alignment is the GCG Wisconsin Bestfit package (University of Wisconsin, U.S.A.; Devereux et al., 1984, Nucleic Acids Research 12:387). Examples of other software than can perform sequence comparisons include, but are not limited to, the BLAST package (see Ausubel et al., 1999 ibid—Chapter 18), FASTA (Altschul et al., 1990, J. Mol. Biol., 403-410) and the GENEWORKS suite of comparison tools. Both BLAST and FASTA are available for offline and online searching (see Ausubel et al., 1999 ibid, pages 7-58 to 7-60). The GCG Bestfit program may be used.

Although the final % homology can be measured in terms of identity, the alignment process itself is typically not based on an all-or-nothing pair comparison. Instead, a scaled similarity score matrix is generally used that assigns scores to each pairwise comparison based on chemical similarity or evolutionary distance. An example of such a matrix commonly used is the BLOSUM62 matrix—the default matrix for the BLAST suite of programs. GCG Wisconsin programs generally use either the public default values or a custom symbol comparison table if supplied (see user manual for further details). The public default values for the GCG package may be used, or in the case of other software, the default matrix, such as BLOSUM62.

Once the software has produced an optimal alignment, it is possible to calculate % homology, such as % sequence identity. The software typically does this as part of the sequence comparison and generates a numerical result.

The terms “variant” or “derivative” in relation to amino acid sequences includes any substitution of, variation of, modification of, replacement of, deletion of or addition of one (or more) amino acids from or to the sequence providing the resultant amino acid sequence retains substantially the same activity as the unmodified sequence, such as having at least the same activity as the ELABELA polypeptides. Such ELABELA variants and derivatives may comprise one or more biological activities of a native ELABELA polypeptide, such as maintaining self-renewal, pluripotency, or both of a stem cell, as described herein.

Polypeptides having the ELABELA amino acid sequence disclosed here, or fragments or homologues thereof may be modified for use in the methods and compositions described here. Typically, modifications are made that maintain the biological activity of the sequence. Amino acid substitutions may be made, for example from 1, 2 or 3 to 10, 20 or 30 substitutions provided that the modified sequence retains the biological activity of the unmodified sequence. Alternatively, modifications may be made to deliberately inactivate one or more functional domains of the polypeptides described here. Amino acid substitutions may include the use of non-naturally occurring analogues, for example to increase blood plasma half-life of a therapeutically administered polypeptide.

ELABELA Fragments

Polypeptides for use in the methods and compositions described here also include fragments of the full length sequence of any of the ELABELA polypeptides identified above. Fragments may comprise at least one epitope. Methods of identifying epitopes are well known in the art. Fragments will typically comprise at least 5 amino acids, such as at least 6, 7, 8, 9, 10, 15, 20, 25, 30, 35, 40, 45, 50, 55 or more amino acids.

Included are fragments comprising or consisting of 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, or more residues from a relevant ELABELA amino acid sequence.

Such ELABELA fragments may comprise one or more biological activities of a native ELABELA polypeptide, such as maintaining self-renewal, pluripotency, or both of a stem cell, as described herein.

We further describe peptides comprising a portion of an ELABELA polypeptide as described here. Thus, fragments of ELABELA and its homologues, variants or derivatives are included. The peptides may be between 2 and 60 amino acids, such as between 4 and 50 amino acids in length. The peptide may be derived from an ELABELA polypeptide as disclosed here, for example by digestion with a suitable enzyme, such as trypsin. Alternatively the peptide, fragment, etc may be made by recombinant means, or synthesised synthetically via chemical means, such as solid phase synthesis.

We therefore describe a method of making an ELABELA polypeptide or a fragment, the method comprising expressing a nucleic acid encoding a sequence comprising CXXXRCXXXHSRVPFP (SEQ ID NO: 1) in a cell, in which X is an or any amino acid residue, and in which the polypeptide fragment maintains self-renewal, pluripotency or both of a stem cell.

The cell expressing the nucleic acid encoding a sequence comprising CXXXRCXXXHSRVPFP (SEQ ID NO: 1) may be a bacterial, fungal or yeast cell.

The sequence comprising CXXXRCXXXHSRVPFP (SEQ ID NO: 1) may be selected from the group consisting of SEQ ID NOs: 2 to 36.

We further describe a method of making an ELABELA polypeptide or a fragment, the method comprising using chemical synthesis to generate a synthetic polypeptide comprising CXXXRCXXXHSRVPFP (SEQ ID NO: 1) or a fragment having the sequence of SEQ ID NO: 53, in which X is an or any amino acid residue, and in which the polypeptide fragment maintains self-renewal, pluripotency, or both of a stem cell.

The ELABELA polypeptide or fragment, derivative, homologue or variant may comprise a label. The label may comprise a radioisotope. The radioisotope may comprise ¹²⁵I. the ELABELA polypeptide or fragment, derivative, homologue or variant may be derivatized.

Such ELABELA polypeptides, fragments, derivatives, homologues and variants may be used to generate probes to preferentially detect ELABELA expression, for example, through antibodies generated against such fragments. These antibodies would be expected to bind specifically to ELABELA, and are useful in the methods of diagnosis and treatment disclosed here.

ELABELA and its fragments, homologues, variants and derivatives, may be made by recombinant means. However they may also be made by synthetic means using techniques well known to skilled persons such as solid phase synthesis. The proteins may also be produced as fusion proteins, for example to aid in extraction and purification. Examples of fusion protein partners include glutathione-S-transferase (GST), 6×His (SEQ ID NO: 97), GAL4 (DNA binding and/or transcriptional activation domains) and β-galactosidase. It may also be convenient to include a proteolytic cleavage site between the fusion protein partner and the protein sequence of interest to allow removal of fusion protein sequences. The fusion protein may be one which will not hinder the function of the protein of interest sequence. Proteins may also be obtained by purification of cell extracts from animal cells.

The ELABELA polypeptides, variants, homologues, fragments and derivatives disclosed here may be in a substantially isolated form. It will be understood that such polypeptides may be mixed with carriers or diluents which will not interfere with the intended purpose of the protein and still be regarded as substantially isolated. An ELABELA variant, homologue, fragment or derivative may also be in a substantially purified form, in which case it will generally comprise the protein in a preparation in which more than 90%, e.g. 95%, 98% or 99% of the protein in the preparation is a protein.

The ELABELA polypeptides, variants, homologues, fragments and derivatives disclosed here may be labelled with a revealing label. The revealing label may be any suitable label which allows the polypeptide, etc to be detected. Suitable labels include radioisotopes, e.g. ¹²⁵I, enzymes, antibodies, polynucleotides and linkers such as biotin. Labelled polypeptides may be used in diagnostic procedures such as immunoassays to determine the amount of a polypeptide in a sample. Polypeptides or labelled polypeptides may also be used in serological or cell-mediated immune assays for the detection of immune reactivity to said polypeptides in animals and humans using standard protocols.

ELABELA polypeptides, variants, homologues, fragments and derivatives disclosed here, optionally labelled, may also be fixed to a solid phase, for example the surface of an immunoassay well or dipstick. Such labelled and/or immobilised polypeptides may be packaged into kits in a suitable container along with suitable reagents, controls, instructions and the like. Such polypeptides and kits may be used in methods of detection of antibodies to the polypeptides or their allelic or species variants by immunoassay.

Immunoassay methods are well known in the art and will generally comprise: (a) providing a polypeptide comprising an epitope bindable by an antibody against said protein; (b) incubating a biological sample with said polypeptide under conditions which allow for the formation of an antibody-antigen complex; and (c) determining whether antibody-antigen complex comprising said polypeptide is formed.

The ELABELA polypeptides, variants, homologues, fragments and derivatives disclosed here may be used in in vitro or in vivo cell culture systems to study the role of their corresponding genes and homologues thereof in cell function, including their function in disease such as pre-eclampsia. For example, truncated or modified polypeptides may be introduced into a cell to disrupt the normal functions which occur in the cell.

ELABELA polypeptides, variants, homologues, fragments and derivatives disclosed here may also be used to diagnose, detect susceptibility to, treat, alleviate or prevent pre-eclampsia in an individual

The polypeptides may be introduced into the cell by in situ expression of the polypeptide from a recombinant expression vector (see elsewhere in this document). The expression vector optionally carries an inducible promoter to control the expression of the polypeptide.

The use of appropriate host cells, such as insect cells or mammalian cells, is expected to provide for such post-translational modifications (e.g. myristolation, glycosylation, truncation, lapidation and tyrosine, serine or threonine phosphorylation) as may be needed to confer optimal biological activity on recombinant expression products.

Such cell culture systems in which the ELABELA polypeptides, variants, homologues, fragments and derivatives disclosed here are expressed may be used in assay systems to identify candidate substances which interfere with or enhance the functions of the polypeptides in the cell.

We describe methods of making an ELABELA polypeptide or fragment thereof, the methods comprising: (a) expressing a nucleic acid encoding a sequence comprising CXXXRCXXXHSRVPFP (SEQ ID NO: 1) in a cell, wherein X is an/any amino acid residue, and wherein the polypeptide fragment maintains self-renewal, pluripotency, or both of a stem cell; or (b) using chemical synthesis to generate a synthetic polypeptide or fragment thereof comprising CXXXRCXXXHSRVPFP (SEQ ID NO: 1) or a fragment having the sequence of SEQ ID NO: 53, wherein X is an/any amino acid residue, and wherein the polypeptide fragment maintains self-renewal, pluripotency, or both of a stem cell.

The cell expressing the nucleic acid encoding a sequence comprising CXXXRCXXXHSRVPFP (SEQ ID NO: 1) may comprise a bacterial, fungal, or yeast cell.

The sequence comprising CXXXRCXXXHSRVPFP (SEQ ID NO: 1) may be selected from the group consisting of SEQ ID NOs: 2-36.

The ELABELA polypeptide or fragment thereof further may comprise a label. The label may comprise a radioisotope. The radioisotope may comprise ¹²⁵I.

The polypeptide or fragment thereof may be derivatized.

### ELABELA Nucleic Acids

The methods and compositions described here may make use of ELABELA polynucleotides, ELABELA nucleotides and ELABELA nucleic acids, as well as variants, homologues, derivatives and fragments of any of these, for the diagnosis, detection of susceptibility to, treatment, alleviation or prophylaxis of pre-eclampsia in an individual.

The terms “ELABELA polynucleotide”, “ELABELA nucleotide” and “ELABELA nucleic acid” may be used interchangeably, and should be understood to specifically include both cDNA and genomic ELABELA sequences. These terms are also intended to include a nucleic acid sequence capable of encoding an ELABELA polypeptide and/or a fragment, derivative, homologue or variant of this. These terms are also intended to include a nucleic acid sequence which is a fragment, derivative, homologue or variant of an ELABELA polypeptide having a specific sequence disclosed in this document, for example as set out in the sequence listings.

Where reference is made to an ELABELA nucleic acid, this should be taken as a reference to a nucleic acid sequence capable of encoding an ELABELA polypeptide. Such nucleic acids may encode ELABELA polypeptides comprising one or more biological activities of a native ELABELA polypeptide, such as maintaining self-renewal, pluripotency, or both of a stem cell.

For example, an ELABELA nucleic acid sequence may be capable of encoding a polypeptide comprising a sequence CXXXRCXXXHSRVPFP (SEQ ID NO: 1), in which X signifies an amino acid residue. The resulting encoded polypeptide sequence may comprise ELABELA activity, such as being capable of maintaining self-renewal and/or pluripotency of a stem cell.

An ELABELA nucleic acid may also be taken generally to refer to any member of the ELABELA family of nucleic acids.

ELABELA nucleic acids may for example be capable of encoding polypeptides comprising any of the sequences set out as SEQ ID NO: 2 to SEQ ID NO: 19. An ELABELA nucleic acid may be capable of encoding a polypeptide comprising a sequence CLQRRCMPLHSRVPFP (SEQ ID NO: 2).

Examples of ELABELA nucleic acids include those selected from the group consisting of SEQ ID NO: 37 to SEQ ID NO: 41 or SEQ ID NO: 42 to SEQ ID NO: 46. For example, a human ELABELA nucleic acid sequence having the sequence SEQ ID NO: 37 is disclosed.

Also included are any one or more of the nucleic acid sequences set out as “Other ELABELA nucleic acid sequences” elsewhere in this document.

For example, the ELABELA nucleic acid may comprise a human ELABELA sequence SEQ ID NO: 37.

ELABELA nucleic acids may be used for a variety of means, as described in this document. For example, ELABELA nucleic acids may be used treat an individual suffering from, or suspected to be suffering from pre-eclampsia, or to prevent such a condition or to alleviate any symptoms arising as a result of such a condition. ELABELA nucleic acids may also be used for the expression or production of ELABELA polypeptides. Other uses will be evident to the skilled reader, and are also encompassed in this document.

The term “polynucleotide”, as used in this document, generally refers to any polyribonucleotide or polydeoxribonucleotide, which may be unmodified RNA or DNA or modified RNA or DNA. “Polynucleotides” include, without limitation single- and double-stranded DNA, DNA that is a mixture of single- and double-stranded regions, single- and double-stranded RNA, and RNA that is mixture of single- and double-stranded regions, hybrid molecules comprising DNA and RNA that may be single-stranded or, more typically, double-stranded or a mixture of single- and double-stranded regions. In addition, “polynucleotide” refers to triple-stranded regions comprising RNA or DNA or both RNA and DNA. The term polynucleotide also includes DNAs or RNAs containing one or more modified bases and DNAs or RNAs with backbones modified for stability or for other reasons. “Modified” bases include, for example, tritylated bases and unusual bases such as inosine. A variety of modifications has been made to DNA and RNA; thus, “polynucleotide” embraces chemically, enzymatically or metabolically modified forms of polynucleotides as typically found in nature, as well as the chemical forms of DNA and RNA characteristic of viruses and cells. “Polynucleotide” also embraces relatively short polynucleotides, often referred to as oligonucleotides.

It will be understood by the skilled person that numerous nucleotide sequences can encode the same polypeptide as a result of the degeneracy of the genetic code.

As used herein, the term “nucleotide sequence” refers to nucleotide sequences, oligonucleotide sequences, polynucleotide sequences and variants, homologues, fragments and derivatives thereof (such as portions thereof). The nucleotide sequence may be DNA or RNA of genomic or synthetic or recombinant origin which may be double-stranded or single-stranded whether representing the sense or antisense strand or combinations thereof. The term nucleotide sequence may be prepared by use of recombinant DNA techniques (for example, recombinant DNA).

The term “nucleotide sequence” may mean DNA.

Other Nucleic Acids

We also provide nucleic acids which are fragments, homologues, variants or derivatives of ELABELA nucleic acids. The terms “variant”, “homologue”, “derivative” or “fragment” in relation to ELABELA nucleic acid include any substitution of, variation of, modification of, replacement of, deletion of or addition of one (or more) nucleic acids from or to the sequence of an ELABELA nucleotide sequence. Unless the context admits otherwise, references to “ELABELA” and “ELABELA nucleic acid”, “ELABELA nucleotide sequence” etc include references to such variants, homologues, derivatives and fragments of ELABELA.

The resultant nucleotide sequence may encode a polypeptide having any one or more ELABELA activity. The term “homologue” may be intended to cover identity with respect to structure and/or function such that the resultant nucleotide sequence encodes a polypeptide which has ELABELA activity. For example, a homologue etc of ELABELA may have a decreased expression level in cells from an individual suffering from pre-eclampsia compared to normal cells. With respect to sequence identity (i.e. similarity), there may be at least 70%, at least 75%, at least 85% or at least 90% sequence identity. There may be at least 95%, such as at least 98%, sequence identity to a relevant sequence such as any nucleic acid sequence shown in the sequence listings (e.g., an ELABELA sequence having SEQ ID NO: 37). These terms also encompass allelic variations of the sequences.

Variants, Derivatives and Homologues

ELABELA nucleic acid variants, fragments, derivatives and homologues may comprise DNA or RNA. They may be single-stranded or double-stranded. They may also be polynucleotides which include within them synthetic or modified nucleotides. A number of different types of modification to oligonucleotides are known in the art. These include methylphosphonate and phosphorothioate backbones, addition of acridine or polylysine chains at the 3′ and/or 5′ ends of the molecule. For the purposes of this document, it is to be understood that the polynucleotides may be modified by any method available in the art. Such modifications may be carried out in order to enhance the in vivo activity or life span of polynucleotides of interest.

Where the polynucleotide is double-stranded, both strands of the duplex, either individually or in combination, are encompassed by the methods and compositions described here. Where the polynucleotide is single-stranded, it is to be understood that the complementary sequence of that polynucleotide is also included.

The terms “variant”, “homologue” or “derivative” in relation to a nucleotide sequence include any substitution of, variation of, modification of, replacement of, deletion of or addition of one (or more) nucleic acid from or to the sequence. Said variant, homologues or derivatives may code for a polypeptide having biological activity. Such fragments, homologues, variants and derivatives of ELABELA may comprise modulated activity, as set out above.

As indicated above, with respect to sequence identity, a “homologue” may have at least 5% identity, at least 10% identity, at least 15% identity, at least 20% identity, at least 25% identity, at least 30% identity, at least 35% identity, at least 40% identity, at least 45% identity, at least 50% identity, at least 55% identity, at least 60% identity, at least 65% identity, at least 70% identity, at least 75% identity, at least 80% identity, at least 85% identity, at least 90% identity, or at least 95% identity to the relevant sequence, such as any nucleic acid sequence shown in the sequence listings (e.g., an ELABELA sequence having SEQ ID NO: 37).

There may be at least 95% identity, at least 96% identity, at least 97% identity, at least 98% identity or at least 99% identity. Nucleotide identity comparisons may be conducted as described above. A sequence comparison program which may be used is the GCG Wisconsin Bestfit program described above. The default scoring matrix has a match value of 10 for each identical nucleotide and −9 for each mismatch. The default gap creation penalty is −50 and the default gap extension penalty is −3 for each nucleotide.

Hybridisation

We further describe nucleotide sequences that are capable of hybridising selectively to any of the sequences presented herein, or any variant, fragment or derivative thereof, or to the complement of any of the above. Nucleotide sequences may be at least 5, 10, or 15 nucleotides in length, such as at least 20, 30, 40 or 50 nucleotides in length.

The term “hybridization” as used herein shall include “the process by which a strand of nucleic acid joins with a complementary strand through base pairing” as well as the process of amplification as carried out in polymerase chain reaction technologies.

Polynucleotides capable of selectively hybridising to the nucleotide sequences presented herein, or to their complement, may be at least 40% homologous, at least 45% homologous, at least 50% homologous, at least 55% homologous, at least 60% homologous, at least 65% homologous, at least 70% homologous, at least 75% homologous, at least 80% homologous, at least 85% homologous, at least 90% homologous, or at least 95% homologous to the corresponding nucleotide sequences presented herein, such as any nucleic acid sequence shown in the sequence listings (e.g., an ELABELA sequence having SEQ ID NO: 37). Such polynucleotides may be generally at least 70%, at least 80 or 90% or at least 95% or 98% homologous to the corresponding nucleotide sequences over a region of at least 5, 10, 15 or 20, such as at least 25 or 30, for instance at least 40, 60 or 100 or more contiguous nucleotides.

The term “selectively hybridizable” means that the polynucleotide used as a probe is used under conditions where a target polynucleotide is found to hybridize to the probe at a level significantly above background. The background hybridization may occur because of other polynucleotides present, for example, in the cDNA or genomic DNA library being screening. In this event, background implies a level of signal generated by interaction between the probe and a non-specific DNA member of the library which is less than 10 fold, such as less than 100 fold as intense as the specific interaction observed with the target DNA. The intensity of interaction may be measured, for example, by radiolabelling the probe, e.g. with ³²P or ³³P or with non-radioactive probes (e.g., fluorescent dyes, biotin or digoxigenin).

Hybridization conditions are based on the melting temperature (Tm) of the nucleic acid binding complex, as taught in Berger and Kimmel (1987, Guide to Molecular Cloning Techniques, Methods in Enzymology, Vol 152, Academic Press, San Diego Calif.), and confer a defined “stringency” as explained elsewhere in this document.

Maximum stringency typically occurs at about Tm-5° C. (5° C. below the Tm of the probe); high stringency at about 5° C. to 10° C. below Tm; intermediate stringency at about 10° C. to 20° C. below Tm; and low stringency at about 20° C. to 25° C. below Tm. As will be understood by those of skill in the art, a maximum stringency hybridization can be used to identify or detect identical polynucleotide sequences while an intermediate (or low) stringency hybridization can be used to identify or detect similar or related polynucleotide sequences.

We provide nucleotide sequences that may be able to hybridise to the ELABELA nucleic acids, fragments, variants, homologues or derivatives under stringent conditions (e.g. 65° C. and 0.1×SSC (1×SSC=0.15 M NaCl, 0.015 M Na₃Citrate pH 7.0)).

Generation of Homologues, Variants and Derivatives

Polynucleotides which are not 100% identical to the relevant sequences (e.g., a human ELABELA sequence having SEQ ID NO: 37) but which are also included, as well as homologues, variants and derivatives of ELABELA can be obtained in a number of ways. Other variants of the sequences may be obtained for example by probing DNA libraries made from a range of individuals, for example individuals from different populations. For example, ELABELA homologues may be identified from other individuals, or other species. Further recombinant ELABELA nucleic acids and polypeptides may be produced by identifying corresponding positions in the homologues, and synthesising or producing the molecule as described elsewhere in this document.

In addition, other viral/bacterial, or cellular homologues of ELABELA, particularly cellular homologues found in mammalian cells (e.g. rat, mouse, bovine and primate cells), may be obtained and such homologues and fragments thereof in general will be capable of selectively hybridising to human ELABELA. Such homologues may be used to design non-human ELABELA nucleic acids, fragments, variants and homologues. Mutagenesis may be carried out by means known in the art to produce further variety.

Sequences of ELABELA homologues may be obtained by probing cDNA libraries made from or genomic DNA libraries from other animal species, and probing such libraries with probes comprising all or part of any of the ELABELA nucleic acids, fragments, variants and homologues, or other fragments of ELABELA under conditions of medium to high stringency.

Similar considerations apply to obtaining species homologues and allelic variants of the polypeptide or nucleotide sequences disclosed here.

Variants and strain/species homologues may also be obtained using degenerate PCR which will use primers designed to target sequences within the variants and homologues encoding conserved amino acid sequences within the sequences of the ELABELA nucleic acids. Conserved sequences can be predicted, for example, by aligning the amino acid sequences from several variants/homologues. Sequence alignments can be performed using computer software known in the art. For example the GCG Wisconsin PileUp program is widely used.

The primers used in degenerate PCR will contain one or more degenerate positions and will be used at stringency conditions lower than those used for cloning sequences with single sequence primers against known sequences. It will be appreciated by the skilled person that overall nucleotide homology between sequences from distantly related organisms is likely to be very low and thus in these situations degenerate PCR may be the method of choice rather than screening libraries with labelled fragments the ELABELA sequences.

In addition, homologous sequences may be identified by searching nucleotide and/or protein databases using search algorithms such as the BLAST suite of programs.

Alternatively, such polynucleotides may be obtained by site directed mutagenesis of characterised sequences, for example, ELABELA nucleic acids, or variants, homologues, derivatives or fragments thereof. This may be useful where for example silent codon changes are required to sequences to optimise codon preferences for a particular host cell in which the polynucleotide sequences are being expressed. Other sequence changes may be desired in order to introduce restriction enzyme recognition sites, or to alter the property or function of the polypeptides encoded by the polynucleotides.

The polynucleotides described here may be used to produce a primer, e.g. a PCR primer, a primer for an alternative amplification reaction, a probe e.g. labelled with a revealing label by conventional means using radioactive or non-radioactive labels, or the polynucleotides may be cloned into vectors. Such primers, probes and other fragments will be at least 8, 9, 10, or 15, such as at least 20, for example at least 25, 30 or 40 nucleotides in length, and are also encompassed by the term “polynucleotides” as used herein.

Polynucleotides such as a DNA polynucleotides and probes may be produced recombinantly, synthetically, or by any means available to those of skill in the art. They may also be cloned by standard techniques.

In general, primers will be produced by synthetic means, involving a step wise manufacture of the desired nucleic acid sequence one nucleotide at a time. Techniques for accomplishing this using automated techniques are readily available in the art.

Primers comprising fragments of ELABELA are particularly useful in the methods of detection of ELABELA expression, such as down-regulation of ELABELA expression, for example, as associated with pre-eclampsia. Suitable primers for amplification of ELABELA may be generated from any suitable stretch of ELABELA. Primers which may be used include those capable of amplifying a sequence of ELABELA which is specific.

Although ELABELA primers may be provided on their own, they are most usefully provided as primer pairs, comprising a forward primer and a reverse primer.

Longer polynucleotides will generally be produced using recombinant means, for example using a PCR (polymerase chain reaction) cloning techniques. This will involve making a pair of primers (e.g. of about 15 to 30 nucleotides), bringing the primers into contact with mRNA or cDNA obtained from an animal or human cell, performing a polymerase chain reaction under conditions which bring about amplification of the desired region, isolating the amplified fragment (e.g. by purifying the reaction mixture on an agarose gel) and recovering the amplified DNA. The primers may be designed to contain suitable restriction enzyme recognition sites so that the amplified DNA can be cloned into a suitable cloning vector.

Polynucleotides or primers may carry a revealing label. Suitable labels include radioisotopes such as ³²P or ³⁵S, digoxigenin, fluorescent dyes, enzyme labels, or other protein labels such as biotin. Such labels may be added to polynucleotides or primers and may be detected using by techniques known per se. Polynucleotides or primers or fragments thereof labelled or unlabeled may be used by a person skilled in the art in nucleic acid-based tests for detecting or sequencing polynucleotides in the human or animal body.

Such tests for detecting generally comprise bringing a biological sample containing DNA or RNA into contact with a probe comprising a polynucleotide or primer under hybridising conditions and detecting any duplex formed between the probe and nucleic acid in the sample. Such detection may be achieved using techniques such as PCR or by immobilising the probe on a solid support, removing nucleic acid in the sample which is not hybridised to the probe, and then detecting nucleic acid which has hybridised to the probe. Alternatively, the sample nucleic acid may be immobilised on a solid support, and the amount of probe bound to such a support can be detected. Suitable assay methods of this and other formats can be found in for example WO89/03891 and WO90/13667.

Tests for sequencing nucleotides, for example, the ELABELA nucleic acids, involve bringing a biological sample containing target DNA or RNA into contact with a probe comprising a polynucleotide or primer under hybridising conditions and determining the sequence by, for example the Sanger dideoxy chain termination method (see Sambrook et al.).

Such a method generally comprises elongating, in the presence of suitable reagents, the primer by synthesis of a strand complementary to the target DNA or RNA and selectively terminating the elongation reaction at one or more of an A, C, G or T/U residue; allowing strand elongation and termination reaction to occur; separating out according to size the elongated products to determine the sequence of the nucleotides at which selective termination has occurred. Suitable reagents include a DNA polymerase enzyme, the deoxynucleotides dATP, dCTP, dGTP and dTTP, a buffer and ATP. Dideoxynucleotides are used for selective termination.

### Detection and Diagnostic Methods

Detection of Expression of ELABELA

We describe methods of detecting the expression of ELABELA, including ELABELA polypeptides, ELABELA nucleic acids and variants, homologues, derivatives and fragments thereof, etc. Such methods are useful for the diagnosis or detection of susceptibility to, pre-eclampsia in an individual.

ELABELA expression may be detected as a means to determine the quantity of ELABELA or its activity. ELABELA expression may be detected in or of a cell. Detection of ELABELA expression may also be conducted on a sample comprising a cell tissue, an organ or part or all of an organism.

Expression of ELABELA in an pre-eclampsia may be modulated, such as down-regulated when compared to normal tissue. Accordingly, we provide for a method of diagnosis of pre-eclampsia, comprising detecting modulation of expression of ELABELA, such as modulation of down-regulation of expression of ELABELA in a cell or tissue of an individual.

Detection of ELABELA expression, activity or amount may be used to provide a method of determining the state of a cell. Thus, a cell of interest may be one with high levels of ELABELA expression, activity or amount compared to a normal cell. Similarly, a cell of interest may be one with low levels ELABELA expression, activity or amount compared to a normal cell.

Detection of ELABELA may also be used to determine whether a cell is a cell of interest. Thus, a high level of ELABELA expression, amount or activity of ELABELA in the cell may be detected. Similarly, a low level of ELABELA expression, amount or activity may also be detected in a cell.

It will be appreciated that if the level of ELABELA varies with the aggressiveness of pre-eclampsia, that detection of ELABELA expression, amount or activity may also be used to predict a survival rate of an individual with pre-eclampsia, i.e., lower levels of ELABELA indicating a lower survival rate or probability and higher levels of ELABELA indicating a higher survival rate or probability, both as compared to individuals or cognate populations with normal levels of ELABELA. Detection of expression, amount or activity of ELABELA may therefore be used as a method of prognosis of an individual with pre-eclampsia.

Detection of ELABELA expression, amount or level may be used to determine the likelihood of success of a particular therapy in an individual with pre-eclampsia.

The diagnostic methods described in this document may be combined with the therapeutic methods described. Thus, we provide for a method of treatment, prophylaxis or alleviation of pre-eclampsia in an individual, the method comprising detecting modulation of expression, amount or activity of ELABELA in a cell of the individual and administering an appropriate therapy to the individual based on the aggressiveness of the pre-eclampsia. The therapy may comprise ELABELA or an ELABELA agonist as described elsewhere.

The presence and quantity of ELABELA polypeptides and nucleic acids may be detected in a sample as described in further detail elsewhere in this document. Thus, pre-eclampsia may be diagnosed by methods comprising determining from a sample derived from a subject an abnormally decreased expression, amount or activity of the ELABELA polypeptide or ELABELA mRNA.

The sample may comprise a cell or tissue sample from an organism or individual suffering or suspected to be suffering from a disease associated with increased, reduced or otherwise abnormal ELABELA expression, amount or activity, including spatial or temporal changes in level or pattern of expression, amount or activity (such as pre-eclampsia). The level or pattern of expression, amount or activity of ELABELA in an organism suffering from or suspected to be suffering from such a disease including pre-eclampsia may be usefully compared with the level or pattern of expression, amount or activity in a normal organism as a means of diagnosis of disease.

The sample may comprise a cell or tissue sample from an individual suffering or suspected to be suffering from pre-eclampsia, such as a tissue or cell sample of any of those tissues or cells.

In some embodiments, an decreased level of expression, amount or activity of ELABELA is detected in the sample. The level of ELABELA may be decreased to a significant extent when compared to normal cells, or cells from an individual known not to be suffering from pre-eclampsia. Such cells may be obtained from the individual being tested, or another individual, such as those matched to the tested individual by age, weight, lifestyle, etc.

In some embodiments, the level of expression, amount or activity of ELABELA is decreased by 10%, 20%, 30% or 40% or more. In some embodiments, the level of expression, amount or activity of ELABELA is decreased by 45% or more, such as 50% or more, for example as judged by cDNA hybridisation.

The expression, amount or activity of ELABELA may be detected in a number of ways, as known in the art, and as described in further detail elsewhere in this document. Typically, the amount of ELABELA in a sample of tissue from an individual is measured, and compared with a sample from an unaffected individual. Both ELABELA nucleic acid, as well as ELABELA polypeptide levels may be measured.

Detection of the amount, activity or expression of ELABELA may be used to grade pre-eclampsia.

Levels of ELABELA gene expression may be determined using a number of different techniques.

Measuring Expression of ELABELA at the RNA Level

ELABELA gene expression can be detected at the RNA level.

In one embodiment therefore, we disclose a method of detecting the presence of a nucleic acid comprising an ELABELA nucleic acid in a sample, by contacting the sample with at least one nucleic acid probe which is specific for the ELABELA nucleic acid and monitoring said sample for the presence of the ELABELA nucleic acid. For example, the nucleic acid probe may specifically bind to the ELABELA nucleic acid, or a portion of it, and binding between the two detected; the presence of the complex itself may also be detected.

Thus, in one embodiment, the amount of ELABELA nucleic acid in the form of ELABELA mRNA may be measured in a sample. ELABELA mRNA may be assayed by in situ hybridization, Northern blotting and reverse transcriptase-polymerase chain reaction. Nucleic acid sequences may be identified by in situ hybridization, Southern blotting, single strand conformational polymorphism, PCR amplification and DNA-chip analysis using specific primers. (Kawasaki, 1990; Sambrook, 1992; Lichter et al, 1990; Orita et al, 1989; Fodor et al., 1993; Pease et al., 1994).

ELABELA RNA may be extracted from cells using RNA extraction techniques including, for example, using acid phenol/guanidine isothiocyanate extraction (RNAzol B; Biogenesis), or RNeasy RNA preparation kits (Qiagen). Typical assay formats utilising ribonucleic acid hybridisation include nuclear run-on assays, RT-PCR and RNase protection assays (Melton et al., Nuc. Acids Res. 12:7035. Methods for detection which can be employed include radioactive labels, enzyme labels, chemiluminescent labels, fluorescent labels and other suitable labels.

Each of these methods allows quantitative determinations to be made, and are well known in the art. Decreased or increased ELABELA expression, amount or activity can therefore be measured at the RNA level using any of the methods well known in the art for the quantitation of polynucleotides. Any suitable probe from an ELABELA sequence, for example, any portion of a suitable human ELABELA sequence may be used as a probe. Sequences for designing ELABELA probes may include a sequence having SEQ ID NO: 37 to 41, or a portion thereof.

Typically, RT-PCR is used to amplify RNA targets. In this process, the reverse transcriptase enzyme is used to convert RNA to complementary DNA (cDNA) which can then be amplified to facilitate detection.

Many DNA amplification methods are known, most of which rely on an enzymatic chain reaction (such as a polymerase chain reaction, a ligase chain reaction, or a self-sustained sequence replication) or from the replication of all or part of the vector into which it has been cloned.

Many target and signal amplification methods have been described in the literature, for example, general reviews of these methods in Landegren, U. et al., Science 242:229-237 (1988) and Lewis, R., Genetic Engineering News 10:1, 54-55 (1990).

For example, the polymerase chain reaction may be employed to detect ELABELA mRNA.

The “polymerase chain reaction” or “PCR” is a nucleic acid amplification method described inter alia in U.S. Pat. Nos. 4,683,195 and 4,683,202. PCR can be used to amplify any known nucleic acid in a diagnostic context (Mok et al., 1994, Gynaecologic Oncology 52:247-252). Self-sustained sequence replication (3SR) is a variation of TAS, which involves the isothermal amplification of a nucleic acid template via sequential rounds of reverse transcriptase (RT), polymerase and nuclease activities that are mediated by an enzyme cocktail and appropriate oligonucleotide primers (Guatelli et al., 1990, Proc. Natl. Acad. Sci. USA 87:1874). Ligation amplification reaction or ligation amplification system uses DNA ligase and four oligonucleotides, two per target strand. This technique is described by Wu, D. Y. and Wallace, R. B., 1989, Genomics 4:560. In the QI3 Replicase technique, RNA replicase for the bacteriophage QI3, which replicates single-stranded RNA, is used to amplify the target DNA, as described by Lizardi et al., 1988, Bio/Technology 6:1197.

A PCR procedure basically involves: (1) treating extracted DNA to form single-stranded complementary strands; (2) adding a pair of oligonucleotide primers, wherein one primer of the pair is substantially complementary to part of the sequence in the sense strand and the other primer of each pair is substantially complementary to a different part of the same sequence in the complementary antisense strand; (3) annealing the paired primers to the complementary sequence; (4) simultaneously extending the annealed primers from a 3′ terminus of each primer to synthesize an extension product complementary to the strands annealed to each primer wherein said extension products after separation from the complement serve as templates for the synthesis of an extension product for the other primer of each pair; (5) separating said extension products from said templates to produce single-stranded molecules; and (6) amplifying said single-stranded molecules by repeating at least once said annealing, extending and separating steps.

Reverse transcription-polymerase chain reaction (RT-PCR) may be employed. Quantitative RT-PCR may also be used. Such PCR techniques are well known in the art, and may employ any suitable primer from an ELABELA sequence.

Alternative amplification technology can also be exploited. For example, rolling circle amplification (Lizardi et al., 1998, Nat Genet 19:225) is an amplification technology available commercially (RCAT™) which is driven by DNA polymerase and can replicate circular oligonucleotide probes with either linear or geometric kinetics under isothermal conditions. A further technique, strand displacement amplification (SDA; Walker et al., 1992, Proc. Natl. Acad. Sci. USA 80:392) begins with a specifically defined sequence unique to a specific target.

Measuring Expression of ELABELA at the Polypeptide Level

ELABELA expression can be detected at the polypeptide level.

In a further embodiment, therefore, ELABELA expression, amount or activity may be detected by detecting the presence or amount of ELABELA polypeptide in a sample. This may be achieved by using molecules which bind to ELABELA polypeptide. Suitable molecules/agents which bind either directly or indirectly to the ELABELA polypeptide in order to detect its presence include naturally occurring molecules such as peptides and proteins, for example antibodies, or they may be synthetic molecules.

Thus, we disclose a method of detecting the presence of an ELABELA polypeptide by contacting a cell sample with an antibody capable of binding the polypeptide and monitoring said sample for the presence of the polypeptide.

For example, the ELABELA polypeptide may be detected using an anti-ELABELA antibody. Such antibodies may be made by means known in the art (such as described in International Patent Publication WO 2015/084264).

Detection of ELABELA may conveniently be achieved by monitoring the presence of a complex formed between the antibody and the ELABELA polypeptide, or monitoring the binding between the polypeptide and the antibody. Methods of detecting binding between two entities are known in the art, and include FRET (fluorescence resonance energy transfer), surface plasmon resonance, etc.

Standard laboratory techniques such as immunoblotting as described above can be used to detect altered levels of ELABELA protein, as compared with untreated cells in the same cell population.

Gene expression may also be determined by detecting changes in post-translational processing of ELABELA polypeptides or post-transcriptional modification of ELABELA nucleic acids. For example, differential phosphorylation of ELABELA polypeptides, the cleavage of ELABELA polypeptides or alternative splicing of ELABELA RNA, and the like may be measured. Levels of expression of gene products such as ELABELA polypeptides, as well as their post-translational modification, may be detected using proprietary protein assays or techniques such as 2D polyacrylamide gel electrophoresis.

Assay techniques that can be used to determine levels of ELABELA protein in a sample derived from a host are well-known to those of skill in the art. Antibodies can be assayed for immunospecific binding by any method known in the art.

The immunoassays which can be used include but are not limited to competitive and non-competitive assay systems using techniques such as western blots, radioimmunoassays, ELISA, sandwich immunoassays, immunoprecipitation assays, precipitin reactions, gel diffusion precipitin reactions, immunodiffusion assays, agglutination assays, complement-fixation assays, immunoradiometric assays, fluorescent immunoassays and protein A immunoassays. Such assays are routine in the art (see, for example, Ausubel et al., eds, 1994, Current Protocols in Molecular Biology, Vol. 1, John Wiley & Sons, Inc., New York, which is incorporated by reference herein in its entirety).

We describe immunoassay kits for measuring or detecting ELABELA expression for the diagnosis or detection of susceptibility to pre-eclampsia in an individual. The immunoassay kits may comprise: a coating antigen; (b) one or more isolated antibodies or antigen-binding fragments thereof that specifically binds to one or more of the following: (i) a polypeptide comprising the sequence CMPLHSRVPFP (SEQ ID NO: 52); (ii) a polypeptide comprising the sequence QRPVNLTMRRKLRKHNC (SEQ ID NO: 53); (iii) a polypeptide comprising the sequence QRPVNLTMRRKLRKHNCLQRRCMPLHSRVPFP (SEQ ID NO: 2); (iv) a polypeptide comprising the sequence of any of SEQID NOs: 1 to 36; and (c) instructions for use.

The isolated antibodies or antigen-binding fragments may be labelled, such as with a radiolabel, for example ¹²⁵I.

The immunoassay kit may further comprise an enzyme labelled reagent, a secondary antibody capable of specifically binding to the isolated antibodies or antigen-binding fragments, a solid substrate, or any combination of these.

The specimen may be assayed for polypeptides/proteins by immunohistochemical and immunocytochemical staining (see generally Stites and Terr, Basic and Clinical Immunology, Appleton and Lange, 1994), ELISA, RIA, immunoblots, Western blotting, immunoprecipitation, functional assays and protein truncation test. Other assay methods include radioimmunoassays, competitive-binding assays, Western Blot analysis and ELISA assays.

ELISA assays are well known to those skilled in the art. Both polyclonal and monoclonal antibodies may be used in the assays. Where appropriate other immunoassays, such as radioimmunoassays (MA) may be used as are known to those in the art. Available immunoassays are extensively described in the patent and scientific literature. See, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521 as well as Sambrook et al, 1992.

Diagnostic Kits

We also provide diagnostic kits for detecting pre-eclampsia in an individual, or susceptibility to pre-eclampsia in an individual.

The diagnostic kit may comprise means for detecting expression, amount or activity of ELABELA in the individual, by any means as described in this document. The diagnostic kit may therefore comprise any one or more of the following: an ELABELA polynucleotide or a fragment thereof; a complementary nucleotide sequence to ELABELA nucleic acid or a fragment thereof; an ELABELA polypeptide or a fragment thereof, or an antibody to an ELABELA, such as comprising an anti-ELABELA antibody against ELABELA, e.g., an anti-peptide antibody human ELABELA antibody.

The diagnostic kit may comprise instructions for use, or other indicia. The diagnostic kit may further comprise means for treatment or prophylaxis of pre-eclampsia, such as any of the compositions described in this document, or any means known in the art for treating such pre-eclampsia. In particular, the diagnostic kit may comprise ELABELA polypeptide or nucleic acid. The diagnostic kit may comprise a therapeutic drug.

Immunoassays

We describe immunoassay kits for measuring or detecting ELABELA expression, the immunoassay kits comprising: (a) a coating antigen comprising one or more isolated antibodies or antigen-binding fragments thereof that specifically binds to one or more of the following: (i) a polypeptide comprising the sequence CMPLHSRVPFP (SEQ ID NO: 52); (ii) a polypeptide comprising the sequence QRPVNLTMRRKLRKHNC (SEQ ID NO: 53); (iii) a polypeptide comprising the sequence QRPVNLTMRRKLRKHNCLQRRCMPLHSRVPFP (SEQ ID NO: 2); or (iv) an ELABELA polypeptide comprising the sequence of any of SEQ ID NOs: 1-36; and (b) instructions for using said coating antigen.

The isolated antibodies or antigen-binding fragments thereof may be labelled.

The kit may further comprise an enzyme labelled reagent, a secondary antibody that specifically binds to the isolated antibodies or antigen-binding fragments, a solid substrate, or any combination thereof.

### Prophylactic and Therapeutic Methods

We disclose methods of treating an abnormal condition, such as pre-eclampsia, related to insufficient or excessive amounts of ELABELA expression or activity. Methods of preventing pre-eclampsia (i.e., prophylaxis) also suitably employ the same or similar approaches.

In general terms, our methods involve manipulation of cells, by modulating (such as up-regulating) the expression, amount or activity of ELABELA in the cell. A step of detecting modulated ELABELA expression, amount or activity in a cell may be conducted before or after the manipulation step. The detection step may detect down-regulated ELABELA expression, amount or activity. Any of the methods of modulating or up-regulating ELABELA, as described in detail elsewhere in this document, may be used.

The method may comprise exposing the cell, organism or individual to ELABELA polypeptide. This is described in further detail below.

Pre-eclampsia is defined as being “treated” if a condition associated with the disease—such as pre-eclampsia—is significantly inhibited (i.e., by 50% or more) relative to controls. The inhibition may be by at least 75% relative to controls, such as by 90%, by 95% or 100% relative to controls. By the term “treatment” we mean to also include prophylaxis or alleviation of pre-eclampsia.

### Pharmaceutical Compositions and Administration

Methods of treatment of pre-eclampsia may comprise administering ELABELA, including an ELABELA nucleic acid, polypeptide, fragment, homologue, variant or derivative thereof, modulator, agonist, a structurally related compound, or an acidic salt of either, to an individual in need of treatment thereof.

In the following discussion, ELABELA, including an ELABELA nucleic acid, polypeptide, fragment, homologue, variant or derivative thereof, modulator, agonist, a structurally related compound, or an acidic salt of either will be referred to as an “ELABELA agent”.

While it is possible for the ELABELA agent to be administered alone, the active ingredient may be formulated as a pharmaceutical formulation.

We therefore also disclose pharmaceutical compositions comprising an ELABELA agent. Such pharmaceutical compositions are useful for delivery of the ELABELA agent such as in the form of a composition as described, to an individual for the treatment or alleviation of symptoms as described.

A pharmaceutical composition in the context of the present document is a composition of matter comprising at least an ELABELA agent as an active ingredient.

The pharmaceutical formulations comprise an effective amount of the ELABELA agent together with one or more pharmaceutically-acceptable carriers. An “effective amount” is the amount sufficient to alleviate at least one symptom of a disease such as pre-eclampsia as described.

The effective amount will vary depending upon the particular disease or syndrome to be treated or alleviated (including pre-eclampsia), as well as other factors including the age and weight of the patient, how advanced the disease etc state is, the general health of the patient, the severity of the symptoms, and whether the ELABELA agent is being administered alone or in combination with other therapies.

Suitable pharmaceutically acceptable carriers are well known in the art and vary with the desired form and mode of administration of the pharmaceutical formulation. For example, they can include diluents or excipients such as fillers, binders, wetting agents, disintegrators, surface-active agents, lubricants and the like. Typically, the carrier is a solid, a liquid or a vaporizable carrier, or a combination thereof. Each carrier should be “acceptable” in the sense of being compatible with the other ingredients in the formulation and not injurious to the patient. The carrier should be biologically acceptable without eliciting an adverse reaction (e.g. immune response) when administered to the host.

The active ingredient(s) of a pharmaceutical composition is contemplated to exhibit therapeutic activity, for example, in the alleviation of pre-eclampsia. Dosage regimes may be adjusted to provide the optimum therapeutic response. For example, several divided doses may be administered daily or the dose may be proportionally reduced as indicated by the exigencies of the therapeutic situation.

The active compound may be administered in a convenient manner such as by the oral, intravenous (where water soluble), intramuscular, subcutaneous, intranasal, intradermal or suppository routes or implanting (e.g. using slow release molecules). Depending on the route of administration, the active ingredient may be required to be coated in a material to protect said ingredients from the action of enzymes, acids and other natural conditions which may inactivate said ingredient.

The ELABELA agent may be administered alone, or in combination with other therapeutic agents. Other therapeutic agents suitable for use herein are any compatible drugs that are effective for the intended purpose, or drugs that are complementary to the agent formulation. The formulation utilized in a combination therapy may be administered simultaneously, or sequentially with other treatment, such that a combined effect is achieved.

Oral Administration

In some embodiments, the ELABELA agent is provided as an oral composition and administered accordingly. The dosage of the ELABELA agent may be between about 1 mg/day to about 10 mg/day.

The pharmaceutical composition can be administered in an oral formulation in the form of tablets, capsules or solutions. An effective amount of the oral formulation is administered to patients 1 to 3 times daily until the symptoms of the disease, e.g., pre-eclampsia, alleviated.

The effective amount of agent depends on the age, weight and condition of a patient. In general, the daily oral dose of agent is less than 1200 mg, and more than 100 mg. The daily oral dose may be about 300-600 mg. Oral formulations are conveniently presented in a unit dosage form and may be prepared by any method known in the art of pharmacy. The composition may be formulated together with a suitable pharmaceutically acceptable carrier into any desired dosage form. Typical unit dosage forms include tablets, pills, powders, solutions, suspensions, emulsions, granules, capsules, suppositories. In general, the formulations are prepared by uniformly and intimately bringing into association the agent composition with liquid carriers or finely divided solid carriers or both, and as necessary, shaping the product. The active ingredient can be incorporated into a variety of basic materials in the form of a liquid, powder, tablets or capsules to give an effective amount of active ingredient to treat the disease such as pre-eclampsia.

The composition may be suitably orally administered, for example, with an inert diluent or with an assimilable edible carrier, or it may be enclosed in hard or soft shell gelatin capsules, or it may be compressed into tablets, or it may be incorporated directly with the food of the diet. For oral therapeutic administration, the active compound may be incorporated with excipients and used in the form of ingestible tablets, buccal tablets, troches, capsules, elixirs, suspensions, syrups, wafers, and the like. The amount of active compound in such therapeutically useful compositions in such that a suitable dosage will be obtained.

The tablets, troches, pills, capsules and the like may also contain the following: a binder such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, lactose or saccharin may be added or a flavouring agent such as peppermint, oil of wintergreen, or cherry flavouring. When the dosage unit form is a capsule, it may contain, in addition to materials of the above type, a liquid carrier.

Various other materials may be present as coatings or to otherwise modify the physical form of the dosage unit. For instance, tablets, pills, or capsules may be coated with shellac, sugar or both. A syrup or elixir may contain the active compound, sucrose as a sweetening agent, methyl and propylparabens as preservatives, a dye and flavouring such as cherry or orange flavour. Of course, any material used in preparing any dosage unit form should be pharmaceutically pure and substantially non-toxic in the amounts employed. In addition, the active compound may be incorporated into sustained-release preparations and formulations.

Injectable or Intravenous Administration

In some embodiments, the ELABELA agent is provided as an injectable or intravenenous composition and administered accordingly. The dosage of the ELABELA agent may be between about 5 mg/kg/2 weeks to about 10 mg/kg/2 weeks. The ELABELA agent may be provided in a dosage of between 10-300 mg/day, such as at least 30 mg/day, less than 200 mg/day or between 30 mg/day to 200 mg/day.

The pharmaceutical forms suitable for injectable use include sterile aqueous solutions (where water soluble) or dispersions and sterile powders for the extemporaneous preparation of sterile injectable solutions or dispersion. In all cases the form must be sterile and must be fluid to the extent that easy syringability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyetheylene gloycol, and the like), suitable mixtures thereof, and vegetable oils. The proper fluidity can be maintained, for example, by the use of a coating such as lecithin, by the maintenance of the required particle size in the case of dispersion and by the use of superfactants.

Topical Administration

The pharmaceutical compositions disclosed here include those suitable for topical and oral administration. Topical formulations may be used where the tissue affected is primarily the skin or epidermis.

The topical formulations include those pharmaceutical forms in which the composition is applied externally by direct contact with the skin surface to be treated. A conventional pharmaceutical form for topical application includes a soak, an ointment, a cream, a lotion, a paste, a gel, a stick, a spray, an aerosol, a bath oil, a solution and the like. Topical therapy is delivered by various vehicles, the choice of vehicle can be important and generally is related to whether an acute or chronic disease is to be treated. As an example, an acute skin proliferation disease generally is treated with aqueous drying preparations, whereas chronic skin proliferation disease is treated with hydrating preparations. Soaks are the easiest method of drying acute moist eruptions. Lotions (powder in water suspension) and solutions (medications dissolved in a solvent) are ideal for hairy and intertriginous areas. Ointments or water-in-oil emulsions, are the most effective hydrating agents, appropriate for dry scaly eruptions, but are greasy and depending upon the site of the lesion sometimes undesirable. As appropriate, they can be applied in combination with a bandage, particularly when it is desirable to increase penetration of the agent composition into a lesion. Creams or oil-in-water emulsions and gels are absorbable and are the most cosmetically acceptable to the patient. (Guzzo et al, in Goodman & Gilman's Pharmacological Basis of Therapeutics, 9th Ed., p. 1593-15950 (1996)). Cream formulations generally include components such as petroleum, lanolin, polyethylene glycols, mineral oil, glycerin, isopropyl palmitate, glyceryl stearate, cetearyl alcohol, tocopheryl acetate, isopropyl myristate, lanolin alcohol, simethicone, carbomen, methylchlorisothiazolinone, methylisothiazolinone, cyclomethicone and hydroxypropyl methylcellulose, as well as mixtures thereof.

Other formulations for topical application include shampoos, soaps, shake lotions, and the like, particularly those formulated to leave a residue on the underlying skin, such as the scalp (Arndt et al, in Dermatology In General Medicine 2:2838 (1993)).

In general, the concentration of the composition in the topical formulation is in an amount of about 0.5 to 50% by weight of the composition, such as about 1 to 30%, about 2-20%, or about 5-10%. The concentration used can be in the upper portion of the range initially, as treatment continues, the concentration can be lowered or the application of the formulation may be less frequent. Topical applications are often applied twice daily. However, once-daily application of a larger dose or more frequent applications of a smaller dose may be effective. The stratum corneum may act as a reservoir and allow gradual penetration of a drug into the viable skin layers over a prolonged period of time.

In a topical application, a sufficient amount of active ingredient must penetrate a patient's skin in order to obtain a desired pharmacological effect. It is generally understood that the absorption of drug into the skin is a function of the nature of the drug, the behaviour of the vehicle, and the skin. Three major variables account for differences in the rate of absorption or flux of different topical drugs or the same drug in different vehicles; the concentration of drug in the vehicle, the partition coefficient of drug between the stratum corneum and the vehicle and the diffusion coefficient of drug in the stratum corneum. To be effective for treatment, a drug must cross the stratum corneum which is responsible for the barrier function of the skin. In general, a topical formulation which exerts a high in vitro skin penetration is effective in vivo. Ostrenga et al (J. Pharm. Sci., 60:1175-1179 (1971) demonstrated that in vivo efficacy of topically applied steroids was proportional to the steroid penetration rate into dermatomed human skin in vitro.

A skin penetration enhancer which is dermatologically acceptable and compatible with the agent can be incorporated into the formulation to increase the penetration of the active compound(s) from the skin surface into epidermal keratinocytes. A skin enhancer which increases the absorption of the active compound(s) into the skin reduces the amount of agent needed for an effective treatment and provides for a longer lasting effect of the formulation. Skin penetration enhancers are well known in the art. For example, dimethyl sulfoxide (U.S. Pat. No. 3,711,602); oleic acid, 1,2-butanediol surfactant (Cooper, J. Pharm. Sci., 73:1153-1156 (1984)); a combination of ethanol and oleic acid or oleyl alcohol (EP 267,617), 2-ethyl-1,3-hexanediol (WO 87/03490); decyl methyl sulphoxide and Azone® (Hadgraft, Eur. J. Drug. Metab. Pharmacokinet, 21:165-173 (1996)); alcohols, sulphoxides, fatty acids, esters, Azone®, pyrrolidones, urea and polyoles (Kalbitz et al, Pharmazie, 51:619-637 (1996));

Terpenes such as 1,8-cineole, menthone, limonene and nerolidol (Yamane, J. Pharmacy & Pharmocology, 47:978-989 (1995)); Azone® and Transcutol (Harrison et al, Pharmaceutical Res. 13:542-546 (1996)); and oleic acid, polyethylene glycol and propylene glycol (Singh et al, Pharmazie, 51:741-744 (1996)) are known to improve skin penetration of an active ingredient.

Levels of penetration of an agent or composition can be determined by techniques known to those of skill in the art. For example, radiolabeling of the active compound, followed by measurement of the amount of radiolabeled compound absorbed by the skin enables one of skill in the art to determine levels of the composition absorbed using any of several methods of determining skin penetration of the test compound. Publications relating to skin penetration studies include Reinfenrath, W G and G S Hawkins. The Weaning Yorkshire Pig as an Animal Model for Measuring Percutaneous Penetration. In: Swine in Biomedical Research (M. E. Tumbleson, Ed.) Plenum, New York, 1986, and Hawkins, G. S. Methodology for the Execution of In Vitro Skin Penetration Determinations. In: Methods for Skin Absorption, B W Kemppainen and W G Reifenrath, Eds., CRC Press, Boca Raton, 1990, pp. 67-80; and W. G. Reifenrath, Cosmetics & Toiletries, 110:3-9 (1995).

For some applications, a long acting form of agent or composition may be administered using formulations known in the arts, such as polymers. The agent can be incorporated into a dermal patch (Junginger, H. E., in Acta Pharmaceutica Nordica 4:117 (1992); Thacharodi et al, in Biomaterials 16:145-148 (1995); Niedner R., in Hautarzt 39:761-766 (1988)) or a bandage according to methods known in the arts, to increase the efficiency of delivery of the drug to the areas to be treated.

Optionally, the topical formulations described here can have additional excipients for example; preservatives such as methylparaben, benzyl alcohol, sorbic acid or quaternary ammonium compound; stabilizers such as EDTA, antioxidants such as butylated hydroxytoluene or butylated hydroxanisole, and buffers such as citrate and phosphate.

Parenteral Administration

The active compound may also be administered parenterally or intraperitoneally. Dispersions can also be prepared in glycerol, liquid polyethylene glycols, and mixtures thereof and in oils. In some embodiments, the dispersions may be prepared in 30% Capsitol (CyDex, Inc., Lenexa, Kans., USA). Capsitol is a polyanionic β-cyclodextrin derivative with a sodium sulfonate salt separated from the lipophilic cavity by a butyl ether spacer group, or sulfobutylether (SBE). The cyclodextrin may be SBE7-β-CD.

Adjuvants

The composition may be administered in an adjuvant, co-administered with enzyme inhibitors or in liposomes. Adjuvant is used in its broadest sense and includes any immune stimulating compound such as interferon. Adjuvants contemplated herein include resorcinols, non-ionic surfactants such as polyoxyethylene oleyl ether and n-hexadecyl polyethylene ether. Enzyme inhibitors include pancreatic trypsin. Liposomes include water-in-oil-in-water CGF emulsions as well as conventional liposomes.

Prevention of Microorganism Growth

Under ordinary conditions of storage and use, these preparations may contain a preservative to prevent the growth of microorganisms.

The prevention of the action of microorganisms can be brought about by various antibacterial and antifungal agents, for example, parabens, chlorobutanol, phenol, sorbic acid, thirmerosal, and the like. In many cases, it is possible to include isotonic agents, for example, sugars or sodium chloride. Prolonged absorption of the injectable compositions can be brought about by the use in the compositions of agents delaying absorption, for example, aluminium monostearate and gelatin.

Sterile injectable solutions are prepared by incorporating the active compound in the required amount in the appropriate solvent with various of the other ingredients enumerated above, as required, followed by filtered sterilisation. Generally, dispersions are prepared by incorporating the sterilised active ingredient into a sterile vehicle which contains the basic dispersion medium and the required other ingredients from those enumerated above. In the case of sterile powders for the preparation of sterile injectable solutions, the methods of preparation may include vacuum drying and the freeze-drying technique which yield a powder of the active ingredient plus any additional desired ingredient from previously sterile-filtered solution thereof.

Pharmaceutically Acceptable Carrier

As used herein “pharmaceutically acceptable carrier and/or diluent” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, use thereof in the therapeutic compositions is contemplated. Supplementary active ingredients can also be incorporated into the compositions.

Dosage Unit Forms

Pharmaceutical compositions may be formulated in dosage unit form for ease of administration and uniformity of dosage.

Dosage unit form as used herein refers to physically discrete units suited as unitary dosages for the subjects to be treated; each unit containing a predetermined quantity of active material calculated to produce the desired therapeutic effect in association with the required pharmaceutical carrier. The specification for the novel dosage unit forms are dictated by and directly dependent on (a) the unique characteristics of the active material and the particular therapeutic effect to be achieved, and (b) the limitations inherent in the art of compounding such as active material for the treatment of disease in living subjects having a diseased condition in which bodily health is impaired.

The principal active ingredients are compounded for convenient and effective administration in effective amounts with a suitable pharmaceutically acceptable carrier in dosage unit form. In the case of compositions containing supplementary active ingredients, the dosages are determined by reference to the usual dose and manner of administration of the ingredients.

### EXAMPLES

The mammalian placenta is a critical source of factors responsible for remodelling of the maternal cardiovascular system to accommodate the growing fetus.

ELABELA (ELA), a recently discovered alternate ligand for APLNR, is secreted by placental trophoblasts and safeguards pregnancy bidirectionally, ensuring proper cardiovascular development in the fetus, and regulating the maternal endothelium to prevent gestational hypertension.

Ela knockout pregnant mice display elevated blood pressure, proteinuria, which can be reversed by infusion of synthetic ELA throughout pregnancy, and Ela null fetuses have major cardiovascular defects.

ELA levels are paradoxically elevated in women diagnosed with preeclampsia, which might reflect a compensatory effort to ameliorate pre-eclampsia symptoms, or a dysfunctional ELA pathway.

Surprisingly, these phenotypes are not recapitulated in Apelin or Aplnr null mice, while Apelin; Ela double nulls are rescued from pre-eclampsia symptoms and placental defects.

We propose that ELA is able to fine tune Aplnr activity and therefore could be a therapeutic candidate for pregnant women suffering from pre-eclampsia.

### Example 1. Deletion of Ela Causes Mid-Gestation Death Due to Cardiovascular Defects

Example 1 is shown in FIG. 1. FIG. 1 shows that deletion of Ela causes mid-gestation death due to cardiovascular defects.

### Example 2. Ela is a Placental Hormone Required for Placental Vasculogenesis

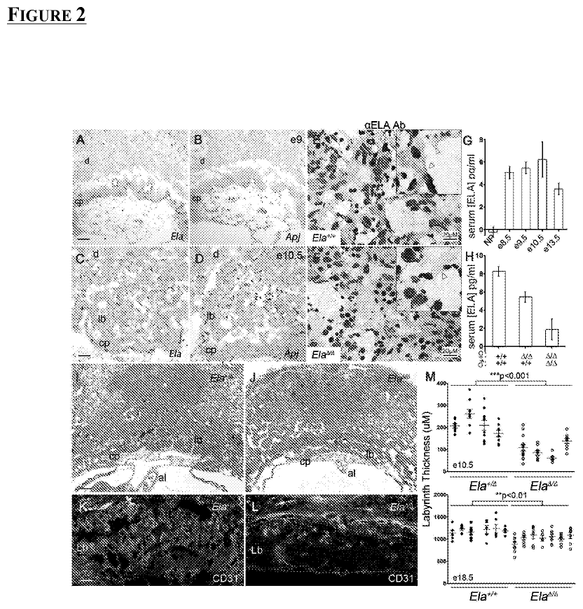

Example 2 is shown in FIG. 2. FIG. 2 shows that Ela is a placental hormone required for placental vasculogenesis.

### Example 3. Loss of Ela During Pregnancy Predisposes to Pre-Eclampsia

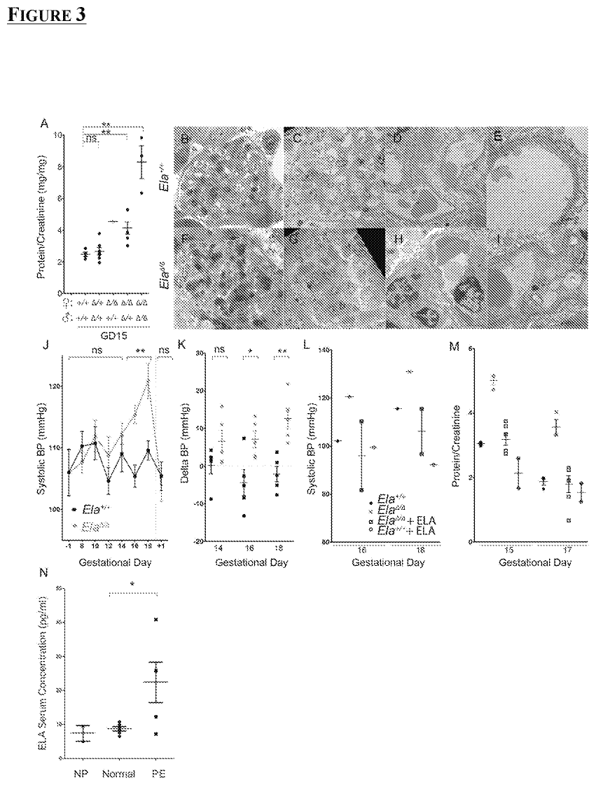

Example 3 is shown in FIG. 3. FIG. 3 shows that loss of Ela during pregnancy predisposes to pre-eclampsia.

### Example 4. Deletion of Ela and Apj have Opposite Effects on Proteinuria and Gestational Blood Pressure

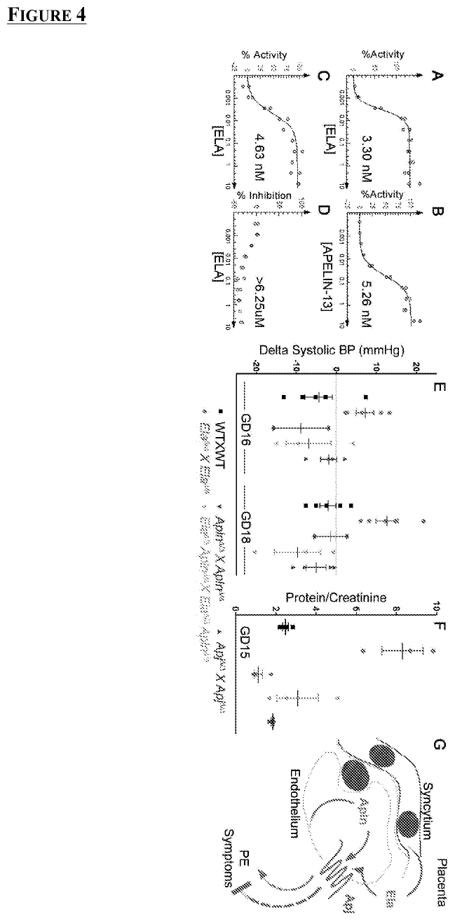

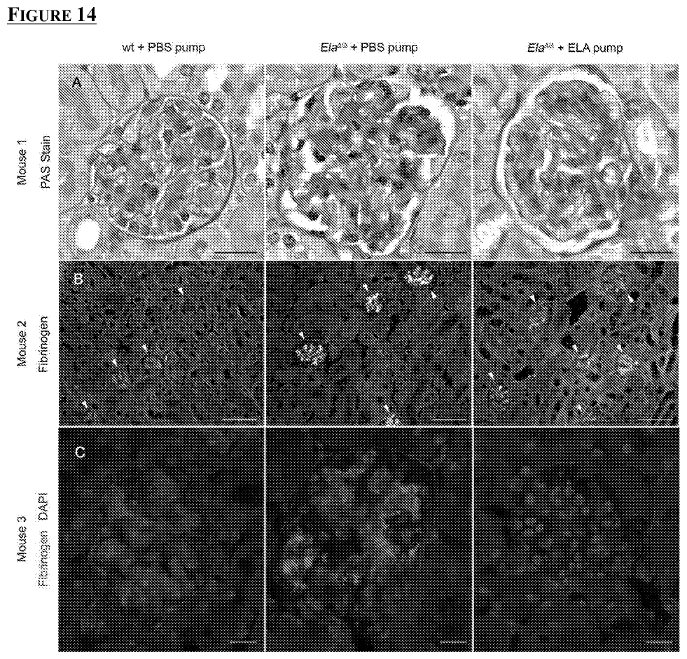

Example 4 is shown in FIG. 4. FIG. 14 shows that deletion of Ela and Apj have opposite effects on proteinuria and gestational blood pressure.

### Example 5. Materials and Methods: Mice

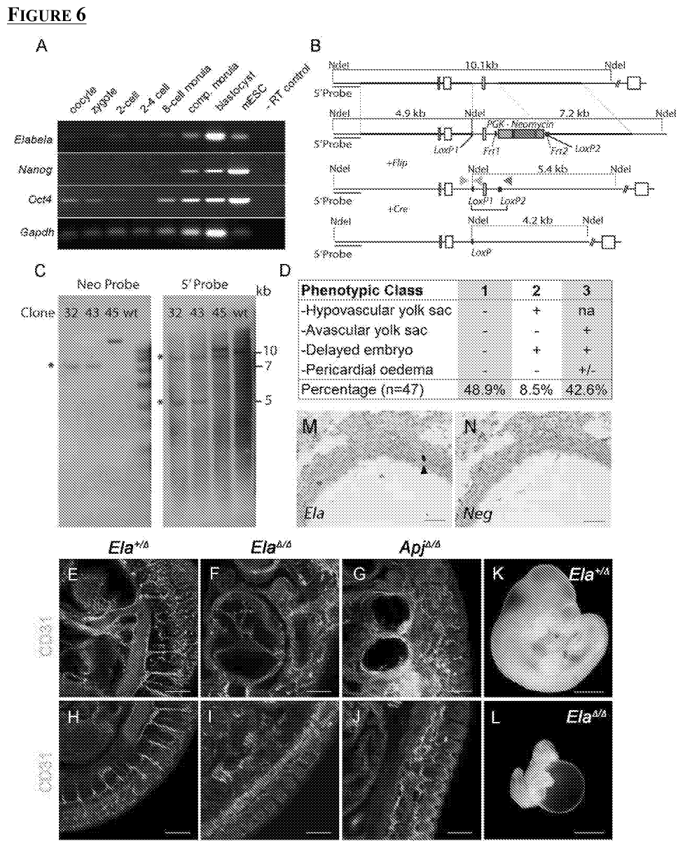

Exon 3 of mouse Elabela (NC_000074.6 and NM_001297554.1) encoding the mature peptide was flanked with loxp sites by homologous recombination-mediated insertion of a targeting vector in C57B6/J mouse embryonic stem cells (mESCs). The targeting vector consists loxp sites flanking exon 3, PGK-neomycin (neo) cassette flanked by frt sites in between exon 2 and 3′ loxp site, and 5′ and 3′ homology arms. Electroporated mESCs was subjected to G418 selection, and G418-resistant clones were pre-screened by PCR for presence of neo cassette. We selected 3 neo-positive clones for southern blotting with a neo probe and 5′probe following NdeI digestion of genomic DNA. Clones 32 and 43 yielded bands of expected sizes with both probes (FIG. 6B), while clone 45 represented a random integration. Clones 32 and 43 were injected into B6(Cg)-Tyr^{c-2/J} congenic B6 albino recipient blastocysts (Jackson Laboratory). We achieved germline transmission with one F₀ chimera from clone 32, which gave rise to black-coated F₁ offspring. These F₁ mice were bred with a CMV-flp recombinase transgenic mouse (C57BL/6-Tg(CAG-Flpe)2Arte, Taconic) to remove the neo cassette to generate the Ea^{flox} allele. Ea^{flox/wt} heterozygotes were bred with Zp3-cre (C57BL/6-Tg(Zp3-cre)1Gwh/J, Jackson Laboratory) recombinase transgenic mouse to generate the [symbol] knockout allele. [symbol] heterozygotes were backcrossed with C57B6/J for >8 generations before inter-crossing to generate [symbol] homozygotes. To avoid possible trans-generational adaptation to the loss of Ela, the [symbol] allele was maintained in the heterozygous state unless otherwise stated. All mouse procedures are done in compliance with IACUC protocols #080361, 110673 and 140907 with the approval of the Biological Resource Center, A*STAR and Agri-Food & Veterinary Authority of Singapore.

### Example 6. Materials and Methods: LacZ Staining

Apln knockout animals were a gift from Quertermous and colleagues (Ref A15). Apln is encoded by the X chromosome and the targeted Apln null allele (Apln^{LacZ}) contains a β-galactosidase transgene transcribed under the control of the endogenous Apln promoter. [symbol] ; Apln^{+/+} females were mated to [symbol] ; Apln^{LacZ/Y} and female embryos of genotype [symbol] ; Apln^{LacZ/+} were compared with female controls of genotype Ela^{+/+}; Apln^{LacZ/+} derived from mating between wt females and Ela^{+/+}; Apln^{LacZ/Y} males. Wholemount LacZ staining of [symbol] ; Apln^{LacZ/+} and Ela^{+/+}; Apln^{LacZ/+} embryos and placentas were performed in parallel on e9.5 litters harvested at the same time according to the protocol described by Barker and colleagues (Ref A31). Briefly, embryos and placentas were fixed in freshly prepared glutaraldehyde fixative for 1 hour at RT, washed 5× with freshly prepared washing buffer, and incubated with ultrapure X-gal substrate (Invitrogen 15520034) overnight at 30° C. in the dark. Staining was stopped by washing and fixing in 4% paraformaldehyde. A stereomicroscope (Leica; M205 FA) was used to capture images of wholemount placentas in successive focal planes. Images were then manually merged in the Z-dimension with Photoshop CS5 (Adobe).

### Example 7. Materials and Methods: In Situ Hybridization (WISH and RNAScope)

Wholemount in situ hybridization (WISH) was performed according to protocols available on our lab's website (https://sites.google.com/a/reversade.com/www/protocols/). RNAScope on mouse embryos, mouse tissues and human chorionic villi was performed with Advanced Cell Diagnostics' HD2.0 and HD2.5 brown kits according to the manufacturer's recommendations, using custom mEla and hELA (Apela Cat #416811 and APELA Cat #427251) C2 probes. 1^{st} and 3rd trimester human placental tissues used for IHC and RNAScope were collected through the Preeclampsia And Non-preeclampsia DAtabase (PANDA), an obstetrical biosample effort approved by the institutional review board of the Academic Medical Center, University of Amsterdam. Informed consent was obtained from all women. All RNAScope tissues were fixed in neutral buffered formalin (NBF) (16-32 hours) prior to paraffin embedding and sectioning.

### Example 8. Materials and Methods: Immunohistochemistry (IHC) and Immunofluorescence (IF)

Wholemount antibody staining against CD31 (MEC13.3, BioLegend) was performed as described by (Ref A32, A33). Briefly, for wholemount IF, e10.5 embryos were fixed in 4% (wt/vol) PFA in PBS, overnight at 4° C., dehydrated through a methanol series on ice, bleached with 5% hydrogen peroxide in 4:1 Methanol:DMSO for 4 hours at room temperature, blocked with 3% BSA (Sigma A7030) in PBS-MT (3% (wt/vol) skim milk and 0.1% (vol/vol) Triton X-100) solution on ice for 1 h. MEC13.3 was added at 1:100 for 2 days, rocking at 4° C. Embryos were washed with PBS-MT 5× (1 hour each at 4° C.), stained with secondary antibody (AF-488, Molecular Probes) at 1:500 overnight at 4° C., washed 3× with PBS-T (0.5% (vol/vol) Triton X-100), and dehydrated to 100% methanol. They were mounted onto coverslips in low melting agarose, incubated in methanol to dehydrate the agarose, cleared in BABB solution (1 part benzyl alcohol: 2 parts benzyl benzoate, Sigma) and mounted onto Fastwells for confocal imaging (FV1000, Olympus). Images presented are maximally projected along the z-axis. A similar procedure applies for wholemount IHC, except using anti-rat HRP (Jackson Laboratories, 1:2000) as the secondary antibody, and 3,3′-Diaminobenzidine Peroxidase Substrate (SIGMAFAST DAB with Metal Enhancer, Sigma D0426). After extensive washes in stop buffer (5 mM EDTA in PBS), embryos were then dehydrated through a methanol series and cleared using BABB, and imaged with a stereomicroscope (Leica; M205 FA).

For section-based staining, two methods of tissue preparation were used: formalin-fixed paraffin-embedded sections (FFPE) and fresh frozen cryosections (FF). e9.5-10.5 embryos were fixed or directly frozen within the decidua while e18.5 placentas were removed from concepti and fixed separately. Standard procedures for IHC and IF were used, using 5% normal goat serum as a blocking agent, unless staining for phospho-proteins, where 3% BSA was used instead. The following proteins were detected using FFPE-IHC: ELABELA (α C ELA Ab); Syncytin-1 (MyBioSource, MBS2516746), phosphorylated-histone-H3 (Ser10) (Millipore 06-570); using FF-IHC: Hif1α (Abcam ab114977), ELA (custom α ELA C pAb), Alkaline phosphatase (MyBioSource, MBS2524098), cleaved Caspase 3 (Cell Signaling Technologies 5A1E); phosphorylated-histone-H3 (Ser10); using FF-IF: Hydroxy-HIF-1α (Pro564) (Cell Signaling Technologies D43B5). Sections were imaged with an inverted upright microscope (Zeiss Axiolmager).

Fibrinogen IHC on cryosections was performed as described (Ref A34) using rabbit α-Fibrinogen whole antiserum (Abcam ab34269). PAS staining was performed on rehydrated FFPE sections by soaking for 15 minutes in Schiff reagent followed by counterstaining in Mayer's hematoxylin.

### Example 9. Materials and Methods: Western Blotting

Individually dissected e10.5 yolk sacs were lysed with PhosphoSafe extraction reagent (EMD Millipore, 71296) and subjected to SDS-PAGE. Antibodies used were against CD31 (Abeam ab28364), phosphorylated-histone-H3 (Ser10) (Millipore 06-570) and Actin (Clone C4, Millipore).

### Example 10. Materials and Methods: Mouse Biometric Measurements

Pregnant females of the indicated genotype were placed on gestational day (GD) 15 into individual metabolic cages (Techniplast Metabolic Mice) with adlib food and water from 10 am-10 pm. Urine samples were collected (directly into a 1.5 ml Eppendorf tube stuck to the lip of the collecting funnel of metabolic cage) and protein/creatinine ratio was measured using BioAssay's DPCR kit (DPCR-100) according to manufacturer's instructions. Each sample was measured at two different dilutions, each in duplicate, and the average reading was used.

Systolic blood pressure was measured using the CODA 4-channel tail cuff platform with small occlusion and VPR cuffs and medium mouse holder (Kent Scientific). Mice were pre-conditioned by measuring baseline blood pressure on 3 separate days at the same time of the day (2 pm) before they were time-mated to males. Each plugged mouse was then measured on GD 8, 10, 12, 14, 18 and 1 day post-parturition (typically 20 days post coitum). As much as possible, mice in all experimental groups were measured together on the same day. But this was not always possible as in a large cohort, they were not all plugged on the same day. However, great measures were taken to ensure that mice were exposed to the same environmental conditions (room, handler, noise levels, temperature, lighting, etc) and were measured at the same time each day (14:00 o'clock) to minimise inter-individual variations. All measurements for each mouse, from pre-training to post-parturition, were done in the same channel with the same cuffs to minimize inter-channel and cuff variation. Mice were kept immobilized in a holder on a heated platform (level 3) 15 minutes prior to commencement and throughout the measurement session, with tail skin temperature reaching and maintaining at 30° C. (as measured by infrared thermometer) during measurements. Each day's BP measurements were performed using the following parameters: 5 pre-conditioning cycles, 20 regular cycles, 5 seconds between each cycle, maximum cuff pressure of 150 mmHg. An average of all accepted cycles after removal of outliers was taken as the day's systolic BP. Delta systolic blood pressure=systolic BP_{GDx.}−Systolic BP_{Non-Pregnant}.

### Example 11. Materials and Methods: Osmotic Pumps

ELA peptide was synthetically produced by GL Biochem to reach >98% purity. Upon receipt, purity of peptide was checked by HPLC and mass spectrometry to ensure the correct mass of 3964.85. Lyophilized peptide was reconstituted with PBS, dialyzed with Slide-A-Lyzer dialysis cassettes (MW2500 cutoff, Thermo Fischer) to remove any small contaminants. Dialyzed peptide was relyophilized and stored at −80° C. until ready for use. Purified peptide was then resuspended to 2.5 mg/ml in water and infused at 0.5 mg/kg/day using subcutaneous Alzet pumps (model 1002) which delivered the peptide at a rate of 0.25 μl/hour. The prepared pumps were incubated overnight at 37° C. for equilibration before subcutaneous implantation on the back via a shoulder incision into pregnant female mice on GD7. A minimum of 7 days followed before mice were subjected to BP measurements to allow adequate wound healing. Exogenous ELA was detected in maternal serum via ELISA (at 2-3 fold that of endogenous levels), confirming that it entered systemic circulation.

### Example 12. Materials and Methods: Placental Transcriptomic Analysis

E9.5 placentas were dissected from wt, [symbol] and [symbol] embryos derived from wt×wt; [symbol] × [symbol] and [symbol] ×Apln^{Δ/Y} crosses respectively. [symbol] , [symbol] and wt mothers were littermates. Maternal decidual tissue was removed and the embryonic placenta was immediately snap-frozen. The corresponding embryos were fixed and assessed for vascular development and somatic stage after staining for CD31, and only the pair of wt and [symbol] placentas where wt and [symbol] class 1 embryos were developmentally matched was used for RNASeq. RNA was extracted using RNAeasy (Qiagen) and had to exceed a RIN value of 9 on the BioAnalyzer (Agilent) for further consideration.

### Example 13. Materials and Methods: Library Preparation (Stranded mRNA-Seq)

The total RNA quality and RIN value was confirmed with a Tapestation from Agilent and Agilent RNA ScreenTape. A high RIN value of 8 and above indicates that the total RNA quality was good and suitable for library preparation. Library preparation was done using a commercially available kit, Illumina® TruSeq Stranded mRNA LT Sample Prep Kit, following the manufacturer's protocol. During the library construction, the enriched mRNA was fragmented to obtain inserts with fragment size of 120-200 bp (a median size of 150 bp). The quality of the library was checked using Agilent D1000 ScreenTape. A single peak in the expected region of ˜280 bp should be observed indicating that the library was good and suitable for sequencing.

### Example 14. Materials and Methods: Cluster Generation and Sequencing

The sample were linearized with 0.2N NaOH into single-stranded forms, they were then neutralized and diluted into 4 pM loading concentration with Hybridization buffer (HT1). The NEXTSEQ High Output was performed using the Illumina NEXTSEQ 500 Sequencers with the Illumina® Reagent v2 (75 cycle kit) Kit. The DNA were attached to the flowcell surfaces and amplified to clusters and attached with the Sequencing primers and run at 1×76 cycles, generating Single-Read 75 base-pair reads. The images were captured by the NextSeq Control Software (MCS), and the Real Time Analysis (RTA v2) software converted the images into Basecall (bcl) files. All the bcl files were then transferred to the server for storage and primary analysis.

### Example 15. Materials and Methods: RNA-Seq Data Analysis

In the primary analysis, the bcl files were converted into fastq files using the bcl2fastq. After the conversion, the fastq reads were filtered to remove all the reads that did not pass filtering, leaving only useable Passed Filtered (PF) reads. The primary analysis result was then generated as the Demultiplexed_Stats file and reviewed, and the PF fastq files were then passed on for further analysis. Reads from each library were mapped uniquely and independently using TopHat (version 2.0.11) against the mouse reference genome (mm9). The mapped reads were further analyzed using the Cufflinks program (version 2.2.1), and the expression levels for each transcript were quantified as fragments per kilobase of transcript per million mapped reads (FPKM) using mouse gencode.vM1 annotation.

Differential FKPM gene expression was determined using CuffDiff (cuffdiff v2.2.1 (4237)) with the following parameters: geometric library normalization, pooled dispersion estimation, 0.05 False Discovery Rate, minimum alignment count of 10, bias correction using mm9 reference sequence, and with cufflinks effective length correction. Genes where there was no expression above FPKM of 0.5 in any condition was excluded from further analysis. Gene set enrichment analysis was performed as described by (Ref A7). Normalized FPKM counts (after removal of “non-expressed” genes) for each sample were used as input GCT data for testing against gene sets in Molecular Signature Database using a similar workflow as that for conventional microarray data. We also generated a custom-made “tip cell” gene set derived from del Toro et al. (Ref A9), who used transcriptomic analysis of retinal endothelial cells isolated from Dll4^{+/-} mouse retinas (which have a massive overgrowth of tip cells compared to wt retinas) to define a set of tip cell enriched genes. Ten of the topmost upregulated and downregulated genes were validated by qPCR from the same samples to ensure correspondence with RNASeq data. Raw data are deposited in NCBI's SRA under GSM 2675549 (provisional). Below is the sequence of oligos used for qper:

| Gene | Primer Sequence |
|---|---|
| Esm1 | 5′ ggaggatgattttggtgacg 3′; |
| | 5′ ctggaagaagggaaagtcca 3′ |
| Apln | 5′ ccgagttgcagcatgaatc 3′; |
| | 5′ gcaacatcagtggcactcc 3′ |
| Adm | 5′ acaacgcacccctttatcag 3′; |
| | 5′ cggaactgcgaaggagtatc 3′ |
| Plgf | 5′ tgaaggcatgtagaggggac 3′; |
| | 5′ cactctgcctgtgttccaga 3′ |
| Vegfa | 5′ gaccctggctttactgctgt 3′; |
| | 5′ caccagggtctcaatcggac 3′ |
| Igfbp5 | 5′ tttgcctcaacgaaaagagc 3′; |
| | 5′ gtaggtctcttcagccatctcg 3′ |
| Igfbp3 | 5′ ggagcagtacccgctgag 3′; |
| | 5′ attgtgctcctcctcggac 3′ |
| Flt4 | 5′ gtggctctgcctcggact 3′; |
| | 5′ gctgtcccctgcaggatatg 3′ |
| sFlt1 | 5′ tgcttcatagcagcaacctg 3′; |
| | 5′ actgtacgcatcctgtgctg 3′ |

### Example 16. Materials and Methods: Allantoic Explants

Allantoic explants were microdissected from 4-5 somite stage embryos on the morning of the 8^{th} day of conception in dissection medium (DMEM+Hepes+7.5% FCS), and cultured in DMEM+NaHCO₃+50% rat serum, on poly-L-lysine coated microwells (Ibidi). 1 hour after placing into culture, the following ligands were added: PBS, 2.5 μM APLN-36 (Sigma), 2.5 μM ELA or both ELA and APLN-36 together. 12 hours later, the explants were harvested by direct lysis and RNA extracted with the Nucleospin RNA XS kit (Machery Nagel). cDNA was made using the SMARTer® Pico PCR cDNA Synthesis Kit (Clontech) and gene quantitation was performed using digital droplet PCR (Biorad) with EvaGreen and PrimeTime probes listed below:

| Gene | Biorad ddPCR Probe number |
|---|---|
| Apln (ddPCR) | dMmuEG5077462 |
| Foxoa3 (ddPCR) | dMmuEG5062299 |
| Hif3-alpha (ddPCR) | dMmuEG5079239 |
| Esm1 (ddPCR) | dMmuEG5075252 |
| Actin (ddPCR) | dMmuEG5193531 |

### Example 17. Materials and Methods: Placental Esm1 Staining

Since Esm1-positive cells are very rare in the placenta, we detected them via confocal imaging of whole-mount immunofluorescently labelled thick sections. E9.5 placentas were dissected and fixed for 15 minutes at room temperature in 4% PFA. They were washed and embedded into 2.5% low melting temperature agarose and sectioned on a vibratome (Leica) at 200 μm (Velocity 6, speed 7). Slices were further fixed for 5 minutes at room temperature, and blocked for 1 hour in 1% BSA/0.3% TX-100 in PBS at room temperature, followed by staining with anti-CD31 (MEC13.3, BioLegend) and anti-Esm1 (R&D System, AF1999) overnight at 4° C. Sections were washed 3× (10 minutes each) with 1% BSA/0.3% TX-100, stained with chicken anti-rat-AF488 (Molecular probes 1:1000) and donkey anti-goat-647 (Molecular probes, 1:500) for 1.5 hours at room temperature. After 3 washes, sections were dehydrated to 100% methanol, and cleared in BABB before mounting onto slides in BABB. The median section of each placenta was used for imaging, and the labyrinth area under the maternal central canal was chosen. Confocal imaging was performed on an Olympus FV1000, with identical settings for all samples acquired. 77 z-stacks were acquired per section and the number of Esm1-positive cells per section was deduced using the 3D object counter plugin in FIJI (Ref A35, Ref A36).

### Example 18. Materials and Methods: Transmission Electron Microscopy

At GD18.5, kidneys were harvested from wt and [symbol] mothers mated to wt and [symbol] ° males respectively. They were manually dissected into 2 mm thick pieces containing cortical regions in washing buffer and fixed in 0.1 M phosphate buffered glutaraldehyde (2%) overnight at 4° C. 20 μm thick sections were cut using the vibratome and sections were washed in 0.1 M cacodylate buffer and post-fixed in 1% OsO4 containing 1.5% K₃Fe(Cn₆) for 2 hours, dehydrated and embedded in epoxy resin. Sections were collected on copper grids, counterstained and images were acquired on a JEM-1010. 5 images per kidney sample per magnification were acquired, and representative images are shown.

### Example 19. Materials and Methods: ELISAs

ELA: Serum from female mice was harvested at the indicated gestation day by cardiac puncture followed by immediate centrifugation to remove haematocrit. The fibrin clot was squeezed and removed by further centrifugation and the serum was immediately snap-frozen and stored at −80° C. until all samples were ready for measurement at the same time. The custom ELA ELISA was performed as described previously (Ref A1) with the following modifications: the detection rabbit polyclonal α C antibody was biotinylated using One-Step Antibody Biotinylation kit (Miltenyi Biotec) and used at 0.5 ug/ml. Streptavidin-HRP was used as an amplifying secondary antibody. ELA peptide, used as a standard, was diluted in serum from non-pregnant NSG female mice which we found to give a low level of background signal.

Plgf, Vegf, Apln, sFlt1: Blood from female mice at GD 15 and 18 was harvested from the lateral saphenous vein using Microvette CB K2E 300 μl tubes (Starstedt), and immediately spun down. Plasma supernatant was collected and snap frozen until all samples were ready for measurement. ELISA kits for mouse Plgf, Vegf, Apln and sFlt1 were purchased from Ray Biotech and performed according to the manufacturer's instructions. sFlt1 ELISA kit was additionally validated using an independently obtained sFlt1 recombinant standard, and by the observation that circulating sFlt1 levels increased throughout gestation as is expected.

### Example 20. Materials and Methods: Invasion Assay

JAR choriocarcinoma cells were serum-starved for 24 hours after which 50,000 cells were plated on 100 μl 10× diluted Matrigel coated 8.0 μm cell culture inserts in the presence of different concentrations of ELA recombinant peptide. Invasion took place for 27 hours after which the membranes were fixed, covered in Vectashield with DAPI and coverslipped after which the underside of the membrane was counted. Counting was performed by taking pictures of 10 random fields per membrane after which ImageJ was used to count the number of cells.

### Example 21. Materials and Methods: Statistics

All data are presented as mean±standard error of mean (s.e.m.). Statistical approaches to test differences of means were performed using Prism 5.0. The type of test used in each panel is indicated in the accompanying figure legend. These include two sample/paired Student's t-test, 1 and 2-way ANOVA with two-sided testing, unless otherwise indicated. Where p values are not explicitly indicated, significance levels follow the convention of *p<0.05, **p<0.01, ***p<0.001. N numbers are explicitly indicated for bar graphs. In all panels, data points in scatter plots represent individual biological replicates.

### Example 22. Zygotic Deletion of Ela Causes Mid-Gestation Lethality Due to Cardiovascular Defects and Phenocopies Loss of Apj

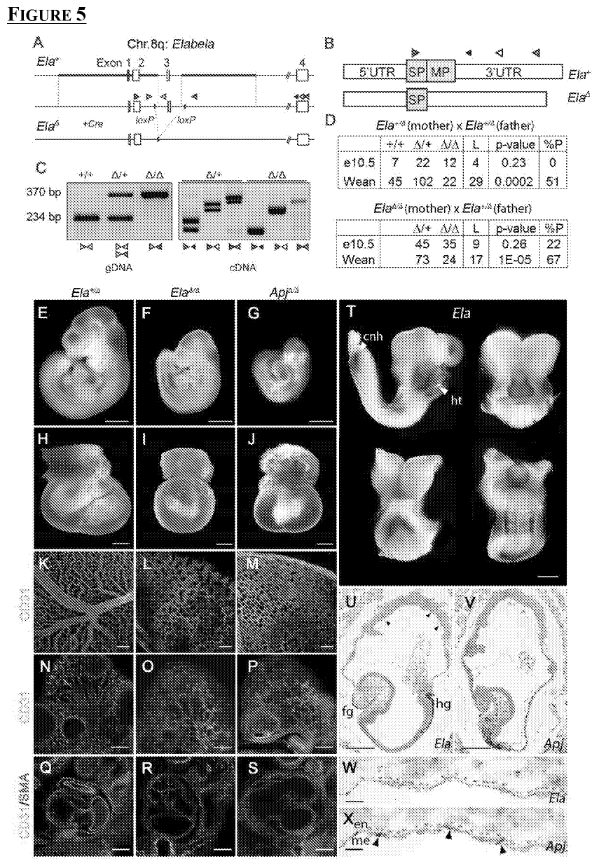

In order to delineate the contribution of ELA to mammalian development, we generated Ela knockout ( [symbol] ) mice using homologous recombination to delete exon 3 encoding the mature ELA peptide (FIG. 5A, FIG. 5B and FIG. 6B and FIG. 6C).

This strategy did not result in nonsense-mediated decay of the Ela^{Δ} mRNA (FIG. 5C), and presumably preserves the potential non-coding functions of the Ela transcript (5). Only half of the expected [symbol] mice from heterozygous intercrosses were obtained at weaning (FIG. 5D) (p<0.001, Chi-squared test with df=1).

Notably, this reduced recessive Mendelian inheritance was even more pronounced for [symbol] embryos carried by [symbol] mothers (67%) than by Ela^{Δ/+} mothers (51%) (FIG. 5D). This apparent maternal contribution is not due to Ela mRNA being deposited in the oocyte, since the onset of Ela transcription is strictly zygotic (FIG. 6A). Rather we surmise that ELA might be provided by the maternal circulation or uterine environment.

At e10.5, [symbol] embryos can be grouped into 3 classes: 48.9% were phenotypically normal (Class 1), 8.5% were delayed with a hypovascular yolk sac (Class 2) and 42.6% had avascular yolk sacs and severe embryonic vascular malformations (Class 3, FIG. 6D) that are similar to those previously reported for Apj knockouts (FIG. 5E to FIG. 5J). In affected [symbol] embryos, vasculogenesis appears to initiate, as evidenced by the presence of a CD31/Pecam⁺ endothelial plexus, which subsequently fails to undergo remodeling and angiogenic sprouting to form organized vitelline vessels, dorsal aorta, outflow tract and intersomitic vessels (FIG. 5K to FIG. 5S, FIG. 6E to FIG. 6J).

The heart tube is poorly looped with reduced smooth actin muscle (SMA) staining (FIG. 5Q to FIG. 5S), and the most severely affected embryos (Class 3) have pericardial edema (FIG. 6K and FIG. 6L).

These cardiac defects are consistent with the first post-gastrulation expression of Ela in the primitive foregut overlying the developing heart tube (FIG. 5T to FIG. 5U) (6).

Surprisingly, Ela is not detected in endothelial precursors of the yolk sac (FIG. 5W), whereas Apj expression is ubiquitous in embryonic, allantoic and yolk sac mesoderm, which gives rise to endothelial cells (FIG. 5V, FIG. 5X). The expression patterns of Ela and Apj suggest that the observed cardiac defects are partly due to insufficient blood flow to stimulate angiogenesis.

### Example 23. Ela is a Pregnancy Hormone Required for Placental Angiogenesis

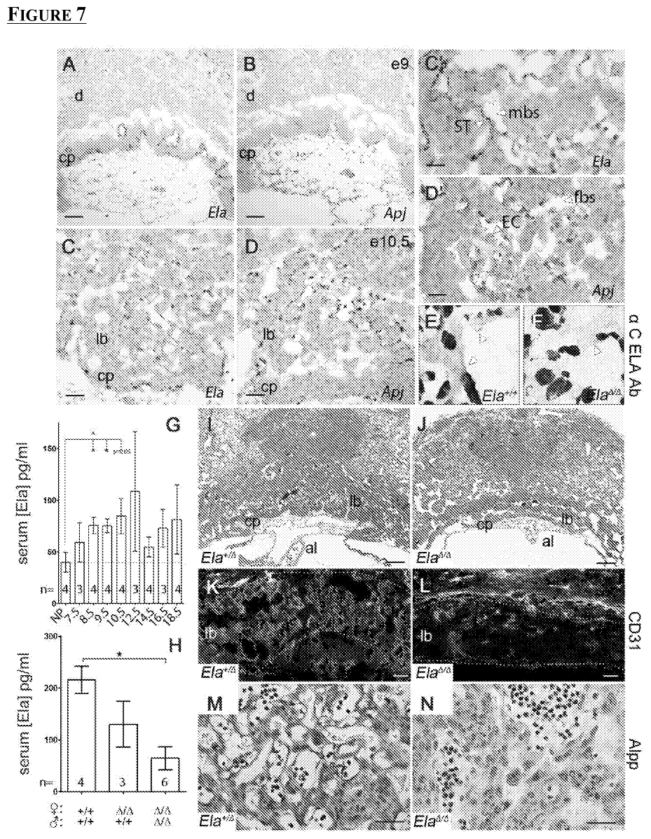

Outside of the developing heart tube, Ela is first detected in the chorionic trophoblast of the developing placenta (FIG. 5U, FIG. 6M and FIG. 6N) and is robustly up-regulated after allantoic fusion (FIG. 7A), becoming restricted to syncytiotrophoblasts (ST) at e10.5 (FIG. 7C and FIG. 7C′).

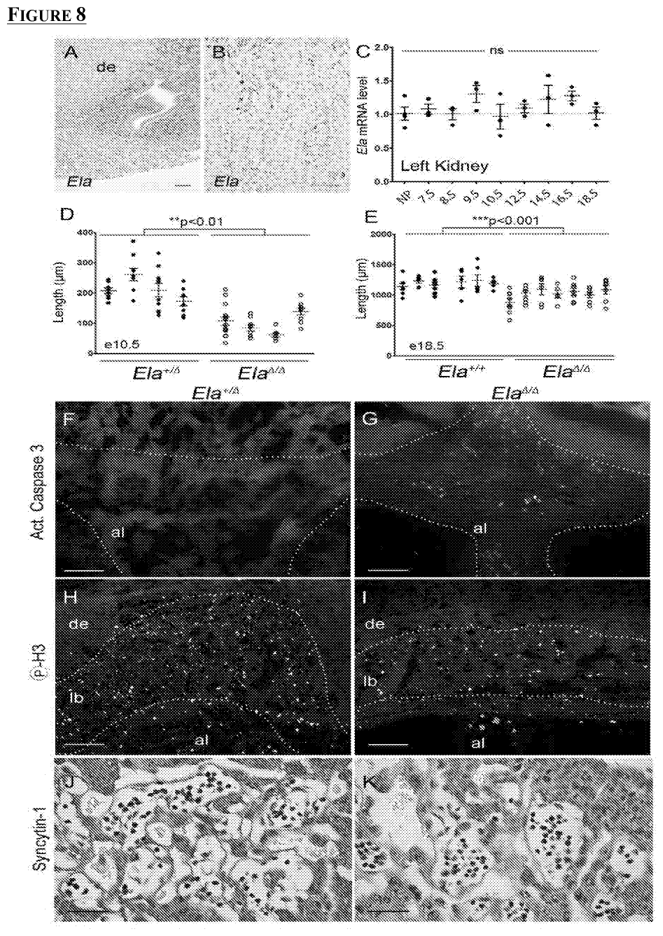

Accordingly, ELA protein is detected by immunohistochemistry in wild-type (wt) STs but not in [symbol] placentas (FIG. 7E and FIG. 7F). ELA-positive STs are juxtaposed to Apj-expressing fetal endothelial cells (FIG. 7B, FIG. 7D and FIG. 7D′). Hence, ELA may signal to Apj-expressing cells in a paracrine fashion, but may also be circulating systemically since the chorioallantoic placenta is perfused by maternal and fetal blood. Indeed, endogenous ELA is detected by ELISA in the serum of pregnant females, peaking at midgestation, but not in non-pregnant mice (FIG. 7G). Systemic ELA in a pregnant mother is contributed both maternally and embryonically (FIG. 7H), the former reflecting secretion from the maternal endometrial stroma and kidneys (FIG. 8A to FIG. 8C) and the latter from embryonically-derived STs (FIG. 7C). We therefore conclude that ELA is a pregnancy-associated hormone secreted by the developing conceptus and placenta.

[symbol] placentas from affected embryos have thin labyrinths (FIG. 7I and FIG. 7J, FIG. 8D and FIG. 7E) with poor vascularization (FIG. 7K and FIG. 7L), increased apoptosis (FIG. 8F and FIG. 8G) and reduced proliferation (FIG. 8H and FIG. 8I). [symbol] placentas from unaffected (Class 1) or mildly affected (Class 2) embryos, which are intermediately vascularized, nonetheless exhibit delayed ST differentiation, as indicated by reduced alkaline phosphatase and Syncytin-1 staining at e10.5 (FIG. 7M and FIG. 7N, FIG. 8J and FIG. 8K). Although such placentas eventually develop allowing embryo survival, the labyrinth of mutant versus wt placentas remains thinner until the end of gestation (FIG. 8E).

### Example 24. Loss of Ela Causes Hypoxic Response and Upregulation of a Pro-Angiogenic Program

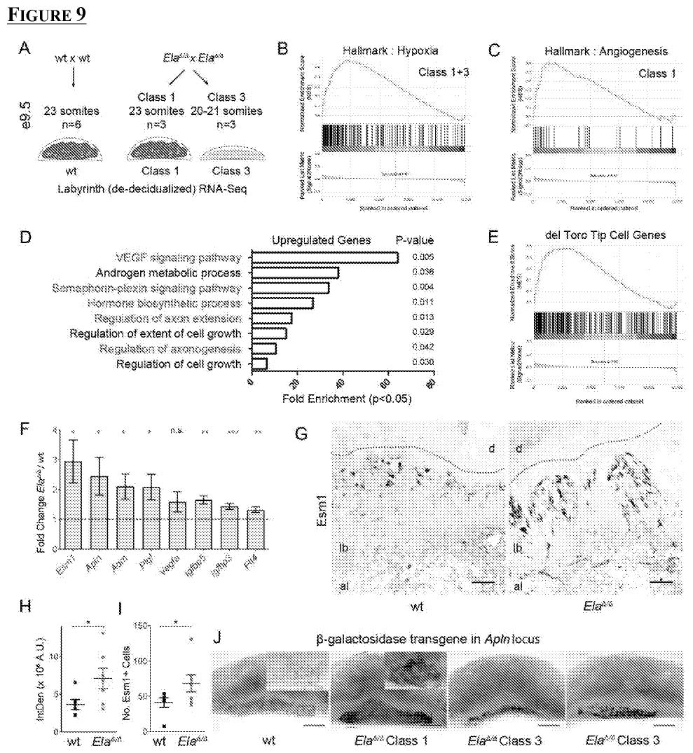

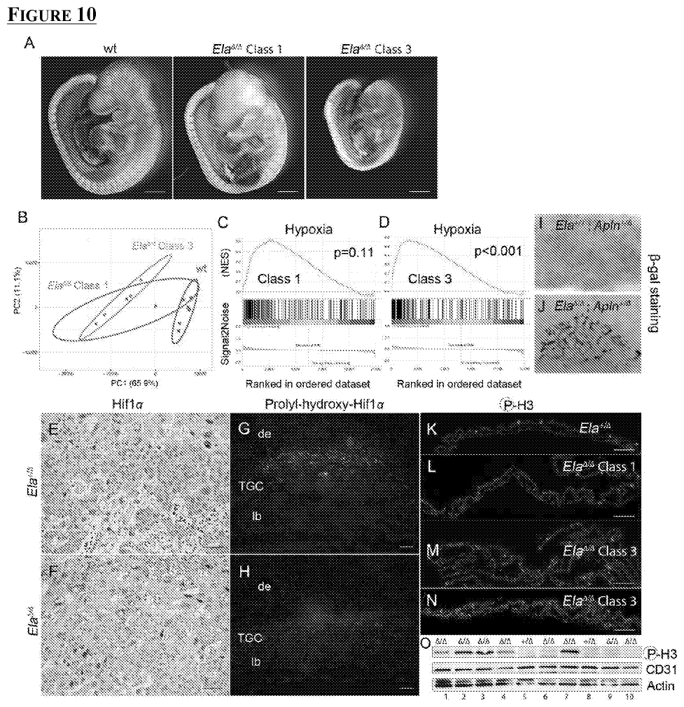

To understand the pathogenesis of Ela deficiency causing placental dysfunction, we isolated placentas denuded of maternal decidua from wt and [symbol] conceptuses (FIG. 9A). We chose to carry out the analysis by e9.5 to avoid the confounding transcriptional changes brought about by major cardiovascular anomalies seen at e10.5. [symbol] placentas were categorized into Class 1 or Class 3 based on the gross morphology of the corresponding embryos (FIG. 10A).

RNA sequencing and principal component analysis revealed that both Class 1 and 3 [symbol] placentas clustered closer to each other and away from wt placentas (FIG. 10B). Since Class 1 placentas are grossly indistinguishable from wt counterparts, these results indicate that the observed transcriptional changes are due to ELA deficiency rather morphological defects already present at the time of specimen collection.

Gene set enrichment analysis (GSEA) (7) revealed that Class 1 and 3 [symbol] placentas have a gene signature indicative of an elevated hypoxic response (FIG. 9B, FIG. 10C and FIG. 10D, Table E1, below).

TABLE E1
List of top 65 upregulated genes in [symbol] placentas

| Gene Accession | Gene Name | Ranked List Score |
|---|---|---|
| NM_008343 | Igfbp3 | 1.1986631 |
| NM_023612 | Esm1 | 1.1892145 |
| NM_016674 | Cldn1 | 1.1821203 |
| NM_010518 | Igfbp5 | 1.1229558 |
| NR_045668 | Gm5124 | 1.0909908 |
| NM_001109914 | Apold1 | 1.0872234 |
| NM_001190950 | Kcne3 | 1.0729139 |
| NM_133914 | Rasa4 | 1.0352502 |
| NM_001310544 | Samd4 | 1.0257573 |
| NM_001271705 | Pgf | 1.0125489 |
| NM_008029 | Flt4 | 1.0007496 |
| NM_008985 | Ptprn | 0.9983378 |
| NM_010097 | Sparcl1 | 0.9965701 |
| NM_153193 | Hsd3b2 | 0.9884683 |
| NR_110474 | Loc102636514 | 0.9834622 |
| NM_001081441 | Wdr86 | 0.9832099 |
| NM_177872 | Adamts3 | 0.9707761 |
| NM_001311074 | Flnc | 0.96913075 |
| NM_018881 | Fmo2 | 0.95004916 |
| NM_001001892 | H2-K1 | 0.94106656 |
| NM_008675 | Nbl1 | 0.93249136 |
| NM_016812 | Banp | 0.9275832 |
| NR_033578 | Gm15645 | 0.92513967 |
| NM_001159536 | Adcy3 | 0.9233715 |
| NM_008770 | Cldn11 | 0.92121845 |
| NM_008634 | Map1b | 0.91822815 |
| NM_009378 | Thbd | 0.9181567 |
| NM_178212 | Hist2h2aa2 | 0.9129521 |
| NM_019789 | Kcnip3 | 0.9050861 |
| NM_174850 | Micall2 | 0.9015534 |
| NR_028519 | Scarna6 | 0.9014383 |
| NR_051981 | H2-Q5 | 0.9000408 |
| NM_008689 | Nfkb2 | 0.89999384 |
| NR_030442 | Mir677 | 0.8992806 |
| NR_035445 | Mir1894 | 0.8988186 |
| NM_175547 | Nlrc3 | 0.8976624 |
| NM_138750 | Prom2 | 0.89608526 |
| NM_019800 | Acp7 | 0.8947844 |
| NR_015521 | 1700030c10rik | 0.89240986 |
| NM_001289699 | Prrt3 | 0.8872082 |
| NM_020518 | Vsig2 | 0.88462394 |
| NM_001017525 | Btbd11 | 0.8839835 |
| NM_008485 | Lamc2 | 0.8773124 |
| NM_173788 | Npr2 | 0.87634456 |
| NM_001033446 | Arl14epl | 0.87603563 |
| NM_009991 | Cyp17a1 | 0.8741302 |
| NM_001311076 | Lingo1 | 0.86763215 |
| NM_001048167 | Map6 | 0.8648775 |
| NM_144545 | Eif3j1 | 0.8640847 |
| NM_019450 | Il1f6 | 0.8635084 |
| NM_025383 | Nectin1 | 0.8634562 |
| NM_013912 | Apln | 0.86205393 |
| NM_008881 | Plxna1 | 0.8614464 |
| NM_001167996 | 1110032f04rik | 0.8613655 |
| NM_009627 | Adm | 0.86057186 |
| NM_018744 | Sema6a | 0.8604601 |
| NM_026955 | Vstm5 | 0.8579045 |
| NM_026730 | Gpihbp1 | 0.857619 |
| NM_010939 | Nrp2 | 0.8548263 |
| NM_028133 | Egln3 | 0.85419875 |
| NM_001083334 | Bin1 | 0.8490116 |
| NM_024279 | Spaca9 | 0.8467377 |
| NM_001130456 | Sema6b | 0.84638065 |
| NM_016741 | Scarb1 | 0.8443202 |
| NM_001287058 | Vegfa | 0.8408687 |

Consistent with this observation, [symbol] placentas have high levels of stabilized Hif1α (FIG. 10E and FIG. 10F), and decreased levels of prolyl-hydroxylated Hif1α (FIG. 10G and FIG. 10H) which is targeted for degradation under normoxia (8). Concurrently, and possibly as part of the elevated hypoxic response, Ela deficiency results in an up-regulation of pro-angiogenic genes, even in Class 1 placentas that are bereft of discernible vascular defects (FIG. 9C).

A close examination of differentially regulated genes revealed a dramatic enrichment in genes and pathways defining endothelial tip cells (FIG. 9D and FIG. 9E) (9). Tip cells form the leading edge of sprouting endothelial cells and migrate in response to pro-angiogenic signals (10). Functioning in the same way as axonal growth cones, tip cells extend filopodia to determine the direction of the angiogenic sprout, while trailing stalk cells proliferate to enable lumenogenesis and extension of the vascular sprout (11, 12).

Gene ontology analysis confirmed functional hallmarks of tip cell identity such as VEGF and Semaphorin signaling, hormone secretion, axonogenesis and filopodia extension (FIG. 9D). qPCR analysis validated the upregulation of tip cell markers and angiogenic genes including Vegfa, Apln, Plgf Esm1, Igfbp3, Flt4, Adm (FIG. 9F).

This was confirmed by direct immunostaining against endogenous Esm1, a specific tip cell marker (9, 13, 14), which was significantly up-regulated in both number and intensity in [symbol] placental sections, indicating ectopic tip cell differentiation (FIG. 9G to FIG. 9I). Using a Apln-LacZ knock-in reporter (15) as an alternate marker of tip cell identity, we found that there are indeed more Apln-positive tip cells in [symbol] labyrinths, with an overall stunted architecture characterized by little or no extension and branching of angiogenic sprouts (FIG. 9J, FIG. 10I and FIG. 10J).

This finding suggests that the absence of ELA causes an expansion of tip versus stalk cells, which is expected to impair lumenogenesis and sprout extension, such as in mice haplo-insufficient for the Notch ligand Dll4 (16, 17). Such an imbalance in tip cell identity is consistent with proliferative perturbations seen in [symbol] labyrinths, which are hypoproliferative (FIG. 8H and FIG. 8I), and [symbol] yolk sacs, which are conversely hyper-proliferative (FIG. 10K to FIG. 10O), with an overall negative impact on angiogenesis. Our results suggest that the ELA actively suppresses the expression of tip cell genes such as Esm1 and Apln.

### Example 25. Endogenous ELA Prevents Pre-Eclampsia (PE) and Exogenous ELA Administration Rescues PE Symptoms in Ela-Deficient Mice/ELA and APLN Have Different Biological Effects During Pregnancy/Administration of ELA to Pregnant Mice Rescues Endotheliosis

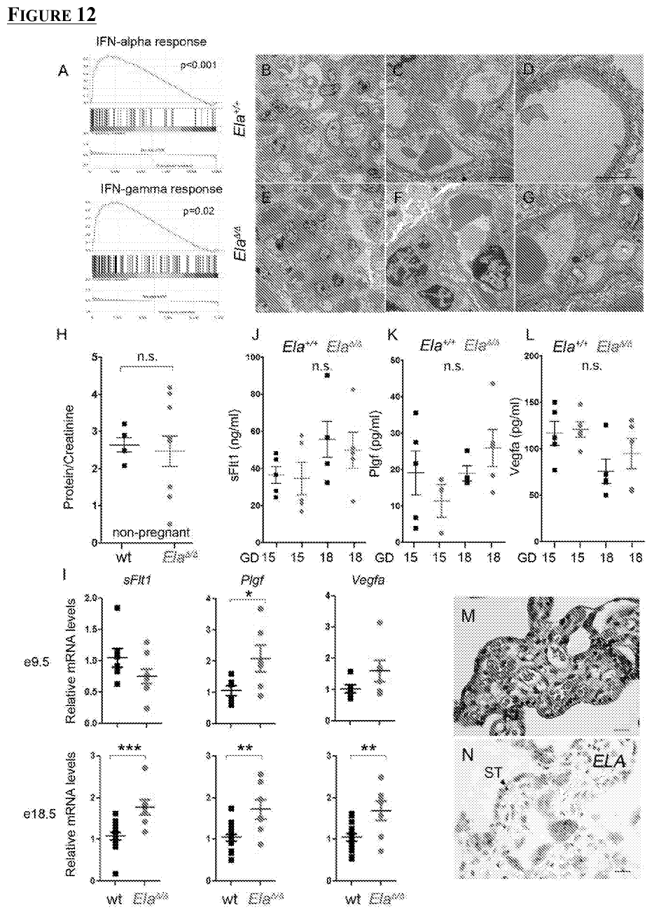

Defects in genes required for placental development and angiogenesis frequently lead to pre-eclampsia in mice (18, 19). In light of the placental defects seen in [symbol] conceptuses, including a prominent gene signature of increased inflammatory response (FIG. 12A), and increased expression of Esm1 (20, 21) and Adm (22), which have been linked to PE in humans, we hypothesized that Ela mothers might exhibit symptoms of PE.

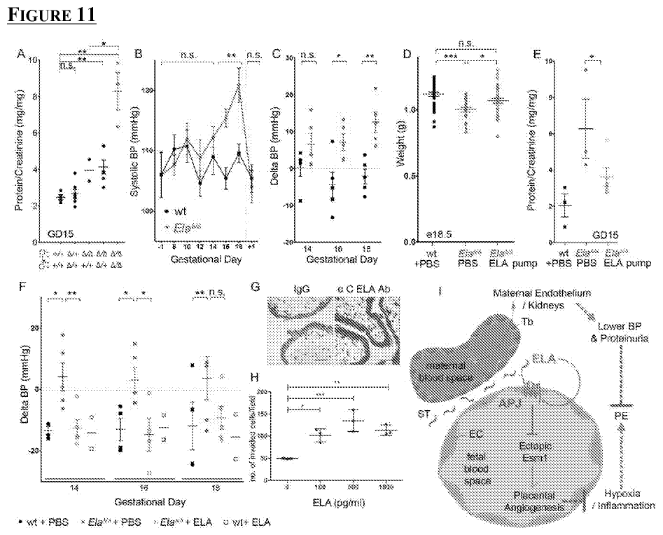

We thus assessed [symbol] pregnant mice for signs of proteinuria and hypertension, two diagnostic hallmarks of PE. Indeed, by decreasing the number of wt Ela alleles in fetuses and their pregnant mother, the urine protein/creatinine ratio at gestational day (GD) 15 increased dramatically, indicating an inverse correlation between endogenous ELA levels and the severity of proteinuria (FIG. 11A).

At the end of pregnancy, histology and transmission electron microscopy (TEM) of kidney glomerular sections revealed signs of endotheliosis in [symbol] pregnant mothers (FIG. 12B to FIG. 12G), a unique renal pathology of women suffering from PE (23, 24). Glomeruli from [symbol] pregnant mothers were swollen and had occluded capillaries, with evidence of protein and vesicular deposition on endothelial cells, absence of proper endothelial fenestration, and coagulation of red blood cells in capillary lumens (FIG. 12B to FIG. 12G). Podocytes on the other hand, appeared normal. Proteinuria was not observed in non-pregnant [symbol] mice (FIG. 12H), indicating that the renal pathology is unique to pregnancy.

Next, we employed a tail-cuff method to measure systolic blood pressure (BP) throughout pregnancy, after training the mice for a minimum of 3 days prior to mating. While there were no significant differences in the non-pregnant baseline BP between wt and [symbol] mice, pregnant [symbol] mothers (mated to [symbol] fathers) had significantly higher systolic BP, which returned to normal levels post-parturition (FIG. 11B), and delta BP (pregnant BP minus baseline BP) on GD 16 and 18 (FIG. 11C).

In addition, [symbol] pups from [symbol] mothers collected by caesarean section at e18.5 were significantly lower in weight compared to wt pups from wt mothers (FIG. 11D). This is reminiscent of intrauterine growth restriction (IUGR) that frequently accompanies PE and placental insufficiency. Our findings indicate that [symbol] mice suffer from PE, and suggest that ELA is necessary for regulating maternal cardiovascular homeostasis to prevent gestational hypertension.

To determine if the loss of ELA is upstream of well-established biomarkers of human PE, we measured both maternal plasma and placental mRNA levels of sFlt1 (24), Vegf (25) and Plgf (19, 26). At late gestation, [symbol] placentas have increased levels of sFlt1, Vegfa and Plgf mRNA (FIG. 12I), although these transcriptional changes did not translate into significantly elevated plasma levels of the respective proteins (FIG. 12J to FIG. 12L). Altogether, these data indicate that [symbol] mice are not developing PE symptoms simply due to a decrease in the Plgf/sFlt1 ratio but suggest that ELA acts independently of, and possibly earlier, than these angiogenic factors in the pathogenesis of PE.

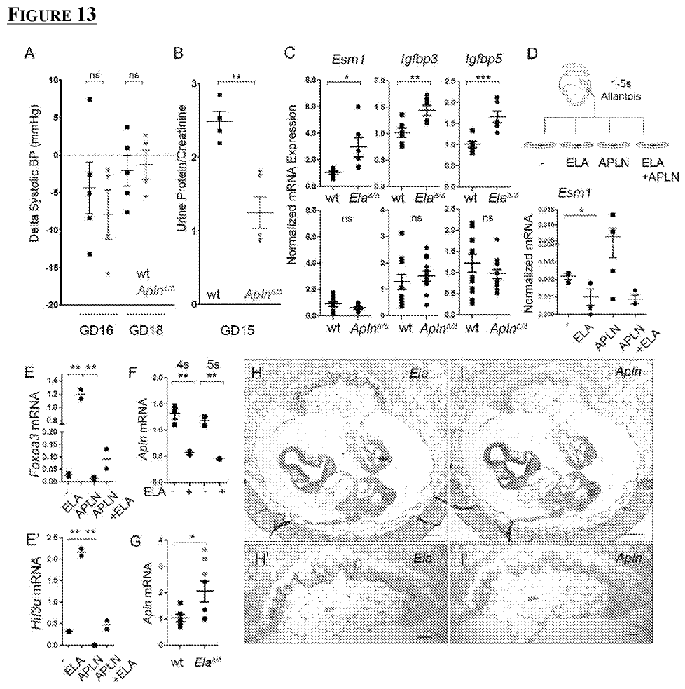

It is noteworthy that over-expression of Apln (FIG. 9F), which is the alternate ligand for APJ, is not sufficient to rescue [symbol] placentas, suggesting that these ligands elicit different signaling outcomes. Indeed, unlike [symbol] mothers, [symbol] mothers do not develop hypertension (FIG. 13A) and in fact have significantly lower levels of proteinuria (FIG. 13B).

Similarly, [symbol] placentas do not aberrantly upregulate endothelial tip cell markers Esm1 and Igfbp3 as seen in [symbol] placentas (FIG. 13C). To further investigate the biological differences between APLN and ELA, we treated APJ-expressing primary allantoic cultures from somite stage embryos with equal concentrations of ELA, APLN or both (FIG. 13D). We found that ELA and APLN elicited opposite effects on the expression of Esm1 and several hypoxic response genes (FIG. 13D to FIG. 13E′).

Lastly, we found that ELA could directly repress the expression of Apln in these allantoic explants (FIG. 13F), raising the possibility that Apln de-repression in the absence of ELA drives excessive and pathogenic tip cell differentiation. Indeed, we find that in a litter of [symbol] null embryos, the most severely-affected embryos are the ones expressing the highest levels of Apln (FIG. 13G). Moreover, the two ligands display distinct spatiotemporal expression where Ela is highly concentrated and restricted to the developing heart, caudal neural tube and trophoblasts whereas Apln is diffusely expressed and widespread in all embryonic and extraembryonic tissues (FIG. 13H to FIG. 13I′). Altogether, our data demonstrate that Ela and Apln are biologically distinct and elicit opposing effects on placental angiogenesis and symptoms of PE.

Since ELA appears to act as a systemic hormone during pregnancy, we asked whether administration of synthetic ELA during pregnancy may alleviate PE symptoms. ELA infusion did not affect BP and proteinuria parameters in pregnant wt mice (FIG. 11F) nor did it have noticeable side effects on embryogenesis, as measured by fetal weight, morphology and subsequent post-natal development. We were able to normalize proteinuria (FIG. 11E) and BP (FIG. 11F) in [symbol] pregnant mothers infused with recombinant ELA from GD 7.5 onwards, which is consistent with our model that ELA acts as a systemic hormone. Furthermore, infusion of ELA rescued the weight of [symbol] fetuses (FIG. 11D) and glomerular endotheliosis in pregnant [symbol] mothers as assayed by Periodic acid-Schiff and α-Fibrinogen staining (FIG. 14).

In humans, we find that ELA is predominantly expressed in villous cytotrophoblasts and syncytiotrophoblasts of first trimester placental tissue (8+3 weeks) (FIG. 11G) and term placentas (FIG. 12M and FIG. 12N). In PE, extravillous trophoblast invasion and subsequent spiral artery remodeling are frequently incomplete, leading to shallow and defective placentation (27).

We therefore hypothesized that in humans, ELA might contribute to trophoblast invasion. Indeed, addition of ELA to trophoblast-like JAR choriocarcinoma cells significantly increased their invasiveness in transwell invasion assays (FIG. 11H) (28).

These data suggest that ELA, secreted from the syncytiotrophoblast layer has a paracrine effect on trophoblasts differentiating into invasive extravillous trophoblasts. ELA activity potentiates invasion and might enhance subsequent spiral artery remodeling to prevent the development of PE during human pregnancy.

### Example 26. Discussion

Pregnancy is a unique physiological state associated with increased cardiovascular demand and burden. Many processes work in concert to impart cardiovascular homeostasis in the pregnant female, although to date these are largely unknown. In the mouse, we propose that ELA produced by placental trophoblasts functions in two ways to prevent gestational hypertension (FIG. 11I).

First, ELA exerts paracrine effects on fetal endothelial cells, where it curbs inappropriate differentiation of endothelial tips cells. This enables normal branching angiogenesis and the formation of an adequate labyrinth network required for proper perfusion.

Secondly, ELA enters the maternal circulation to regulate cardio-renal function. We speculate that the latter role might have a direct effect on the maternal endothelium (e.g. through the stimulation of vasodilatory mechanisms such as nitric oxide production) (29), or by regulating diuresis (30).

While the PE-protective effects are presumably achieved through APJ signaling in endothelial cells, we do not rule out a possible contribution from as yet identified ELA receptors (1). ELA deficiency in the mouse leads to classical PE symptoms together with gross abnormalities in placental development.

Similarly, ELA is expressed by trophoblasts in the chorionic villi of human placentas, and potentiates trophoblast invasion in vitro. We speculate that in humans ELA might contribute to placentation by stimulating trophoblast migration and invasion, in addition to direct effects on the maternal endothelium, although these remain to be investigated.

In conclusion, we propose that ELA is a circulating hormone produced by the mammalian placenta to ensure cardiovascular integrity of both mother and fetus during pregnancy. Our results raise the possibility that transient enforcement of the ELABELA-nergic axis might therefore be beneficial for conditions that display hypertension such as, but not limited to, pre-eclampsia.

### REFERENCES

ELABELA: a hormone essential for heart development signals via the apelin receptor. Chng SC*, Ho L*, Tian J, Reversade B. Dev Cell. 2013 Dec. 23; 27(6):672-80.
ELABELA Is an Endogenous Growth Factor that Sustains hESC Self-Renewal via the PI3K/AKT Pathway. Ho L, Tan S Y, Wee S, Wu Y, Tan S J, Ramakrishna N B, Chng S C, Nama S, Szczerbinska I, Chan Y S, Avery S, Tsuneyoshi N, Ng H H, Gunaratne J, Dunn N R, Reversade B. Cell Stem Cell. 2015 Oct. 1; 17(4):435-47.
Discovery and Structure-Activity Relationship of a Bioactive Fragment of ELABELA that Modulates Vascular and Cardiac Functions. Murza A, Sainsily X, Coquerel D, Côté J, Marx P, Besserer-Offroy É, Longpré J M, Lainé J, Reversade B, Salvail D, Leduc R, Dumaine R, Lesur O, Auger-Messier M, Sarret P, Marsault É. J Med Chem. 2016 Apr. 14; 59(7):2962-72.
Characterization of apela, a novel endogenous ligand of apelin receptor, in the adult heart. Perjés Á, Kilpiö T, Ulvila J, Magga J, Alakoski T, Szabó Z, Vainio L, Halmetoj a E, Vuolteenaho O, Petäjä-Repo U, Szokodi I, Kerkelä R. Basic Res Cardiol. 2016 January; 111(1):2.
The hormonal peptide Elabela guides angioblasts to the midline during vasculogenesis. Helker C S, Schuermann A, Pollmann C, Chng S C, Kiefer F, Reversade B, Herzog W. Elife. 2015 May 27; 4.
1. L. Ho et al., ELABELA Is an Endogenous Growth Factor that Sustains hESC Self-Renewal via the PI3K/AKT Pathway. Cell Stem Cell 17, 435-447 (2015).
2. S. C. Chng, L. Ho, J. Tian, B. Reversade, ELABELA: a hormone essential for heart development signals via the apelin receptor. Dev Cell 27, 672-680 (2013).
3. A. Pauli et al., Toddler: an embryonic signal that promotes cell movement via Apelin receptors. Science 343, 1248636 (2014).
4. C. S. Helker et al., The hormonal peptide Elabela guides angioblasts to the midline during vasculogenesis. Elife 4, (2015).
5. M. Li et al., An Apela RNA-Containing Negative Feedback Loop Regulates p53-Mediated Apoptosis in Embryonic Stem Cells. Cell Stem Cell 16, 669-683 (2015).
6. A. S. Hassan, J. Hou, W. Wei, P. A. Hoodless, Expression of two novel transcripts in the mouse definitive endoderm. Gene Expr Patterns 10, 127-134 (2010).
7. A. Subramanian et al., Gene set enrichment analysis: a knowledge-based approach for interpreting genome-wide expression profiles. Proc Natl Acad Sci USA 102, 15545-15550 (2005).
8. T. Hagen, C. T. Taylor, F. Lam, S. Moncada, Redistribution of intracellular oxygen in hypoxia by nitric oxide: effect on HIF1 alpha. Science 302, 1975-1978 (2003).
9. R. del Toro et al., Identification and functional analysis of endothelial tip cell-enriched genes. Blood 116, 4025-4033 (2010).
10. H. M. Eilken, R. H. Adams, Dynamics of endothelial cell behavior in sprouting angiogenesis. Curr Opin Cell Biol 22, 617-625 (2010).
11. P. Carmeliet, Angiogenesis in health and disease. Nat Med 9, 653-660 (2003).
12. R. H. Adams, A. Eichmann, Axon guidance molecules in vascular patterning. Cold Spring Harb Perspect Biol 2, a001875 (2010).
13. S. F. Rocha et al., Esm1 modulates endothelial tip cell behavior and vascular permeability by enhancing VEGF bioavailability. Circ Res 115, 581-590 (2014).
14. G. A. Strasser, J. S. Kaminker, M. Tessier-Lavigne, Microarray analysis of retinal endothelial tip cells identifies CXCR4 as a mediator of tip cell morphology and branching. Blood 115, 5102-5110 (2010).
15. D. N. Charo et al., Endogenous regulation of cardiovascular function by apelin-APJ. Am J Physiol Heart Circ Physiol 297, H1904-1913 (2009).
16. S. Suchting et al., The Notch ligand Delta-like 4 negatively regulates endothelial tip cell formation and vessel branching. Proc Natl Acad Sci USA 104, 3225-3230 (2007).
17. A. Duarte et al., Dosage-sensitive requirement for mouse Dll4 in artery development. Genes Dev 18, 2474-2478 (2004).
18. J. Singh, A. Ahmed, G. Girardi, Role of complement component C1q in the onset of preeclampsia in mice. Hypertension 58, 716-724 (2011).
19. S. Venkatesha et al., Soluble endoglin contributes to the pathogenesis of preeclampsia. Nat Med 12, 642-649 (2006).
20. X. Chang et al., Endocan of the maternal placenta tissue is increased in pre-eclampsia. Int J Clin Exp Pathol 8, 14733-14740 (2015).
21. H. Adekola et al., Endocan, a putative endothelial cell marker, is elevated in preeclampsia, decreased in acute pyelonephritis, and unchanged in other obstetrical syndromes. J Matern Fetal Neonatal Med 28, 1621-1632 (2015).
22. A. Al-Ghafra, N. M. Gude, S. P. Brennecke, R. G. King, Increased adrenomedullin protein content and mRNA expression in human fetal membranes but not placental tissue in pre-eclampsia. Mol Hum Reprod 12, 181-186 (2006).
23. I. E. Stillman, S. A. Karumanchi, The glomerular injury of preeclampsia. J Am Soc Nephrol 18, 2281-2284 (2007).
24. S. E. Maynard et al., Excess placental soluble fms-like tyrosine kinase 1 (sFlt1) may contribute to endothelial dysfunction, hypertension, and proteinuria in preeclampsia. J Clin Invest 111, 649-658 (2003).
25. R. Hayman, J. Brockelsby, L. Kenny, P. Baker, Preeclampsia: the endothelium, circulating factor(s) and vascular endothelial growth factor. J Soc Gynecol Investig 6, 3-10 (1999).
26. A. Reuvekamp, F. V. Velsing-Aarts, I. E. Poulina, J. J. Capello, A. J. Duits, Selective deficit of angiogenic growth factors characterises pregnancies complicated by pre-eclampsia. Br J Obstet Gynaecol 106, 1019-1022 (1999).
27. S. J. Fisher, Why is placentation abnormal in preeclampsia? Am J Obstet Gynecol 213, S115-122 (2015).
28. S. Yagel, R. S. Parhar, J. J. Jeffrey, P. K. Lala, Normal nonmetastatic human trophoblast cells share in vitro invasive properties of malignant cells. J Cell Physiol 136, 455-462 (1988).
29. C. A. Schreiber, S. J. Holditch, A. Generous, Y. Ikeda, Sustained ELABELA Gene Therapy in High Salt-Induced Hypertensive Rats. Curr Gene Ther, (2016).
30. C. Deng, H. Chen, N. Yang, Y. Feng, A. J. Hsueh, Apela Regulates Fluid Homeostasis by Binding to the APJ Receptor to Activate Gi Signaling. J Biol Chem 290, 18261-18268 (2015).
31. N. Barker, H. Clevers, Lineage tracing in the intestinal epithelium. Curr Protoc Stem Cell Biol Chapter 5, Unit5A 4 (2010).
32. T. Yokomizo et al., Whole-mount three-dimensional imaging of internally localized immunostained cells within mouse embryos. Nat Protoc 7, 421-431 (2012).
33. Y. Liu, M. Antonyak, X. Peng, Visualization of mouse embryo angiogenesis by fluorescence-based staining. Methods Mol Biol 843, 79-85 (2012).
34. S. Devi et al., Platelet recruitment to the inflamed glomerulus occurs via an alphaIIbbeta3/GPVI-dependent pathway. Am J Pathol 177, 1131-1142 (2010).
35. J. Schindelin et al., Fiji: an open-source platform for biological-image analysis. Nat Methods 9, 676-682 (2012).
36. S. Bolte, F. P. Cordelieres, A guided tour into subcellular colocalization analysis in light microscopy. J Microsc 224, 213-232 (2006).
A1. L. Ho et al., ELABELA Is an Endogenous Growth Factor that Sustains hESC Self-Renewal via the PI3K/AKT Pathway. Cell Stem Cell 17, 435-447 (2015).
A2. S. C. Chng, L. Ho, J. Tian, B. Reversade, ELABELA: a hormone essential for heart development signals via the apelin receptor. Dev Cell 27, 672-680 (2013).
A3. A. Pauli et al., Toddler: an embryonic signal that promotes cell movement via Apelin receptors. Science 343, 1248636 (2014).
A4. C. S. Helker et al., The hormonal peptide Elabela guides angioblasts to the midline during vasculogenesis. Elife 4, (2015).
A5. M. Li et al., An Apela RNA-Containing Negative Feedback Loop Regulates p53-Mediated Apoptosis in Embryonic Stem Cells. Cell Stem Cell 16, 669-683 (2015).
A6. A. S. Hassan, J. Hou, W. Wei, P. A. Hoodless, Expression of two novel transcripts in the mouse definitive endoderm. Gene Expr Patterns 10, 127-134 (2010).
A7. A. Subramanian et al., Gene set enrichment analysis: a knowledge-based approach for interpreting genome-wide expression profiles. Proc Natl Acad Sci USA 102, 15545-15550 (2005).
A8. T. Hagen, C. T. Taylor, F. Lam, S. Moncada, Redistribution of intracellular oxygen in hypoxia by nitric oxide: effect on HIF1 alpha. Science 302, 1975-1978 (2003).
A9. R. del Toro et al., Identification and functional analysis of endothelial tip cell-enriched genes. Blood 116, 4025-4033 (2010).
A10. H. M. Eilken, R. H. Adams, Dynamics of endothelial cell behavior in sprouting angiogenesis. Curr Opin Cell Biol 22, 617-625 (2010).
A11. P. Carmeliet, Angiogenesis in health and disease. Nat Med 9, 653-660 (2003).
A12. R. H. Adams, A. Eichmann, Axon guidance molecules in vascular patterning. Cold Spring Harb Perspect Biol 2, a001875 (2010).
A13. S. F. Rocha et al., Esm1 modulates endothelial tip cell behavior and vascular permeability by enhancing VEGF bioavailability. Circ Res 115, 581-590 (2014).
A14. G. A. Strasser, J. S. Kaminker, M. Tessier-Lavigne, Microarray analysis of retinal endothelial tip cells identifies CXCR4 as a mediator of tip cell morphology and branching. Blood 115, 5102-5110 (2010).
A15. D. N. Charo et al., Endogenous regulation of cardiovascular function by apelin-APJ. Am J Physiol Heart Circ Physiol 297, H1904-1913 (2009).
A16. S. Suchting et al., The Notch ligand Delta-like 4 negatively regulates endothelial tip cell formation and vessel branching. Proc Natl Acad Sci USA 104, 3225-3230 (2007).
A17. A. Duarte et al., Dosage-sensitive requirement for mouse Dll4 in artery development. Genes Dev 18, 2474-2478 (2004).
A18. J. Singh, A. Ahmed, G. Girardi, Role of complement component C1q in the onset of preeclampsia in mice. Hypertension 58, 716-724 (2011).
A19. S. Venkatesha et al., Soluble endoglin contributes to the pathogenesis of preeclampsia. Nat Med 12, 642-649 (2006).
A20. X. Chang et al., Endocan of the maternal placenta tissue is increased in pre-eclampsia. Int J Clin Exp Pathol 8, 14733-14740 (2015).
A21. H. Adekola et al., Endocan, a putative endothelial cell marker, is elevated in preeclampsia, decreased in acute pyelonephritis, and unchanged in other obstetrical syndromes. J Matern Fetal Neonatal Med 28, 1621-1632 (2015).
A22. A. Al-Ghafra, N. M. Gude, S. P. Brennecke, R. G. King, Increased adrenomedullin protein content and mRNA expression in human fetal membranes but not placental tissue in pre-eclampsia. Mol Hum Reprod 12, 181-186 (2006).
A23. I. E. Stillman, S. A. Karumanchi, The glomerular injury of preeclampsia. J Am Soc Nephrol 18, 2281-2284 (2007).
A24. S. E. Maynard et al., Excess placental soluble fms-like tyrosine kinase 1 (sFlt1) may contribute to endothelial dysfunction, hypertension, and proteinuria in preeclampsia. J Clin Invest 111, 649-658 (2003).
A25. R. Hayman, J. Brockelsby, L. Kenny, P. Baker, Preeclampsia: the endothelium, circulating factor(s) and vascular endothelial growth factor. J Soc Gynecol Investig 6, 3-10 (1999).
A26. A. Reuvekamp, F. V. Velsing-Aarts, I. E. Poulina, J. J. Capello, A. J. Duits, Selective deficit of angiogenic growth factors characterises pregnancies complicated by pre-eclampsia. Br J Obstet Gynaecol 106, 1019-1022 (1999).
A27. S. J. Fisher, Why is placentation abnormal in preeclampsia? Am J Obstet Gynecol 213, S115-122 (2015).
A28. S. Yagel, R. S. Parhar, J. J. Jeffrey, P. K. Lala, Normal nonmetastatic human trophoblast cells share in vitro invasive properties of malignant cells. J Cell Physiol 136, 455-462 (1988).
A29. C. A. Schreiber, S. J. Holditch, A. Generous, Y. Ikeda, Sustained ELABELA Gene Therapy in High Salt-Induced Hypertensive Rats. Curr Gene Ther, (2016).
A30. C. Deng, H. Chen, N. Yang, Y. Feng, A. J. Hsueh, Apela Regulates Fluid Homeostasis by Binding to the APJ Receptor to Activate Gi Signaling. J Biol Chem 290, 18261-18268 (2015).
A31. N. Barker, H. Clevers, Lineage tracing in the intestinal epithelium. Curr Protoc Stem Cell Biol Chapter 5, Unit5A 4 (2010).
A32. T. Yokomizo et al., Whole-mount three-dimensional imaging of internally localized immunostained cells within mouse embryos. Nat Protoc 7, 421-431 (2012).
A33. Y. Liu, M. Antonyak, X. Peng, Visualization of mouse embryo angiogenesis by fluorescence-based staining. Methods Mol Biol 843, 79-85 (2012).
A34. S. Devi et al., Platelet recruitment to the inflamed glomerulus occurs via an alphaIIbbeta3/GPVI-dependent pathway. Am J Pathol 177, 1131-1142 (2010).
A35. J. Schindelin et al., Fiji: an open-source platform for biological-image analysis. Nat Methods 9, 676-682 (2012).
A36. S. Bolte, F. P. Cordelieres, A guided tour into subcellular colocalization analysis in light microscopy. J Microsc 224, 213-232 (2006).

In this document and in its claims, the verb “to comprise” and its conjugations is used in its non-limiting sense to mean that items following the word are included, but items not specifically mentioned are not excluded. In addition, reference to an element by the indefinite article “a” or “an” does not exclude the possibility that more than one of the element is present, unless the context clearly requires that there be one and only one of the elements. The indefinite article “a” or “an” thus usually means “at least one”.

Each of the applications and patents mentioned in this document, and each document cited or referenced in each of the above applications and patents, including during the prosecution of each of the applications and patents (“application cited documents”) and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the claims.

1. (canceled)

2. (canceled)

3. (canceled)

4. (canceled)

5. (canceled)

6. (canceled)

7. (canceled)

8. (canceled)

9. (canceled)

10. (canceled)

11. (canceled)

12. (canceled)

13. (canceled)

14. (canceled)

15. (canceled)

16. (canceled)

17. (canceled)

18. (canceled)

19. (canceled)

20. (canceled)

21. A method of treatment, alleviation or prophylaxis of pre-eclampsia in an individual, the method comprising administering a therapeutically effective amount of an ELABELA polypeptide or a nucleic acid encoding an ELABELA polypeptide to the individual.

22. The method of claim 21, in which the ELABELA polypeptide comprises a sequence CXXXRCXXXHSRVPFP (SEQ ID NO: 1), in which X represents an amino acid residue.

23. The method of claim 21, in which the ELABELA polypeptide is selected from one or more of the following:
(a) a sequence SEQ ID NO: 162 (XXXXXXXCXXXRCXXXHSRVPFP), in which X represents an amino acid residue, and in which position 1 of SEQ ID NO: 162 comprises a basic amino acid residue;
(b) a sequence SEQ ID NO: 163 (XXXXXXXXCXXXRCXXXHSRVPFP), in which X represents an amino acid residue, and in which position 1 of SEQ ID NO: 163 comprises a basic amino acid residue; and
(c) a sequence SEQ ID NO: 163 (XXXXXXXXCXXXRCXXXHSRVPFP), in which X represents an amino acid residue, in which positions 1 and 2 of SEQ ID NO: 163 comprise a pair of basic amino acid residues.

24. The method of claim 23, in which the ELABELA polypeptide comprises one or more of the following:
(a) a sequence SEQ ID NO: 162 (XXXXXXXCXXXRCXXXHSRVPFP), in which X represents an amino acid residue, and in which position 1 of SEQ ID NO: 162 comprises K or R;
(b) a sequence SEQ ID NO: 163 (XXXXXXXXCXXXRCXXXHSRVPFP), in which X represents an amino acid residue, and in which position 1 of SEQ ID NO: 163 comprises K or R;
(c) a sequence SEQ ID NO: 163 (XXXXXXXXCXXXRCXXXHSRVPFP), in which X represents an amino acid residue, in which positions 1 and 2 of SEQ ID NO: 163 comprise a pair of basic amino acid residues selected from KK, KR, RK or RR.

25. The method of claim 21, in which the ELABELA polypeptide comprises a sequence selected from the group consisting of: SEQ ID NO: 2 to SEQ ID NO: 18.

26. The method of claim 25, in which the ELABELA polypeptide comprises a sequence of human ELABELA of SEQ ID NO: 2.

27. The method of claim 21, in which the ELABELA polypeptide further comprises a human ELABELA signal sequence of SEQ ID NO: 19.

28. The method of claim 21, in which the ELABELA polypeptide comprises a sequence selected from the group consisting of: SEQ ID NO: 20 to SEQ ID NO: 36.

29. The method of claim 28, in which the ELABELA polypeptide comprises a human ELABELA sequence of SEQ ID NO: 20.

30. The method of claim 21, in which the ELABELA polypeptide comprises an intramolecular covalent bond between cysteine residues at or about positions 1 and 6, with reference to the numbering in the sequence CXXXRCXXXHSRVPFP (SEQ ID NO: 1), or in which one or both cysteine residues comprise a reduced cysteine having a sulfhydryl group.

31. The method of claim 21, in which the ELABELA polypeptide comprises any one or more of the following:
(a) a mutation of a basic residue at position 31, with reference to the position numbering of a human ELABELA of SEQ ID NO: 20;
(b) a mutation of a basic residue at position 32, with reference to the position numbering of a human ELABELA of SEQ ID NO: 20;
(c) a R31G, R31 A, K31G or K31 A substitution, with reference to the position numbering of a human ELABELA of SEQ ID NO: 20.

32. The method of claim 31, wherein the ELABELA polypeptide comprises one or more of the following with reference to the position numbering of a human ELABELA polypeptide of SEQ ID NO: 20;
(a) a mutation of a basic residue at position 31, in which the basic residue at position 31 is mutated to a neutral residue, or in which K or R at position 31 is mutated to A or G;
(b) a mutation of a basic residue at position 32, in which the basic residue at position 32 is mutated to a neutral residue, or in which K or R at position 32 is mutated to A or G; or
(c) a R31G, R31A, K31G, K31A, R32G, R32A, K32G or K32A substitution.

33. The method of claim 21, wherein the nucleic acid encoding an ELABELA polypeptide comprises a nucleic acid sequence in any of SEQ ID NO. 37 to SEQ ID NO: 46.

34. The method of claim 33, wherein the nucleic acid encoding an ELABELA polypeptide comprises a nucleic acid sequence of SEQ ID NO: 37 or SEQ ID NO: 42.

35. The method of claim 21, wherein the nucleic acid is comprised by a vector.

36. A method of assaying a compound useful in the treatment or alleviation of preeclampsia, the method comprising:
(a) contacting an ELABELA polypeptide with a candidate compound and performing an assay to determine if the candidate compound binds to the ELABELA polypeptide;
(b) contacting an ELABELA polypeptide with a candidate compound and performing an assay to determine if the candidate compound modulates an activity of the ELABELA polypeptide; or
(c) contacting a cell expressing an ELABELA polypeptide with a candidate compound and performing an assay to determine if the candidate compound causes an elevated or reduced expression, amount or activity of the ELABELA polypeptide in or of the cell, wherein a candidate compound that causes elevated or reduced expression, amount or activity of the ELABELA polypeptide identifies that compound as a candidate compound useful in the treatment or alleviation of preeclampsia.

37. The method of claim 36, further comprising isolating or synthesizing the compound of interest so identified.
